(19) United States
(12) Patent Application Publication
(10) Pub. No.: US 2025/0230125 A1
(43) Pub. Date: Jul. 17, 2025

(54) Small Molecule Allosteric Modulators of Serotonin (5-HT) 5-HT2C and 5-HT2A Receptors

(71) Applicant: The Board of Regents of The University of Texas System, Austin, TX (US)

(72) Inventors: Jia Zhou, Galveston, TX (US); Jianping Chen, Galveston, TX (US); Christopher T. Wild, Galveston, TX (US); Noelle C. Anastasio, Galveston, TX (US); Kathryn A. Cunningham, Galveston, TX (US)

(21) Appl. No.: 18/853,376

(22) PCT Filed: Feb. 1, 2023
(86) PCT No.: PCT/US2023/012161

§ 371 Date: Oct. 1, 2024

Related U.S. Application Data
(60) Provisional application No. 63/326,600, filed on Apr. 1, 2022.

(51) Int. Cl. C07C 233/20; A61K 31/16; A61K 31/165; A61K 31/19; A61K 31/336; A61K 31/396; A61K 31/4045; A61K 31/405; A61K 31/5375; C07C 233/19; C07C 233/22; C07C 233/27; C07C 233/29; C07C 233/48; C07C 233/49; C07D 203/08; C07D 209/18; C07D 295/145; C07D 303/46; C07D 403/12

(52) U.S. Cl. CPC C07C 233/20; A61K 31/16; A61K 31/165; A61K 31/19; A61K 31/336; A61K 31/396; A61K 31/4045; A61K 31/405; A61K 31/5375; C07C 233/19; C07C 233/22; C07C 233/27; C07C 233/29; C07C 233/48; C07C 233/49; C07D 203/08; C07D 209/18; C07D 295/145; C07D 303/46; C07D 403/12; C07C 2601/02

(57) ABSTRACT

The present invention relates to novel 5HT receptor modulators, such as compounds of the general Formula (I) and general Formula (II):

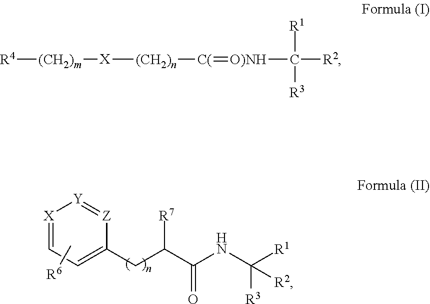

or a pharmaceutically acceptable salt thereof. Methods of using the compounds include, for example, modulation of 5-hydroxytryptamine (5-HT) receptor subtypes, including 5-hydroxytryptamine 2A receptor and/or 5-hydroxytryptamine 2C receptor, and treatment of a condition or disease where the treatment is associated with the modulation of 5-hydroxytryptamine 2A receptor and/or 5-hydroxytryptamine 2C receptor.

### CROSS REFERENCE TO RELATED APPLICATIONS

This application claims priority to U.S. Application No. 63/326,600, filed on Apr. 1, 2022, the contents of which is hereby incorporated by reference in its entirety.

### STATEMENT OF FEDERALLY SPONSORED RESEARCH AND DEVELOPMENT

This invention was made with government support under the Grants R21 MH093844, R01 DA038446, T32 DA007287, and F31 DA038922 awarded by the National Institutes of Health (NIH). The government has certain rights in the invention.

### FIELD OF THE INVENTION

The field of the invention relates generally to novel small molecules that bind and/or modulate serotonin receptor subtypes as well as the preparation and the use thereof.

### BRIEF DESCRIPTION OF THE FIGURES

FIGS. 1A-1C show the chemical structures of certain embodiments of olefinic compounds according to the invention.

FIG. 2 shows the chemical structures of certain embodiments of epoxy compounds according to the invention.

FIG. 3 shows the chemical structures of certain embodiments of aziridine and cyclopropane compounds according to the invention.

FIG. 4 shows the chemical structures of certain embodiments of aliphatic amide compounds according to the invention.

FIG. 5 shows the chemical structures of certain embodiments of arylalkylamide compounds according to the invention.

FIG. 6 shows the chemical structures of polar head (“PH”) substituents included in certain embodiments of arylalkylamide compounds according to the invention.

FIG. 7 sections A-Y are graphs of concentration-response curves of compounds 6-30 according to the present invention (1 nM) on Caᵢ²⁺ release induced by 5-HT in live h5-HT_{2C}R-CHO cells.

FIG. 8 sections A-J are graphs of concentration-response curves of select compounds according to the present invention (1 nM) on Caᵢ²⁺ release induced by 5-HT in live h5-HT_{2A}R-CHO cells.

FIG. 9 sections A-F are graphs of concentration-response curves of select compounds according to the present invention (1 nM) on Caᵢ²⁺ release induced by 5-HT in live h5-HT_{2B}R-CHO cells.

FIGS. 10A and 10B are graphs of concentration-response curves of select compounds according to the present invention (1 nM) on Caᵢ²⁺ release induced by 5-HT in (FIG. 10A) live h5-HT_{2C}R-CHO cells and in (FIG. 10B) h5-HT_{2A}R-CHO cells, respectively.

FIGS. 11A and 11B are graphs of concentration-response curves of select compounds according to the present invention (1 nM) on Caᵢ²⁺ release induced by 5-HT in (FIG. 11A) live h5-HT_{2C}R-CHO cells and in (FIG. 11B) h5-HT_{2A}R-CHO cells, respectively.

FIGS. 12A and 12B are ¹H and ¹³C NMR spectra, respectively, of compound 13 (JPC0323) according to the present invention.

### DESCRIPTION

All publications mentioned herein are incorporated by reference to the extent they support the present invention.

### 1.0 Definitions

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated invention, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The use of “or” means “and/or” unless stated otherwise.

The use of “a” herein means “one or more” unless stated otherwise or where the use of “one or more” is clearly inappropriate.

The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to a ±10% variation from the nominal value. It is to be understood that such a variation is always included in any given value provided herein, whether or not it is specifically referred to.

The term “pharmaceutically acceptable salt” refers to those salts of the compounds of the present invention which are, within the scope of sound medical judgment, suitable for use in contact with the tissues of patients without undue toxicity, and the like. As used herein, the term “pharmaceutically acceptable salt” may include acetate, hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. (See S. M. Barge et al., “Pharmaceutical Salts,” J. Pharm. Sci., 66:1-19 (1977), which is incorporated herein by reference in its entirety, for further examples of pharmaceutically acceptable salt).

The term “HBTU” refers to 3-[Bis(dimethylamino)methyliumyl]-3H-benzotriazol-1-oxide hexafluorophosphate (also known as 2-(1H-benzotriazol-1-yl)-1,1,3,3-tetramethyluronium hexafluorophosphate).

The term “HOBt” refers the following structure, known as 1-hydroxybenzotriazole, (including hydrates and polymorphs, thereof):

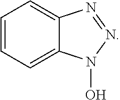

The term “DIEA” refers to N,N-Diisopropylethylamine (also known as Hünig's base, DIPEA, and ethyldiisopropylamine).

The term “DCM” refers to dichloromethane (also known as methylene chloride).

The term “TFA” refers to trifluoroacetic acid.

The term “rt” refers to room temperature.

The term “alkyl” as used herein by itself or as part of another group refers to both straight and branched chain radicals, and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The term “alkyl” may include methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, undecyl, and dodecyl.

The term “heteroalkyl” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of O, and N. The heteroatom(s) may be placed at any interior position of the heteroalkyl group or at the position at which the alkyl group is attached to the remainder of the molecule. Up to two heteroatoms may be consecutive.

The term “alkylene” as used herein refers to straight and branched chain alkyl linking groups, i.e., an alkyl group that links one group to another group in a molecule. In some embodiments, the term “alkylene” may include —(CH₂)ₙ— where n is 2-8.

The term “aryl” means a polyunsaturated hydrocarbon substituent. Aryl groups can be monocyclic or polycyclic (e.g., 2 to 3 rings that are fused together or linked covalently). Non-limiting examples of aryl and heteroaryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

The term “heteroaryl” as used herein refers to groups having 5 to 14 ring atoms; 6, 10 or 14 7π-electrons shared in a cyclic array; and containing carbon atoms and 1, 2 or 3 oxygen, nitrogen or sulfur heteroatoms. A heteroaryl group can be attached to the remainder of the molecule through a carbon or heteroatom. Especially preferred heteroaryl groups include 1,2,3-triazole, 1,2,4-triazole, 5-amino 1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, 2-aminopyridine, 4-aminopyridine, 2-aminoimidazoline, and 4-aminoimidazoline.

An “amino” group refers to an —NH₂ group.

An “amido” group refers to an —CONH₂ group. An alkylamido group refers to an —CONHR group wherein R is as defined above. A dialkylamido group refers to an —CONRR′ group wherein R and R′ are as defined above.

The term “halogen” or “halo” as used herein by itself or as part of another group refers to chlorine, bromine, fluorine or iodine.

The term “hydroxy” or “hydroxyl” as used herein by itself or as part of another group refers to an —OH group.

An “alkoxy” group refers to an —O-alkyl group wherein “alkyl” is as defined above. In one embodiment, the alkyl group has 1-12 carbons. In another embodiment, the alkyl group has 1-7 carbons. In a further embodiment, the alkyl group has 1-6 carbons. In another embodiment, the alkyl group has 1-4 carbons.

A “thio” group refers to an —SH group.

An “alkylthio” group refers to an —SR group wherein R is alkyl as defined above.

The term “heterocycle” or “heterocyclic ring”, as used herein except where noted, represents a stable 5- to 7-membered monocyclic-, or stable 7- to 11-membered bicyclic heterocyclic ring system, any ring of which may be saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. Rings may contain one oxygen or sulfur, one to three nitrogen atoms, or one oxygen or sulfur combined with one or two nitrogen atoms. The heterocyclic ring may be attached at any heteroatom or carbon atom that results in the creation of a stable structure.

The term “alkylamino” as used herein by itself or as part of another group refers to an amino group which is substituted with one alkyl group having from 1 to 6 carbon atoms. The term “dialkylamino” as used herein by itself or as part of another group refers to an amino group which is substituted with two alkyl groups, each having from 1 to 6 carbon atoms.

The term “arylamine” or “arylamino” as used herein by itself or as part of another group refers to an amino group which is substituted with an aryl group, as defined above.

As used herein, the term “arylalkyl” denotes an alkyl group substituted with an aryl group, for example, Ph-CH₂— etc.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, alkyl, heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl (—C(O)NR₂), unsubstituted alkyl, unsubstituted heteroalkyl, alkoxy, alkylthio, alkylamino, (alkyl)₂amino, alkylsulfinyl, alkyl sulfonyl, aryl sulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

As used herein, the term “hydroxyalkyl” refers to an alkyl group (as defined above) substituted with a hydroxy substituent. In certain aspects, a hydroxyalkyl group may optionally be further substituted with additional hydroxy substitutents, to provide, for example, dihydroxyalkyl and trihydroxyalkyl groups, including C1 to C6 dihydroxyalkyl group and C1 to C6 trihydroxyalkyl groups. Exemplary hydroxyalkyl groups with additional hydroxy substitutents can include, but are not limited to, —CH₂CH(OH)CH₂OH and —CH₂CH(OH)CH(OH)CH₃.

### 2.0 Abbreviations

Serotonin, 5-HT; 5-HT_{2A} receptor, 5-HT_{2A}R; 5-HT_{2B} receptor, 5-HT_{2B}R; 5-HT_{2C} receptor, 5-HT_{2C}R; serotonin 5-HT₂ receptors, 5-HT₂Rs; positive allosteric modulators, PAMs; central nervous system, CNS; G protein-coupled receptors, GPCRs; cocaine use disorder, CUD; d-lysergic acid diethylamide, LSD; major depressive disorder, MDD; lipophilic tail, LT; polar head, PH; extracellular loop 2, ECL2; structure-activity relationship, SAR; N,N,N′,N′-tetramethyl-O-(1H-benzotriazol-1-yl)uronium hexafluorophosphate, HBTU; 1-ethyl-(3-dimethylaminopropyl)carbodiimide hydrochloride, EDCI; N,N-diisopropylethylamine, DIPEA; trifluoroacetic acid, TFA; preparative thin layer chromatographic, PTLC; phospholipase Cβ, PLCβ; intracellular calcium, Ca²⁺; Chinese hamster ovary, CHO; maximum 5-HT-induced Caᵢ²⁺ release, Eₘₐₓ; negative allosteric modulator, NAM; national institute of mental health, NIMH; psychoactive drug screening program, PDSP; multiparameter optimization, MPO; P-glycoprotein, P-gp; pharmacokinetics, PK; induced Fit Docking, IFD; extra-precision, XP; standard-precision, SP; extracellular loop, ECL; transmembrane helix, TM; human ether-a-go-go-related gene, hERG; half-life, T_{1/2}.

### 3.0 Compound Identifiers

Compound identifiers are included herein in some locations with numeric identifiers (e.g., 1, 2, 3 . . . 30), and in some other locations in the description the same structures are referred to with alphanumeric identifiers. The compound identifiers are used interchangeably herein, according to the following list of equivalent compound identifiers:

| Numeric | Alphanumeric |
|---|---|
| Compound Identifier | Compound Identifier |
| 1 | PNU-69176E |
| 2 | CYD-1-79 |
| 3 | CTW0415 |
| 4 | VA012 |
| 5 | — |
| 6 | Oleamide |
| 7 | JPC0322-2 |
| 8 | CTW0497 |
| 9 | CTW0429 |
| 10 | CTW0410 |
| 11 | CTW0428 |
| 12 | CTW0411 |
| 13 | JPC0323 |
| 14 | JPC0314 |
| 15 | JPC0316 |
| 16 | JPC0376 |
| 17 | CTW0441 |
| 18 | CTW0442 |
| 19 | CTW0443 |
| 20 | CTW0462 |
| 21 | CTW0453 |
| 22 | CTW0452 |
| 23 | CTW0455 |
| 24 | CTW0498 |
| 25 | CTW0499 |
| 26 | EAW0120 |
| 27 | EAW0118 |
| 28 | CTW0427 |
| 29 | JPC0375 |
| 30 | JPC0377 |

### 4.0 Serotonin (e-HT) Receptors

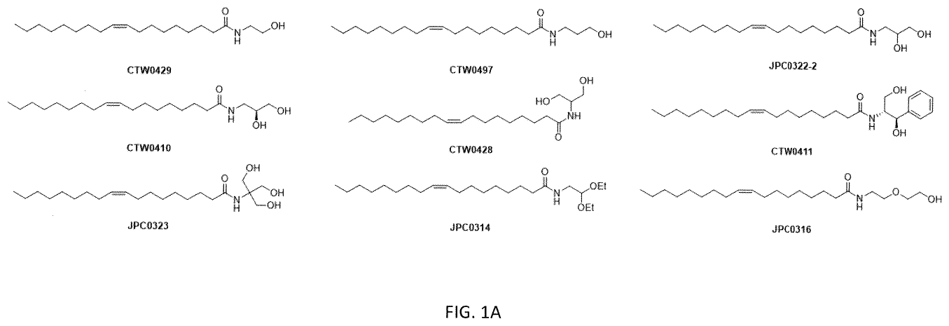

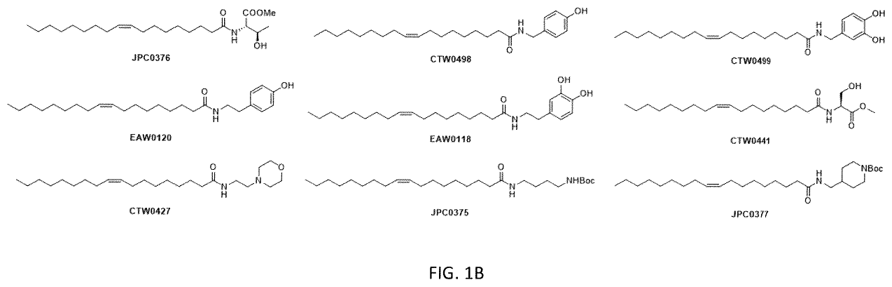

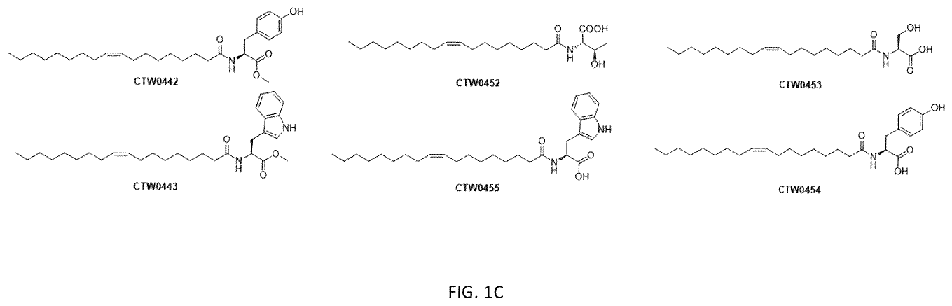

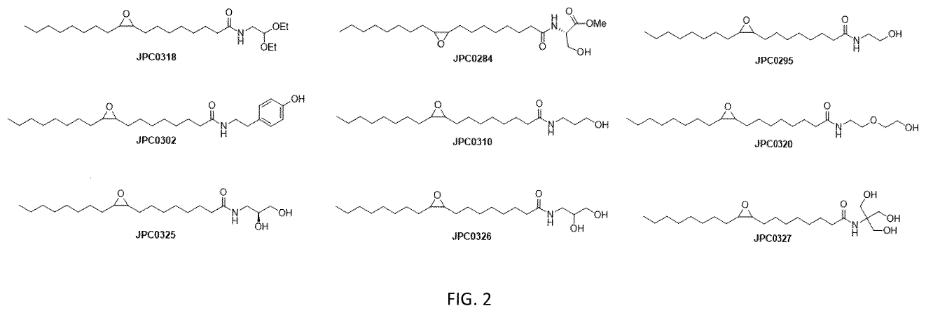

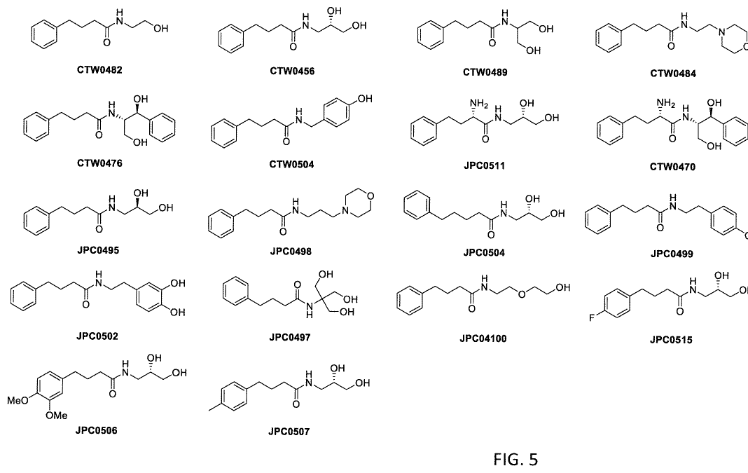

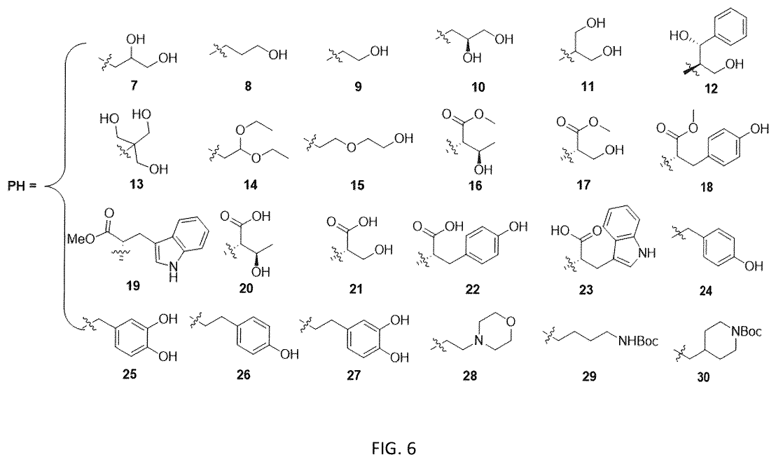

Structures for compounds 7 to 30 are shown in FIGS. 1A-1C, using the corresponding alphanumeric compound identifiers. The compounds in FIGS. 2-5 are shown with alphanumeric compound identifiers, but these compounds are not necessarily referred to herein with equivalent numeric identifiers. Substructures shown in FIG. 6 are numbered according to compounds 7 to 30, which include the substructures as substituents on the carboxamide nitrogen atom.

Fourteen serotonin (5-HT) receptors are identified with one ionotropic receptor (5-HT₃R), and 13 Class A G protein-coupled receptors (GPCRs) designated as 5-HT₁₋₇R based on structural and pharmacological criteria. Serotonin 5-HT₂ receptors (5-HT₂Rs) are a pharmacologically important 5-HT receptor family that includes the three subtypes 5-HT_{2A}R, 5-HT_{2B}R, and 5-HT_{2C}R, which share approximately 80% sequence homology in the transmembrane (TM) ligand-binding regions. Among them, the 5-HT_{2C}R and 5-HT_{2A}R have generated immense interest for pharmacologists and medicinal chemists in recent decades. The 5-HT_{2C}R and 5-HT_{2A}R are broadly distributed in the mammalian central nervous system (CNS) and mediate various brain functions including cognition, feeding, mood, learning, and memory.

The three 5-HT₂R subtypes (5-HT_{2A}R, 5-HT_{2B}R, and 5-HT_{2C}R) display similar molecular structures with a highly conserved endogenous agonist (5-HT) binding site, and intersecting signal transduction pathways and pharmacology. Traditional agonists have targeted the orthosteric ligand binding site within the seven-transmembrane bundle (7TM) of the receptor which is highly conserved. Allosteric modulators targeting a spatially and topographically distinct site may provide a useful pharmacological paradigm for GPCR drug discovery. The design of allosteric modulators that selectively target 5-HT₂R subtypes appears to be a viable and practical approach for avoiding ligand binding to other 5-HT receptors as well as the serotonin reuptake transporter, and is especially critical for avoiding 5-HT_{2B}R stimulation, which is thought to be associated with cardiac valvulopathy and pulmonary hypertension adverse effects.

### 5.1 Allosteric Modulators of 5-HT Receptors

Recent reports have described certain compounds as 5-HT₂R allosteric modulators, including, for example, 5-HT_{2C}R positive allosteric modulators (PAMs) 1 to 5. The complex natural product derivative compound 1 (PNU-69176E) was the first reported 5-HT_{2C}R PAM and was characterized by a stereo-dependent functional activity profile.

In earlier efforts to improve drug-like characteristics, analogs were prepared with variations on the α-D-galactopyranoside fragment, termed the polar head (“PH”) and the undecyl substituent at the 4-position of the piperidine, which is termed the lipophilic tail (“LT”). These efforts provided efficacious 5-HT_{2C}R PAMs 2 (CYD-1-79) and 3 (CTW0415). These 5-HT_{2C}R PAMs displayed improved pharmacokinetic (PK) profiles and demonstrated in vivo activity in preclinical animal models. Other recent efforts produced the N-benzyl-indole 4 (VA012) and piperazine-linked phenylcyclopropyl methanone 5 as 5-HT_{2C}R PAMs. Among them, compound 5 also exhibited 5-HT_{2B}R negative allosteric modulation (NAM) activity.

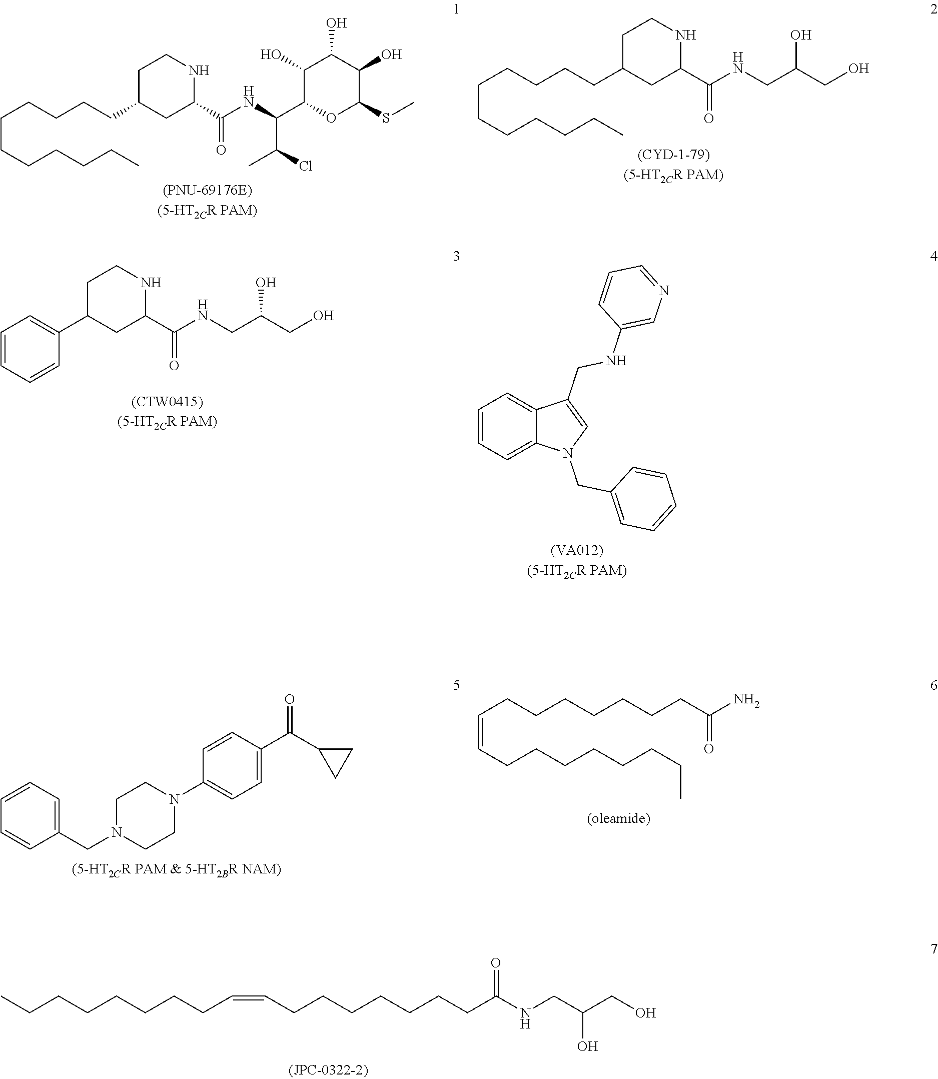

Compound 6 [oleamide, (Z)-9-octadecenamide-], an endogenous fatty acid amide, was identified in the cerebrospinal fluid of sleep-deprived cats as well as human plasma. Compound 6 is implicated in several biological and behavioral phenomena such as sleep induction, conditioned place aversion, feeding regulation, and hypothermia. Oleamide 6 was noted to act non-selectively as an agonist or allosteric modulator at the 5-HT_{1A}R, 5-HT_{2A}R, 5-HT_{2C}R and an inhibitor at 5-HT₇R as well as at other receptor systems. Compound 7 shares the common feature of a long LT and a terminal PH with the 5-HT_{2C}R PAM 2 (CYD-1-79). While the LT of compound 6 is longer than that of PAM 2 (18-carbon tail versus a 15-carbon tail respectively), an energy minimization overlay of compound 2 and compound 7 (an analog of compound 6 with a 1,2-diol PH fragment) suggested that, due to the cis conformation of the double bond, in some conformations the tail lengths may be similar in maximal length (17.98 Å vs. 17.78 Å).

### Compounds

In certain aspects, the present invention pertains to a compound of the Formula (I):

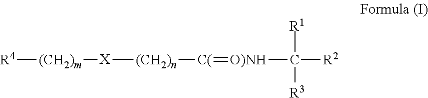

or a pharmaceutically acceptable salt thereof,
wherein:
R¹, R² and R³ are independently chosen from H, substituted or unsubstituted aryl, heteroaryl, arylalkyl, heteroaryl alkyl, carbonyl, C1 to C6 alkyl, C1 to C6 heteroalkyl, C1 to C6 alkoxy, C1 to C6 hydroxyalkyl, C1 to C6 ester, and heterocyclyl;
R⁴ is chosen from H, substituted or unsubstituted aryl, substituted or unsubstituted heteroaryl, arylalkyl and heteroaryl alkyl;
X is selected from the group consisting of: —CH₂CH₂—, —CH═CH—,

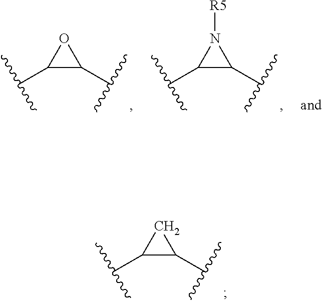

R⁵ is chosen from H, C1-C6 alkyl, and arylalkyl;
m is 0-20; and
n is 1-20.

In certain aspects, the present invention pertains to a compound of the Formula (Ia):

R⁴—(CH₂)ₘ—X—(CH₂)ₙ—C(═O)NH—(PH) Formula (Ia)
or a pharmaceutically acceptable salt thereof,
wherein:
R⁴ is chosen from H, substituted or unsubstituted aryl, substituted or unsubstituted heteroaryl, arylalkyl and heteroaryl alkyl;
X is selected from the group consisting of:
—CH₂CH₂—, —CH═CH—,

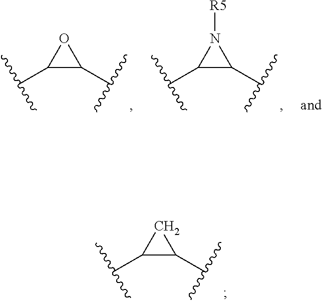

R⁵ is chosen from H, C1-C6 alkyl, and arylalkyl;
m is 0-20; and
n is 1-20;
and (PH) is any of the groups listed in FIG. 6.

In another aspect, the present invention pertains to a compound of the Formula (II):

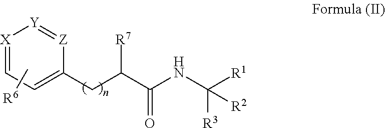

or a pharmaceutically acceptable salt thereof,
wherein R¹, R², R³ are independently chosen from H, substituted or unsubstituted aryl, heteroaryl, arylalkyl, heteroaryl alkyl, carbonyl, C1 to C6 alkyl, C1 to C6 heteroalkyl, C1 to C6 alkoxy, C1 to C6 hydroxyalkyl, and C1 to C6 ester;
R⁶ is chosen from H, NO₂, amino, CF₃, halogen, alkyl, or alkoxy;
R⁷ is H or NR⁸R⁹;
R⁸, R⁹ are independently chosen from H, alkyl, aryl, arylalkyl, cycloalkyl, alkoxy, and heteroalkyl, or
R⁸, R⁹ taken together with other atoms to form a 5- or 6-membered ring;
X, Y, Z are independently chosen from CH and N; and
n is 1 to 7.

In some embodiments, the invention pertains to a compound is chosen from Formula (II-(R)) (also referred to herein as Formula (IIa)) and Formula (II-(S)) (also referred to herein as Formula (IIb)):

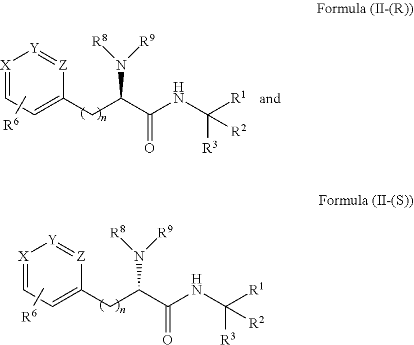

or a pharmaceutically acceptable salt thereof;
wherein the Formula (II-(R)) and Formula (II-(S)) designations refer to the respective R and S configurations at the chiral center carbon bonded to —NR⁸R⁹.

### Methods of Use

In some aspects, the present invention pertains to a method of treating a disease or condition, said method comprising administering to a patient a therapeutically effective amount of a compound of Formulas (I), (Ia), (II), (IIa), and (IIb), or a combination thereof, or a pharmaceutically acceptable salt thereof.

In some embodiments, treatment of the disease or condition involves modulation of 5-hydroxytryptamine 2A receptor and/or 5-hydroxytryptamine 2C receptor.

In some embodiments, the disease or condition may be treated by modulating 5-hydroxytryptamine 2A receptor and/or 5-hydroxytryptamine 2C receptor.

In some embodiments, the method involves the use of one or more compounds chosen from any of Formulas (I), (Ia), (II), (IIa), and (IIb), and combinations thereof, or a pharmaceutically acceptable salt thereof, to modulate 5-hydroxytryptamine 2A receptor and/or 5-hydroxytryptamine 2C receptor.

In some embodiments, said disease or condition is a substance use disorder, a psychiatric or neurological disorder, obesity, a mood disorder or a seizure disorder.

It is to be understood that both the foregoing descriptions are exemplary, and thus do not restrict the scope of the invention.

### EXAMPLES

The following examples are provided solely to illustrate the present invention and are not intended to limit the scope of the invention, described herein.

The description of preparation of certain compounds of the invention is meant to be exemplary of certain embodiments of the invention. The reagents and reactant used for synthetic conversions outlined herein and below is merely exemplary. The invention contemplates using the same or different reagents discussed herein to achieve preparation of the compounds of the invention.

### Example 1—Synthesis of Oleamide Analogs

Certain compounds of Formula I, such as oleamide analogs 7-30 may be prepared according to Scheme 1. An effective and convenient one-step coupling of oleic acid, a long-chain unsaturated omega-9 fatty acid, with various amino alcohol analogs, amino acid residues or monoamine-like fragments was conducted via adapting the common condensation reagent N,N,N′,N′-tetramethyl-O-(1H-benzotriazol-1-yl)uronium hexafluorophosphate (HBTU) or 1-ethyl-(3-dimethylaminopropyl)carbodiimide hydrochloride (EDCI) in combination with 1-hydroxybenzotriazole and the organic base N,N-diisopropylethylamine (DIPEA). Compounds 16-19 underwent saponification according to standard protocols to yield the corresponding carbonyl acid compounds 20-23. Compounds 7-30 were accomplished in 42-94% yields and then subjected to in vitro functional assessment.

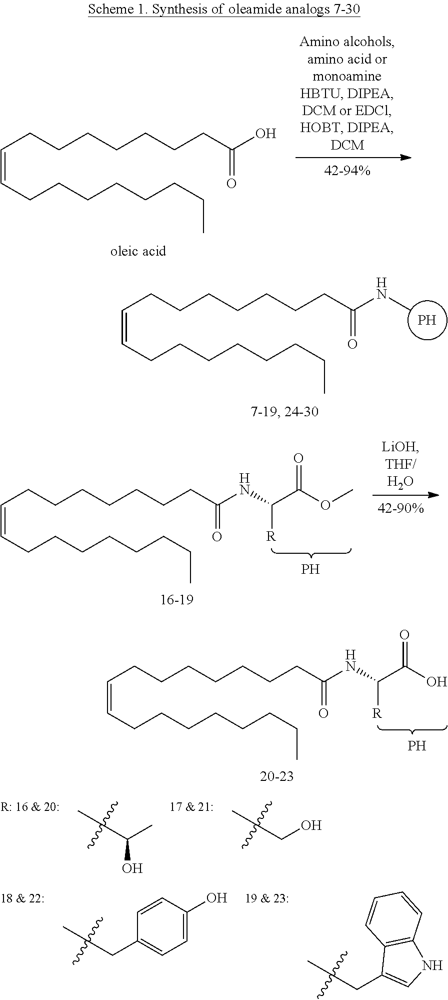

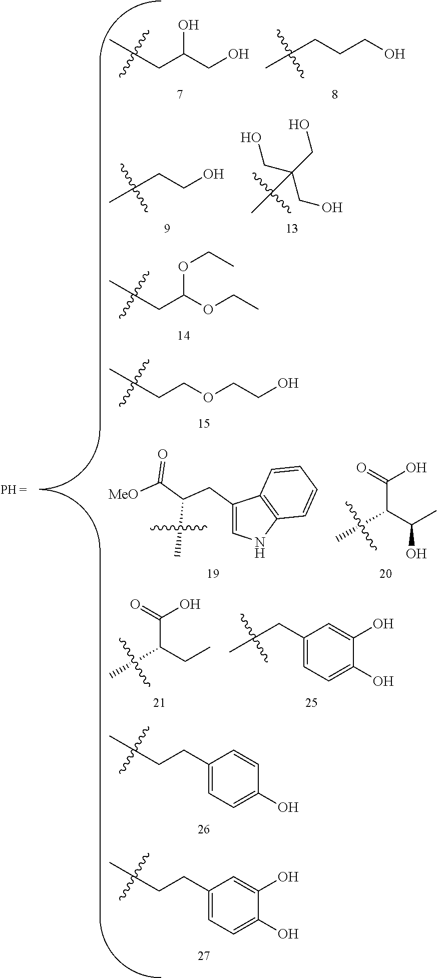

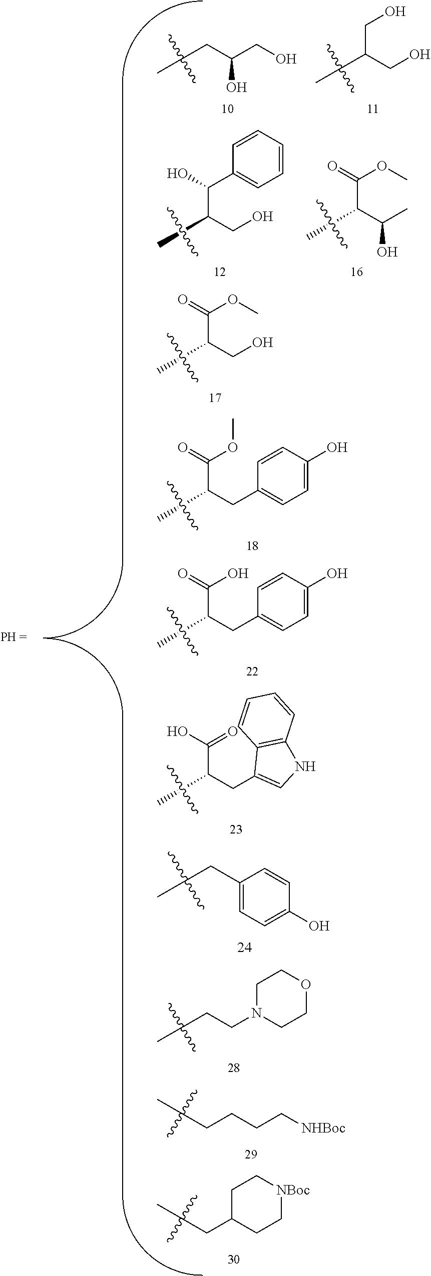

The reagents and reactants used for synthetic conversions outlined herein and below is merely exemplary. The invention contemplates using the same or different reagents discussed herein to achieve preparation of the compounds of the invention.

General. All commercially available reaction reagents and solvents were reagent grade and used directly. Preparative column chromatography was carried out using silica gel 60, particle size 0.063-0.200 mm (70-230 mesh, flash). Analytical TLC was performed employing silica gel 60 F254 plates (Merck, Darmstadt). NMR spectra were recorded on a Bruker-600 (¹H, 600 MHz; ¹³C, 150 MHz) spectrometer or Bruker-300 (OH, 300 MHz; ¹³C, 75 MHz). ¹H and ¹³C NMR spectra were recorded with tetramethylsilane (TMS) as an internal reference. Chemical shifts were presented in ppm, and J values were expressed in Hz. Melting points were obtained on a Thermo Scientific electrothermal digital melting point apparatus. High-resolution mass spectra (HRMS) were conducted with Thermo Fisher LTQ Orbitrap Elite mass spectrometer. Parameters are as the following: Nano ESI spray voltage was 1.8 kV; capillary temperature was 275° C., and the resolution was 60 000; ionization was achieved by positive mode. Purity of final compounds was carried out on a Shimadzu HPLC system (model CBM-20A LC-20AD SPD-20A UV/vis) with analytical conditions as following: Waters μBondapak C18 (300 mm×3.9 mm); flow rate 0.5 mL/min; UV detection at 254 and 210 nm; linear gradient from 30% acetonitrile in water (0.1% TFA) to 100% acetonitrile (0.1% TFA) in 20 min followed by 30 min of the last-named solvent. All newly synthesized compounds were characterized with ¹H NMR, ¹³C NMR, HRMS and HPLC analysis. All biologically evaluated compounds are >95% pure.

General procedure for the synthesis of compounds of the invention as exemplified by oleamide analogs 7-30.

To a solution of oleic acid (1.0 equiv) in a solvent such as dichloromethane (2 mL) was added HBTU (1.3 equiv) or EDCI (1.5 equiv) in combination with 1-hydroxybenzotriazole (1.5 equiv) and stirred under room temperature, then alcohol analogs, amino acid analogs, and monoamine-like fragments (1.1 equiv) along with DIPEA (2.5 equiv) were added to the solution. The reaction mixture was stirred for another 8 hours and TLC plate was used to detect the reaction with potassium permanganate chromogenic agent. After the completion of reaction, saturated ammonium chloride aqueous solution (10 mL) was titrated to the solution to quench the reaction and then the mixture system was extracted with ethyl acetate (20 mL×3) and washed with water, brine and then dried over anhydrous Na₂SO₄ and filtered. The organic solvent was concentrated under reduced pressure and purified on a silica gel column (DCM:MeOH=99:1) afforded the desired product 7-30.

N-(2,3-Dihydroxypropyl)oleamide (7): Compound 7 (57 mg, 80%) was prepared from oleic acid (0.20 mmol) followed the general synthetic procedure for 7-30, as a white wax-like material. ¹H NMR (300 MHz, CDCl₃) δ 6.13 (s, 1H), 5.36 (h, J=4.0 Hz, 2H), 3.77 (q, J=5.2 Hz, 1H), 3.57 (t, J=4.1 Hz, 2H), 3.42 (q, J=5.8 Hz, 2H), 2.23 (t, J=7.6 Hz, 2H), 2.02 (q, J=6.2 Hz, 4H), 1.64 (t, J=7.4 Hz, 2H), 1.30 (d, J=10.6 Hz, 20H), 0.89 (t, J=6.4 Hz, 3H). ¹³C NMR (75 MHz, CDCl₃) δ 175.3, 130.0, 129.7, 71.2, 63.6, 42.2, 36.6, 31.9, 29.8, 29.7, 29.5, 29.3, 29.2, 29.1, 27.22, 27.16, 25.7, 22.7, 14.1. HRMS (ESI) calcd for C₂₁H₄₁NO₃ [M+H]⁺ 356.3159; found 356.3158.

N-(3-Hydroxypropyl)oleamide (8): Compound 8 (25 mg, 52%) was prepared from oleic acid (0.14 mmol) followed the general synthetic procedure for 7-30, as a white solid, mp 63.0-63.5° C.; ¹H NMR (300 MHz, CDCl3) δ 5.91 (s, 1H), 5.43-5.30 (m, 2H), 3.64 (t, J=5.6 Hz, 2H), 3.46-3.37 (m, 2H), 2.20 (t, J=7.6 Hz, 2H), 2.02 (q, J=6.2 Hz, 4H), 1.66 (dt, J=14.6, 6.9 Hz, 4H), 1.42-1.18 (m, 20H), 0.95-0.82 (m, 3H). ¹³C NMR (75 MHz, CDCl₃/MeOD) δ 175.1, 129.9, 129.6, 59.1, 38.5, 36.5, 36.1, 36.0, 31.9, 31.8, 29.7, 29.6, 29.4, 29.23, 29.19, 29.1, 27.13, 27.10, 25.8, 22.6, 14.0. HRMS (ESI) calcd for C₂₁H₄₁NO₂ [M+H]⁺ 340.3210; found 340.3352.

N-(2-Hydroxyethyl)oleamide (9): Compound 9 (52 mg, 71%) was prepared from oleic acid (0.27 mmol) followed the general synthetic procedure for 7-30, as a white solid, mp 63.0-63.5° C.; ¹H NMR (300 MHz, CDCl₃) δ 6.34 (s, 1H), 5.41-5.26 (m, 2H), 3.70 (t, J=4.9 Hz, 2H), 3.56 (s, 1H), 3.40 (dd, J=10.2, 5.4 Hz, 2H), 2.24-2.15 (m, 2H), 2.06-1.95 (m, 4H), 1.73-1.51 (m, 2H), 1.29 (d, J=10.0 Hz, 20H), 0.88 (t, J=6.7 Hz, 3H). ¹³C NMR (75 MHz, CDCl₃) δ 174.6, 130.0, 129.7, 62.1, 42.4, 36.6, 31.9, 29.8, 29.7, 29.5, 29.31, 29.28, 29.2, 27.21, 27.17, 25.7, 22.7, 14.1. HRMS (ESI) calcd for C₂₀H₄₀NO₂ [M+H]⁺ 326.3054; found 326.3570.

(S)—N-(2,3-Dihydroxypropyl)oleamide (10): Compound 10 (32 mg, 60%) was prepared from oleic acid (0.15 mmol) followed the general synthetic procedure for 7-30, as a white wax-like material. ¹H NMR (300 MHz, CDCl₃) δ 6.44 (t, J=6.1 Hz, 1H), 5.41-5.29 (m, 2H), 3.98 (bs, 1H), 3.85 (s, 1H), 3.76 (t, J=5.2 Hz, 1H), 3.55 (bs, 2H), 3.40 (tq, J=14.1, 8.2, 6.8 Hz, 2H), 2.22 (t, J=7.6 Hz, 2H), 2.02 (q, J=6.4 Hz, 4H), 1.77-1.54 (m, 2H), 1.40-1.19 (m, 20H), 0.93-0.84 (m, 3H). ¹³C NMR (75 MHz, CDCl₃) δ 175.4, 130.0, 129.7, 71.1, 63.6, 42.1, 36.6, 31.9, 29.8, 29.7, 29.5, 29.32, 29.29, 29.2, 27.23, 27.18, 25.7, 22.7, 14.1. HRMS (ESI) calcd for C₂₁H₄₁NO₃ [M+H]⁺ 356.3159; found 356.3154.

N-(1,3-Dihydroxypropan-2-yl)oleamide (11): Compound 11 (58 mg, 60%) was prepared from oleic acid (0.27 mmol) followed the general synthetic procedure for 7-30, as a whitish wax. ¹H NMR (300 MHz, CDCl₃) δ 6.69 (d, J=7.9 Hz, 1H), 5.46-5.16 (m, 2H), 3.97-3.77 (m, 1H), 3.65 (ddd, J=28.2, 11.3, 4.8 Hz, 4H), 2.86 (s, 2H), 2.26-2.09 (m, 2H), 2.07-1.85 (m, 4H), 1.71-1.50 (m, 2H), 1.26 (d, J=9.6 Hz, 20H), 0.86 (t, J=6.6 Hz, 3H). ¹³C NMR (75 MHz, CDCl₃/CD₃OD) ¹³C NMR (75 MHz, CDCl₃) δ 175.0, 130.0, 129.7, 61.7, 52.4, 52.3, 36.6, 36.5, 31.8, 29.71, 29.69, 29.5, 29.3, 29.2, 29.1, 27.2, 27.1, 25.7, 22.6, 14.0. HRMS (ESI) calcd for C₂₁H₄₁NO₃ [M+H]⁺ 356.3159; found 356.3150.

N-((2S)-1,3-Dihydroxy-1-phenylpropan-2-yl)oleamide (12): Compound 12 (61 mg, 94%) was prepared from oleic acid (0.15 mmol) followed the general synthetic procedure for 7-30, as an off-white wax-like material. ¹H NMR (300 MHz, CDCl₃/CD₃OD) δ 7.41-7.14 (m, 5H), 6.68-6.77 (d, J=8.4 Hz, 1H), 5.39-5.25 (m, 2H), 4.97 (d, J=3.5 Hz, 1H), 4.08-3.99 (m, 1H), 3.89 (d, J=2.5 Hz, 2H), 3.63 (qd, J=11.1, 5.8 Hz, 2H), 2.08 (t, J=7.3 Hz, 2H), 1.99 (q, J=7.2 Hz, 4H), 1.44 (p, J=7.4 Hz, 2H), 1.26 (d, J=9.4 Hz, 20H), 0.92-0.78 (m, 3H). ¹³C NMR (75 MHz, CDCl₃/CD₃OD) δ 175.0, 141.5, 129.9, 129.7, 128.1, 127.4, 125.7, 71.8, 62.2, 56.4, 56.3, 36.5, 36.4, 31.8, 29.69, 29.66, 29.4, 29.24, 29.19, 29.1, 29.0, 27.1, 25.7, 22.6, 14.0. HRMS (ESI) calcd for C₂₇H₄₃NO₃ [M+H]⁺ 432.3472; found 432.3465.

N-(1,3-dihydroxy-2-(hydroxymethyl)propan-2-yl) (13): Compound 13 (73 mg, 94%) was prepared from oleic acid (0.20 mmol) followed the general synthetic procedure for 7-30, as a white wax. ¹H NMR (300 MHz, CDCl₃) δ 6.51 (s, 1H), 5.36 (s, 2H), 3.60 (s, 6H), 2.24 (t, J=7.6 Hz, 2H), 2.02 (d, J=6.1 Hz, 4H), 1.62 (s, 2H), 1.30 (d, J=11.4 Hz, 20H), 0.90 (d, J=6.0 Hz, 3H). ¹³C NMR (75 MHz, CDCl₃) δ 175.3, 130.0, 129.7, 61.8, 61.6, 37.0, 31.9, 29.7, 29.5, 29.3, 29.2, 29.1, 27.2, 25.8, 22.7, 14.1. HRMS (ESI) calcd for C₂₂H₄₄NO₄ [M+H]⁺ 386.3265; found 386.3262.

N-(2,2-diethoxyethyl)oleamide (14): Compound 14 (56 mg, 70%) was prepared from oleic acid (0.20 mmol) followed the general synthetic procedure for 7-30, as a colorless oil. ¹H NMR (300 MHz, CDCl₃) δ 5.78-5.65 (m, 1H), 5.41-5.25 (m, 2H), 4.50 (t, J=5.2 Hz, 1H), 3.70 (dq, J=9.6, 7.1 Hz, 2H), 3.53 (dq, J=15.9, 6.8 Hz, 2H), 3.38 (t, J=5.5 Hz, 2H), 2.17 (t, J=7.6 Hz, 2H), 2.00 (q, J=6.2 Hz, 4H), 1.62 (t, J=7.4 Hz, 2H), 1.42-1.14 (m, 26H), 0.88 (t, J=6.5 Hz, 3H). ¹³C NMR (75 MHz, CDCl₃) δ 173.2, 130.0, 129.7, 100.8, 62.8, 41.9, 36.7, 31.9, 29.73, 29.68, 29.5, 29.3, 29.2, 29.1, 27.2, 27.1, 25.7, 22.6, 15.3, 14.1. HRMS (ESI) calcd for C₂₄H₄₇NO₃Na [M+Na]⁺ 420.3448; found 420.3446.

N-(2-(2-hydroxyethoxy)ethyl)oleamide (15): Compound 15 (62 mg, 83%) was prepared from oleic acid (0.20 mmol) followed the general synthetic procedure for 7-30, as a colorless oil. ¹H NMR (300 MHz, CDCl₃) δ 6.21-6.03 (m, 1H), 5.33 (q, J=6.3 Hz, 2H), 3.82-3.69 (m, 2H), 3.57 (q, J=4.4 Hz, 4H), 3.46 (q, J=5.4 Hz, 2H), 2.59 (s, 1H), 2.18 (t, J=7.6 Hz, 2H), 2.01 (q, J=6.4 Hz, 4H), 1.63 (t, J=7.4 Hz, 2H), 1.29 (d, J=9.6 Hz, 20H), 0.88 (t, J=6.3 Hz, 3H). ¹³C NMR (75 MHz, CDCl₃) δ 173.5, 130.0, 129.7, 72.2, 70.0, 61.7, 39.2, 36.7, 31.9, 29.7, 29.5, 29.3, 29.1, 27.1, 25.7, 22.6, 14.1. HRMS (ESI) calcd for C₂₂H₄₄NO₃ [M+H]⁺ 370.3316; found 370.3314.

Methyl oleoyl-L-allothreoninate (16): Compound 16 (113 mg, 68%) was prepared from oleic acid (0.42 mmol) followed the general synthetic procedure for 7-30, as a white solid; mp 63.0-63.5° C. ¹H NMR (300 MHz, CDCl₃) δ 6.67-6.32 (m, 1H), 5.47-5.10 (m, 2H), 4.69-4.48 (m, 1H), 4.33 (s, 1H), 3.74 (s, 3H), 3.26 (s, 1H), 2.27 (t, J=7.6 Hz, 2H), 2.11-1.89 (m, 4H), 1.79-1.51 (m, 2H), 1.28 (d, J=11.6 Hz, 20H), 1.19 (d, J=6.4 Hz, 3H), 0.87 (t, J=6.6 Hz, 3H). ¹³C NMR (75 MHz, CDCl₃) δ 174.1, 171.7, 130.0, 129.7, 67.8, 57.3, 52.4, 38.6, 36.5, 31.9, 29.74, 29.71, 29.5, 29.29, 29.27, 29.24, 29.15, 27.19, 27.16, 25.7, 22.6, 20.0, 14.1. HRMS (ESI) calcd for C₂₃H₄₃NO₄ [M+H]⁺ 398.3265; found 398.3453.

Methyl oleoyl-L-serinate (17): Compound 17 (100 mg, 62%) was prepared from oleic acid (0.42 mmol) followed the general synthetic procedure for 7-30, as a colorless oil. ¹H NMR (300 MHz, CDCl₃) δ 6.62 (s, 1H), 5.44-5.18 (m, 2H), 4.77-4.54 (m, 1H), 4.08-3.82 (m, 2H), 3.78 (s, 3H), 3.36 (s, 1H), 2.33-2.15 (m, 2H), 2.12-1.91 (m, 4H), 1.78-1.51 (m, 2H), 1.28 (d, J=10.1 Hz, 20H), 0.88 (t, J=6.6 Hz, 3H). ¹³C NMR (75 MHz, CDCl₃) δ 173.9, 171.1, 130.0, 129.7, 63.2, 54.6, 52.7, 36.4, 31.9, 29.8, 29.7, 29.5, 29.30, 29.26, 29.2, 29.1, 27.21, 27.17, 25.6, 22.7, 14.1. HRMS (ESI) calcd for C₂₂H₄₁NO₄ [M+H]⁺ 384.3108; found 384.3457.

Methyloleoyl-L-tyrosinate (18): Compound 18 (143 mg, 74%) was prepared from oleic acid (0.42 mmol) followed the general synthetic procedure for 7-30, as a white solid; mp 71.5-72.3° C.; ¹H NMR (300 MHz, CDCl₃) δ 7.16 (s, 1H), 6.95 (d, J=8.5 Hz, 2H), 6.75 (d, J=8.5 Hz, 2H), 6.05 (d, J=8.0 Hz, 1H), 5.47-5.23 (m, 2H), 4.90 (dt, J=8.0, 6.0 Hz, 1H), 3.75 (s, 3H), 3.04 (ddd, J=30.8, 14.0, 5.9 Hz, 2H), 2.29-2.11 (m, 2H), 2.12-1.90 (m, 4H), 1.71-1.48 (m, 2H), 1.28 (s, 20H), 0.89 (t, J=6.7 Hz, 3H). ¹³C NMR (75 MHz, CDCl₃) δ 173.5, 172.5, 155.7, 130.2, 130.0, 129.8, 126.9, 115.6, 53.2, 52.4, 37.3, 36.6, 31.9, 29.8, 29.7, 29.5, 29.3, 29.2, 29.1, 27.23, 27.19, 25.6, 22.7, 14.1. HRMS (ESI) calcd for C₂₈H₄₅NO₄ [M+H]⁺ 460.3421; found 460.3436.

Methyl oleoyl-L-tryptophanate (19): Compound 19 (152 mg, 75%) was prepared from oleic acid (0.42 mmol) followed the general synthetic procedure for 7-30, as a yellow oil. ¹H NMR (300 MHz, CDCl₃) δ 8.43 (s, 1H), 7.56 (d, J=7.8 Hz, 1H), 7.37 (d, J=8.0 Hz, 1H), 7.17 (dtd, J=14.8, 7.1, 1.1 Hz, 2H), 6.98 (d, J=2.4 Hz, 1H), 6.03 (d, J=7.8 Hz, 1H), 5.49-5.24 (m, 2H), 5.00 (dt, J=7.9, 5.4 Hz, 1H), 3.71 (s, 3H), 3.34 (dd, J=5.3, 1.4 Hz, 2H), 2.22-2.11 (m, 2H), 2.03 (dd, J=7.8, 4.7 Hz, 4H), 1.69-1.50 (m, 2H), 1.30 (s, 20H), 0.91 (t, J=6.7 Hz, 3H). ¹³C NMR (75 MHz, CDCl₃) δ 172.9, 172.6, 136.2, 130.0, 129.8, 127.7, 122.7, 122.2, 119.6, 118.5, 111.3, 110.0, 52.9, 52.3, 36.6, 31.9, 29.8, 29.7, 29.5, 29.34, 29.26, 29.2, 29.1, 27.7, 27.3, 27.2, 25.5, 22.7, 14.1. HRMS (ESI) calcd for C₃₀H₄₆N₂O₃ [M+H]⁺ 483.3581; found 483.3417.

Oleoyl-L-allothreonine (20): Solid LiOH monohydrate (16.8 mg, 0.4 mmol) was added to a solution of 16 (40 mg, 0.10 mmol) in THF: H₂O; 3:1 (2 mL) at rt. The reaction mixture was stirred for 48 hrs and determined complete by TLC. The reaction mixture was neutralized with HCl and extracted with EtOAc (3×10 mL). The combined organic extracts were washed with brine (5 mL) and concentrated under reduced pressure to afford 20 (25 mg, 66%) as a colorless gel. ¹H NMR (600 MHz, CDCl₃) δ 6.99-6.71 (bs, 2H), 5.53-5.11 (m, 2H), 4.52 (d, J=6.9 Hz, 1H), 4.42 (s, 1H), 2.40-2.25 (m, 2H), 2.10-1.89 (m, 4H), 1.65 (s, 2H), 1.30 (d, J=18.5 Hz, 20H), 1.22 (d, J=5.2 Hz, 3H), 0.90 (t, J=6.9 Hz, 3H). ¹³C NMR (150 MHz, CDCl₃) δ 175.4, 174.3, 130.0, 129.6, 67.5, 57.7, 36.4, 31.9, 29.8, 29.6, 29.35, 29.32, 29.2, 27.24, 27.21, 25.8, 22.7, 19.4, 14.1. HRMS (ESI) calcd for C₂₂H₄₁NO₄ [M+H]⁺ 384.3108; found 384.3166.

Oleoyl-L-serine (21): Compound 21 (20 mg, 54%) was prepared from 17 by a procedure similar to that used to prepare compound 20, as a white wax-like material. ¹H NMR (300 MHz, CDCl₃) δ 5.31 (td, J=4.7, 2.1 Hz, 2H), 4.52 (t, J=3.8 Hz, 1H), 3.95 (dd, J=11.6, 3.9 Hz, 1H), 3.80 (dd, J=11.5, 3.7 Hz, 1H), 3.61 (s, 3H), 2.25 (dt, J=10.4, 7.5 Hz, 2H), 1.98 (q, J=6.3 Hz, 4H), 1.71-1.50 (m, 2H), 1.38-1.18 (m, 20H), 0.94-0.78 (m, 3H). ¹³C NMR (75 MHz, CDCl₃/MeOD) δ 174.4, 172.7, 130.0, 129.7, 62.6, 36.3, 31.9, 29.71, 29.68, 29.5, 29.3, 29.24, 29.20, 29.1, 27.2, 25.5, 22.6, 14.0. HRMS (ESI) calcd for C₂₁H₃₉NO₄ [M+H]⁺ 370.2952; found 370.2995.

Oleoyl-L-tyrosine (22): Compound 22 (40 mg, 90%) was prepared from 18 by a procedure similar to that used to prepare compound 20, as a white solid; mp 170.0-170.5° C. ¹H NMR (600 MHz, CDCl₃) δ 6.90 (d, J=8.0 Hz, 2H), 6.62 (d, J=8.0 Hz, 2H), 5.41-5.11 (m, 2H), 4.38 (s, 1H), 3.94-3.59 (bs, 1H), 3.02-2.91 (m, 1H), 2.90-2.74 (m, 1H), 2.04-1.93 (m, 6H), 1.44 (d, J=6.2 Hz, 2H), 1.36-1.10 (m, 20H), 0.85 (t, J=7.0 Hz, 3H). ¹³C NMR (150 MHz, CDCl₃/MeOD) δ 177.9, 174.3, 155.3, 130.2, 129.9, 129.7, 128.3, 115.3, 56.1, 37.0, 36.4, 31.9, 29.7, 29.5, 29.30, 29.27, 29.2, 27.2, 25.7, 22.6, 14.0. HRMS (ESI) calcd for C₂₇H₄₃NO₄ [M+H]⁺ 446.3265; found 446.3216.

Oleoyl-L-tryptophan (23): Compound 23 (22 mg, 42%) was prepared from 19 by a procedure similar to that used to prepare compound 20, as a white wax-like material. ¹H NMR (300 MHz, CDCl₃) δ 8.42 (s, 1H), 7.57 (d, J=7.8 Hz, 1H), 7.32 (d, J=8.0 Hz, 1H), 7.19 (t, J=7.2 Hz, 1H), 7.11 (t, J=7.2 Hz, 1H), 6.97 (s, 1H), 6.18 (d, J=7.6 Hz, 1H), 5.46-5.26 (m, 2H), 4.92 (dd, J=12.4, 5.4 Hz, 1H), 3.48-3.09 (m, 2H), 2.12-1.93 (m, 6H), 1.57-1.41 (m, 2H), 1.29 (s, 15H), 1.20 (s, 5H), 0.90 (t, J=6.7 Hz, 3H). ¹³C NMR (75 MHz, CDCl₃) δ 175.5, 174.3, 136.1, 130.0, 129.8, 127.8, 123.3, 122.1, 119.7, 118.4, 111.5, 109.5, 53.6, 36.4, 31.9, 29.8, 29.7, 29.6, 29.4, 29.3, 29.2, 29.1, 27.3, 27.2, 27.0, 25.4, 22.7, 14.1. HRMS (ESI) calcd for C9H44N₂O₃[M+H]⁺ 469.3425; found 469.3500.

N-(4-Hydroxybenzyl)oleamide (24): Compound 24 (40 mg, 69%) was prepared from oleic acid (0.15 mmol) followed the general synthetic procedure for 7-30, as a white solid; mp 71.5-72.3° C. ¹H NMR (300 MHz, CDCl₃) δ 7.85-7.67 (m, 1H), 7.14 (d, J=8.5 Hz, 2H), 6.86 (d, J=8.5 Hz, 2H), 6.03 (t, J=5.5 Hz, 1H), 5.61-5.25 (m, 2H), 4.39 (d, J=5.6 Hz, 2H), 2.37-2.18 (m, 2H), 2.06 (dd, J=7.9, 4.2 Hz, 4H), 1.79-1.61 (m, 2H), 1.34 (s, 20H), 0.95 (t, J=6.7 Hz, 3H). ¹³C NMR (75 MHz, CDCl₃) δ 173.8, 156.2, 130.0, 129.7, 129.20, 129.17, 115.8, 43.4, 36.8, 31.9, 29.8, 29.7, 29.5, 29.32, 29.25, 29.2, 29.1, 27.23, 27.18, 25.8, 22.7, 14.1. HRMS (ESI) calcd for C₂₅H₄₁NO₂ [M+H]⁺ 388.3210; found 388.3480.

N-(3,4-Dihydroxybenzyl)oleamide (25): Compound 25 (30 mg, 50%) was prepared from oleic acid (0.15 mmol) followed the general synthetic procedure for 7-30, as a colorless oil. ¹H NMR (300 MHz, CDCl₃) δ 6.93-6.79 (m, 2H), 6.67 (dd, J=8.1, 1.9 Hz, 1H), 6.17 (t, J=5.7 Hz, 1H), 5.59-5.24 (m, 2H), 4.34 (d, J=5.8 Hz, 2H), 2.34-2.21 (m, 2H), 2.15-1.95 (m, 4H), 1.81-1.55 (m, 2H), 1.34 (s, 20H), 0.95 (t, J=6.7 Hz, 3H). ¹³C NMR (75 MHz, CDCl₃) δ 174.4, 144.6, 144.3, 130.0, 129.8, 129.7, 119.7, 115.1, 114.9, 43.6, 36.8, 31.9, 29.8, 29.7, 29.5, 29.3, 29.22, 29.19, 29.1, 27.23, 27.17, 25.8, 22.7, 14.1. HRMS (ESI) calcd for C₂₅H₄₁NO₃ [M+H]⁺ 404.3159; found 404.3338.

N-(4-Hydroxyphenethyl)oleamide (26): Compound 26 (24 mg, 42%) was prepared from oleic acid (0.14 mmol) followed the general synthetic procedure for 7-30, as a white wax-like material. ¹H NMR (300 MHz, CDCl₃) δ 7.72 (s, 1H), 7.04 (d, J=8.5 Hz, 2H), 6.94-6.74 (m, 2H), 5.83-5.68 (m, 1H), 5.49-5.25 (m, 2H), 3.52 (q, J=6.9 Hz, 2H), 2.77 (t, J=7.0 Hz, 2H), 2.25-2.14 (m, 2H), 2.13-1.97 (m, 4H), 1.77-1.53 (m, 2H), 1.32 (s, 20H), 0.93 (t, J=6.7 Hz, 3H). ¹³C NMR (75 MHz, CDCl₃) δ 174.0, 155.4, 130.0, 129.7, 129.71, 129.66, 115.7, 41.0, 36.8, 34.8, 31.9, 29.8, 29.7, 29.5, 29.3, 29.2, 29.1, 27.23, 27.18, 25.8, 22.7, 14.1. HRMS (ESI) calcd for C₂₆H₄₃NO₂ [M+H]⁺ 402.3367; found 402.3293.

N-(3,4-Dihydroxyphenethyl)oleamide (27): Compound 27 (45 mg, 68%) was prepared from oleic acid (0.14 mmol) followed the general synthetic procedure for 7-30, as a white solid, mp 69.1-71.0° C.; ¹H NMR (300 MHz, CDCl₃) δ 7.94 (s, 1H), 6.85 (d, J=8.0 Hz, 1H), 6.79 (d, J=2.0 Hz, 1H), 6.59 (dd, J=8.0, 2.0 Hz, 1H), 5.86 (t, J=5.1 Hz, 1H), 5.49-5.25 (m, 2H), 3.51 (dd, J=13.1, 6.9 Hz, 2H), 2.72 (t, J=7.1 Hz, 2H), 2.29-2.14 (m, 2H), 2.14-1.97 (m, 4H), 1.75-1.52 (m, 2H), 1.32 (s, 20H), 0.93 (t, J=6.7 Hz, 3H). ¹³C NMR (75 MHz, CDCl₃) δ 174.6, 144.5, 143.3, 130.4, 130.0, 129.7, 120.4, 115.5, 115.3, 41.0, 36.8, 34.9, 31.9, 29.8, 29.7, 29.5, 29.33, 29.31, 29.21, 29.18, 29.1, 27.23, 27.18, 25.8, 22.7, 14.1. HRMS (ESI) calcd for C₂₆H₄₃NO₃ [M+H]⁺ 418.3316; found 418.3624.

N-(2-Morpholinoethyl)oleamide (28): Compound 28 (71 mg, 67%) was prepared from oleic acid (0.27 mmol) followed the general synthetic procedure for 7-30, as an off-white wax-like material. ¹H NMR (300 MHz, CDCl₃) δ 5.99 (s, 1H), 5.44-55.26 (m, 2H), 3.78-3.64 (m, 4H), 3.37 (q, J=6.0 Hz, 2H), 2.57-2.41 (m, 6H), 2.19 (t, J=6.0 Hz, 2H), 2.08-1.94 (m, 4H), 1.73-1.54 (m, 2H), 1.30 (d, J=11.9 Hz, 20H), 0.89 (t, J=6.7 Hz, 3H). ¹³C NMR (75 MHz, CDCl₃) δ 173.2, 130.0, 129.7, 66.9, 57.1, 53.3, 36.8, 35.5, 31.9, 29.8, 29.7, 29.5, 29.32, 29.29, 29.2, 27.22, 27.17, 25.8, 22.7, 14.1. HRMS (ESI) calcd for C₂₄H₄₆N₂O₂[M+H]⁺ 395.3632; found 395.3628.

tert-butyl(4-oleamidobutyl)carbamate (29): Compound 29 (73 mg, 81%) was prepared from oleic acid (0.20 mmol) followed the general synthetic procedure for 7-30, as a colorless oil. ¹H NMR (300 MHz, CDCl₃) δ 5.78 (s, 1H), 5.33 (d, J=5.3 Hz, 2H), 4.66 (s, 1H), 3.26 (q, J=6.2 Hz, 2H), 3.13 (d, J=6.4 Hz, 2H), 2.16 (t, J=7.6 Hz, 2H), 2.08-1.94 (m, 4H), 1.62 (t, J=7.5 Hz, 2H), 1.51 (d, J=4.5 Hz, 4H), 1.44 (s, 9H), 1.28 (d, J=9.2 Hz, 20H), 0.93-0.83 (m, 3H). ¹³C NMR (75 MHz, CDCl₃) δ 173.2, 156.1, 130.0, 129.7, 40.1, 39.0, 36.8, 31.9, 29.74, 29.70, 29.5, 29.29, 29.25, 29.1, 28.4, 27.6, 27.20, 27.16, 26.7, 25.8, 22.7, 14.1. HRMS (ESI) calcd for C₂₇H₅₃N₂O₃ [M+H]⁺ 453.4051; found 453.4059.

tert-butyl 4-(oleamidomethyl)piperidine-1-carboxylate (30): Compound 30 (81 mg, 85%) was prepared from oleic acid (0.20 mmol) followed the general synthetic procedure for 7-30, as a colorless oil. ¹H NMR (300 MHz, CDCl₃) δ 5.59 (t, J=6.1 Hz, 1H), 5.34 (td, J=7.5, 4.7 Hz, 2H), 4.20-4.02 (m, 2H), 3.15 (s, 2H), 2.68 (t, J=12.8 Hz, 2H), 2.18 (t, J=7.6 Hz, 2H), 2.02 (q, J=6.7 Hz, 4H), 1.65 (tt, J=8.3, 4.3 Hz, 5H), 1.46 (s, 9H), 1.29 (d, J=9.9 Hz, 20H), 1.19-1.09 (m, 2H), 0.89 (t, J=6.5 Hz, 3H). ¹³C NMR (75 MHz, CDCl₃) δ 173.3, 154.8, 130.0, 129.7, 79.4, 44.8, 43.6, 36.9, 36.4, 31.9, 29.8, 29.7, 29.5, 29.3, 29.24, 29.17, 29.13, 29.10, 28.4, 27.21, 27.16, 25.8, 22.7, 14.1. HRMS (ESI) calcd for C29H55N₂O₃[M+H]⁺ 479.4207; found 479.4216.

### Example 2: 5-HT-Evoked Intracellular Calcium (Ca²⁺) Release

A fluorescence-based assay was employed to measure 5-HT-evoked Caᵢ²⁺ levels as a measure of receptor activity in Chinese hamster ovary (CHO) cells stably transfected with the unedited (INI) isoform of the human (h) h5-HT_{2C}R (h5-HT_{2C}R-CHO cells), h5-HT_{2A}R (h5-HT_{2A}R-CHO cells), or h5-HT_{2B}R (h5-HT₂AR-CHO cells). The capacity of 5-HT to promote Caᵢ²⁺ release (Eₘₐₓ) was established and set as the 100% response.

The assays were conducted with Chinese hamster ovary (CHO) cells stably transfected with the human unedited (INI) h5-HT_{2C}R (h5-HT_{2C}R-CHO cells) or the human h5-HT_{2A}R (h5-HT_{2A}R-CHO cells), or the h5-HT_{2B}R (h5-HT₂AR-CHO cells), which were the generous gift from Drs. Kelly A. Berg and William P. Clarke (University of Texas Health Science Center, San Antonio, TX). The h5-HT_{2B}R (CHO-K1/5-HT_{2B}R; h5-HT₂AR-CHO cells) were purchased from GenScript, Piscataway, NJ. The cellular growth environment was as follows: 37° C., 5% CO₂, and 85% relative humidity. The h5-HT_{2C}R-CHO cells and h5-HT_{2A}R-CHO cells were cultured in GlutaMax-MEM medium (Invitrogen, Carlsbad, CA) containing 5% fetal bovine serum (Atlanta Biologicals, Atlanta, GA) and 100 μg/mL hygromycin (Mediatech, Manassas, VA). The h5-HT_{2B}R-CHO cells were cultured in Ham's F12 media supplemented with 10% FBS and 200 μg/ml Zeocin (Thermo Fisher Scientific, Carlsbad, CA). All cells were passaged when they reached 80% confluency.

The Caᵢ²⁺ release assay was performed according to the following procedure. Specifically, cells (150 μL; passages 9-15) were plated in serum-replete medium at a density of 14 000-16 000 (FlexStation 3; Molecular Devices) or 30 000 cells/well (FLIPR^{TETRA}; Molecular Devices) in black-wall 96-well culture plates with optically clear flat bottoms. After ˜24 hours, the medium was replaced with serum-free (SF) GlutaMax-MEM medium (h5-HT_{2C}R-CHO cells and h5-HT_{2A}R-CHO cells) or serum free HAM's F12 (h5-HT_{2B}R-CHO cells) supplemented with 20 nM to 100 μM putrescine (Sigma-Aldrich, St. Louis, MO), 20 nM to 100 μM progesterone (Sigma-Aldrich), and 1:100 ITS (1000 mg/L human recombinant insulin, 550 mg/L human recombinant transferrin, 0.67 mg/L selenious acid; Corning Inc., Corning, NY) (SF+ medium). After an incubation for another 3 h, SF+ medium for the h5-HT_{2C}R-CHO cells and h5-HT_{2A}R-CHO cells was replaced with 40 μL of Hank's balanced saline solution (HBSS; without CaCl₂) or MgCl₂, pH 7.4) plus 40 μL of Calcium 6 dye solution (FLIPR No-wash kit, Molecular Devices, Sunnyvale CA) supplemented with 2.5 mM of water-soluble probenecid (Sigma-Aldrich), and then the plate was incubated with dye solution in the dark for 2 h at 37° C. followed by 15 min at room temperature. For h5-HT₂AR-CHO cells, Calcium 6 dye was incubated in the presence of Serum free Ham's F12 medium supplemented with progesterone, putrescine and ITS as described above. The drug was diluted at 5× concentration in 1×HBSS and controls contained the same final concentration of diluent. The delivery of the compound (20 μL/well) was 15 min prior to the addition of 5-HT (10 μM to 100 μM; 25 μL/well), and a baseline was established for each well before the addition of the compound and 5-HT. The fluorescence readings were then adopted to evaluate the allosteric modulation of 5-HT-induced Caᵢ²⁺ release. FlexStation 3 (Molecular Device) or FLIPR^{TETRA} (80-130 gain, 80% intensity, 0.3s exposure) was used to measure fluorescence. For FlexStation 3, a 17 s baseline was established before the compound was added, and fluorescence was recorded every 1.7 s thereafter for 240 s. The maximum peak height of each well was determined by SoftMax software (Pro 5.4.5). For FLIPR^{TETRA}, a 10 s baseline was established before adding the compound, and then record the fluorescence every 1 s for 120 s after the compound or 360 s after 5-HT. The maximum peak height of each well was determined by ScreenWorks 4.0 software. After the final reading, the cells were fixed in 2% paraformaldehyde (Sigma) overnight. A 4-parameter nonlinear regression analysis (GraphPad Prism 7) was used to determine the 5-HT-induced Caᵢ²⁺ maximum release (Eₘₐₓ) in the presence of the test compound, and calculated from 4-6 biological replicates, each biological replicate performed in technical triplicates. The Eₘₐₓ of the test compound plus 5-HT was normalized to the Eₘₐₓ of 5-HT alone. Subsequently, Welch's unpaired t-test (GraphPad prism) was used for post hoc comparison of the Eₘₐₓ means. All statistical analyses were performed with an experimental error rate of α=0.05. All treatment assignments were blinded to investigators who performed in vitro assays and endpoint statistical analyses.

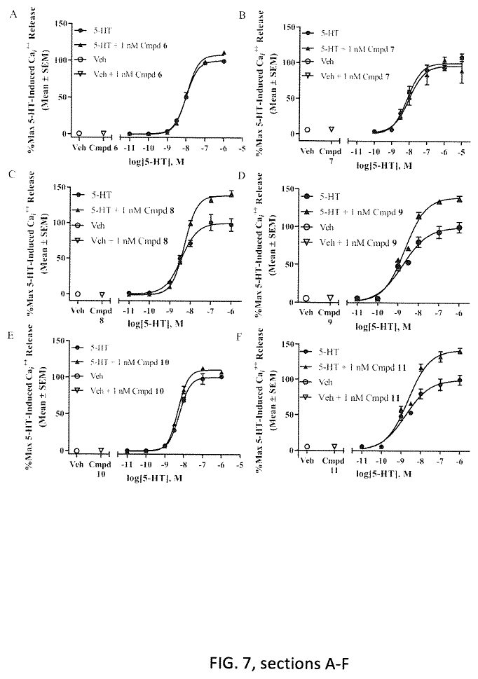

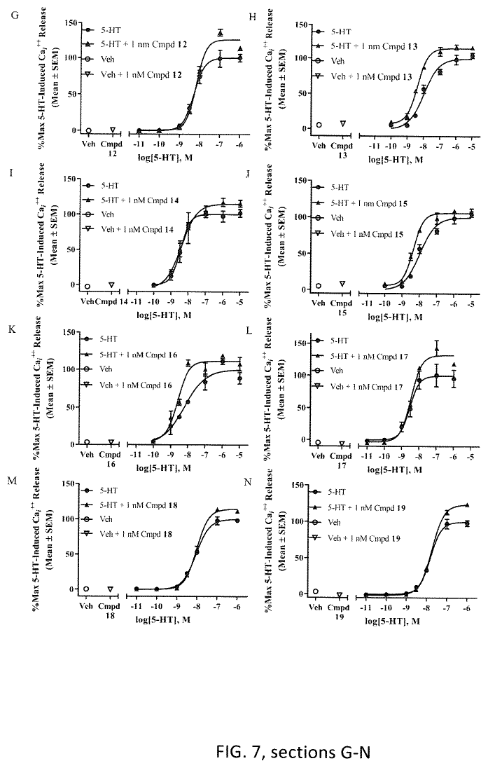

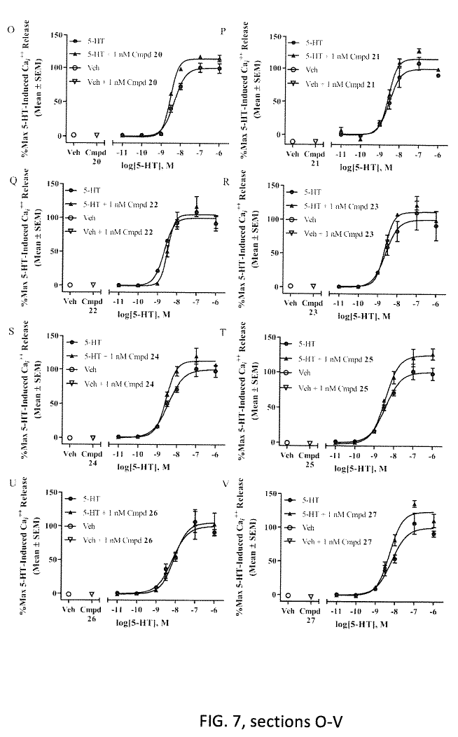

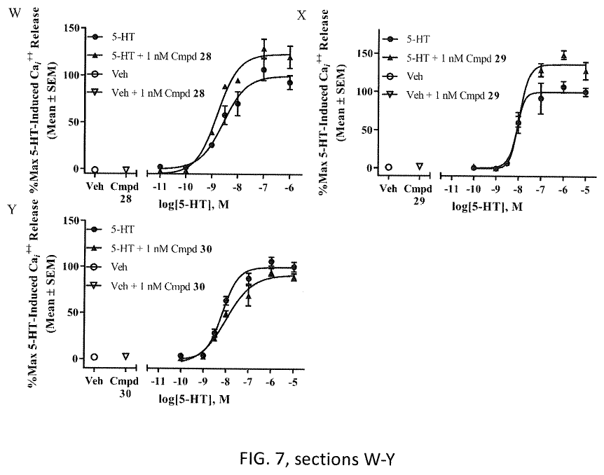

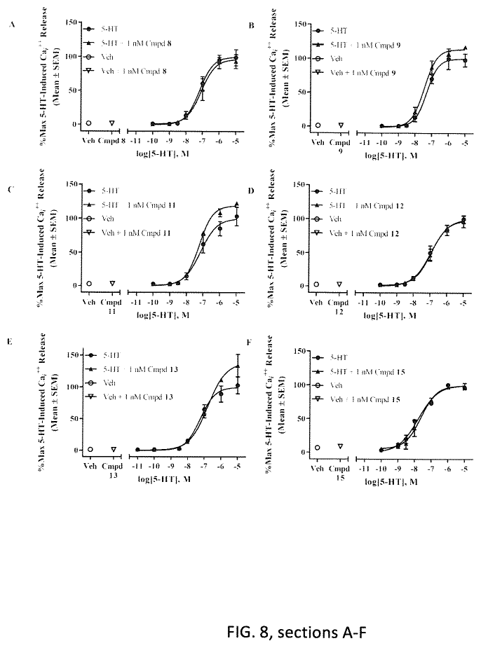

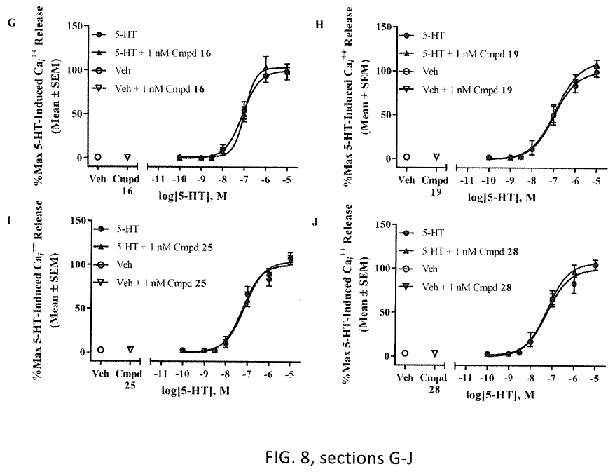

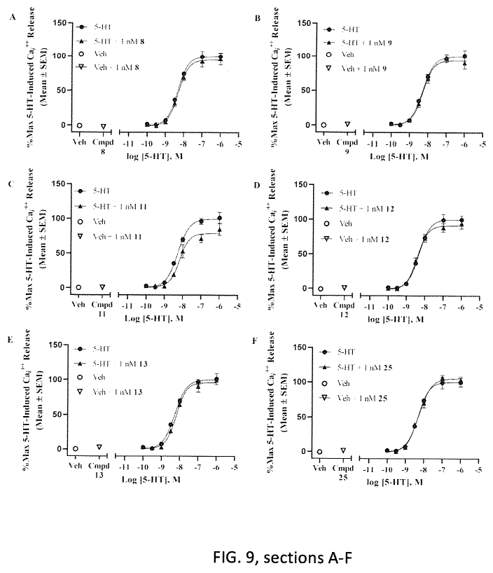

Previous studies demonstrated that the 5-HT_{2C}R PAM 2 at the concentration of 1 nM evoked ˜23% upward regulation of 5-HT-evoked Caᵢ²⁺ release. Thus, compounds 7-30 were screened at 1 nM in the presence of an increasing concentration of 5-HT to assess enhancement of 5-HT-induced Caᵢ²⁺ release (5-HT Eₘₐₓ). At 1 nM, none of the tested compounds exhibited intrinsic agonist activity to induce Caᵢ²⁺ release in h5-HT_{2C}R-CHO, h5-HT_{2A}R-CHO, or h5-HT_{2B}R-CHO cells when administered 15 min prior to the addition of 5-HT (for details see FIG. 7, sections A-Y; FIG. 8, sections A-J; FIG. 9, sections A-F).

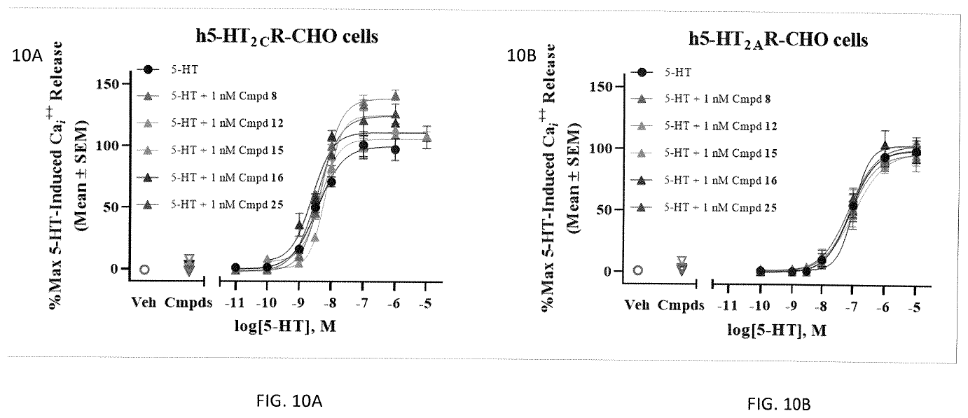

Without wishing to be limited by any particular theory, certain PH fragments may aid in forming interactions with the ECL2 and certain TM helices of the receptor. Compounds 7-30 with varied PHs were screened in vitro in h5-HT_{2C}R-CHO cells (Table 1). Oleamide (6) exhibited moderate enhancement (˜11%) of 5-HT-evoked Caᵢ²⁺ release under the test conditions while 7, which possesses the same 1,2-diol PH as 2 and 3, did not significantly increase 5-HT_{2C}R-evoked Caᵢ²⁺ (Table 1; FIG. 7). Exclusion of the middle hydroxyl group of 7 afforded the less-bulky oleyl propanolamide 8 which potentiated 5-HT-evoked Caᵢ²⁺ release (Table 1; FIG. 10A). Compound 9 with ethanolamide PH maintained 5-HT_{2C}R PAM activity (Table 1; FIG. 1A).

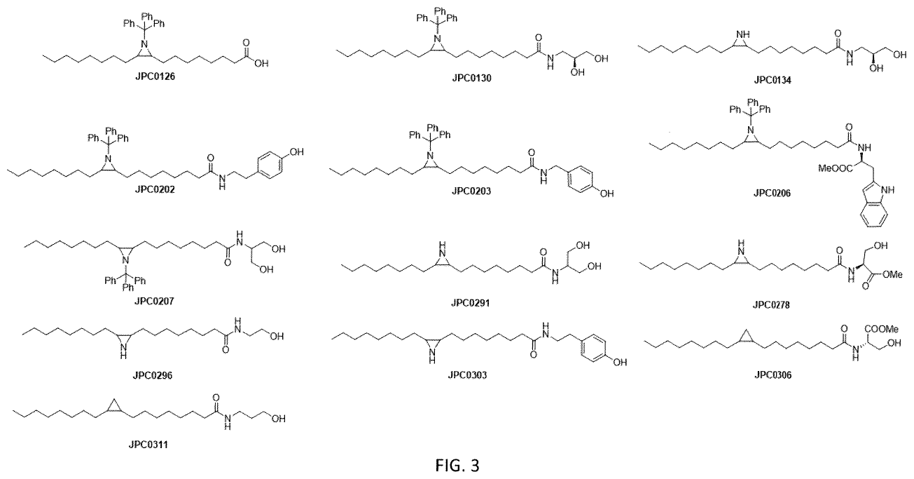

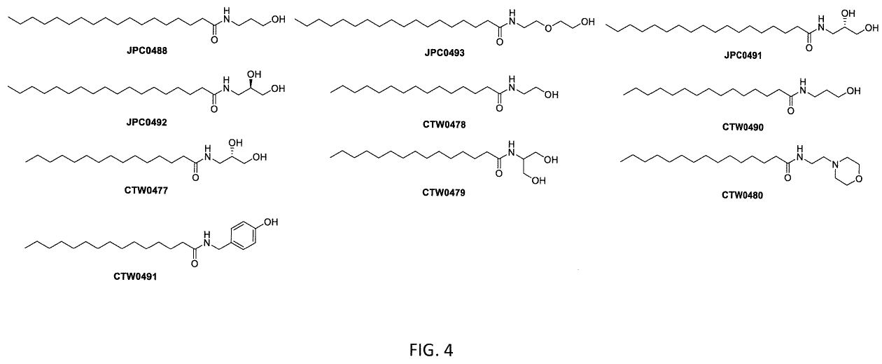

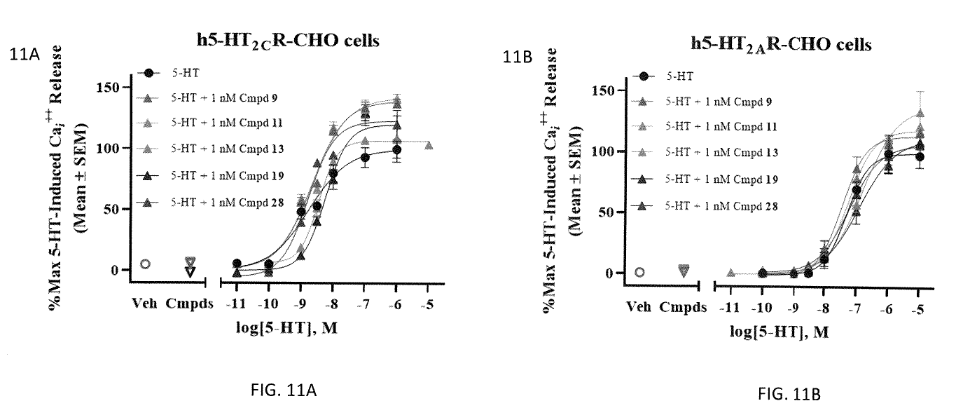

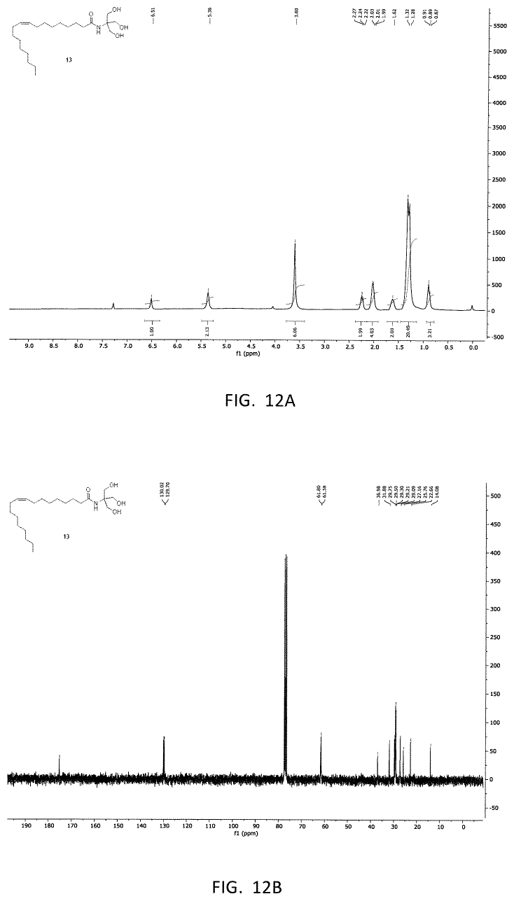

Since compound 2 exhibited a stereo conformation preference for 5-HT_{2C}R PAM activity, compound 10, furnished by introducing an S configurational 1,2-diol to the amide, was then explored and identified as less active (Table 1), suggesting the 1,2-diol PH of 2 and 3 was less favorable for the oleamide derivatives to produce 5-HT_{2C}R PAM activity. Compound 11, which has a 1,3-diol moiety versus a 1,2-diol PH, promoted a ˜44% increase in 5-HT_{2C}R-evoked Caᵢ²⁺ (Table 1; FIG. 4A). Compound 12, which incorporates a phenyl into the diol PH of 11, maintained 5-HT_{2C}R PAM activity (Table 1; FIG. 10A) as does compound 13 (Table 1; FIG. 11A) with one more hydroxymethyl group to 11. Compound 14 includes the hydroxyl group with ether did not evoke PAM activity, perhaps due to the loss of the terminal H-bond donor and the bulkier volume of the PH (Table 1). Meanwhile, lengthening the PH of 9 by etherification with another ethanol fragment was less favorable (15, FIG. 3A). These findings suggested that hydroxyl containing moiety may be important for 5-HT_{2C}R allosteric potentiation, and a proper size of the PH may be helpful for 5-HT_{2C}R PAM activity.

Regarding several other novel compounds according to the present invention, the chiral amino acids (16-23), terminal phenol (24 and 26), catechol (25 and 27), morpholino (28) or amino (29-30) substituted alkylamine exhibited mixed properties, including inactive or allosteric potentiation of 5-HT at the 5-HT_{2C}R (Table 1). As the introduction of a hydroxyl-containing moiety could potentiate 5-HT_{2C}R PAM activity, hydroxyl or terminal phenol containing chiral amino acids were applied by condensation of the methyl ester of threonine, serine, and tyrosine affording compounds 16-18. Among them, compound 16 (Table 1; FIG. 10A) demonstrated 5-HT_{2C}R PAM activity. Compound 19 (Table 1; FIG. 11A), incorporating the 5-HT indole-like tryptophan methyl ester in the terminal position of the PH, resulted in a 5-HT-evoked F. of 125.4% (FIG. 11A). Saponification of the methyl esters provided free acids 20-23. However, these carbonyl acid derivatives 20-23 at 1 nM did not potentiate 5-HT efficacy (Table 1). Furthermore, both 4-aminoalkyl phenolic and catechol moieties were explored as potential PHs (24-27). Terminal phenol compound 25 with a 4-aminomethyl catechol PH promoted 5-HT_{2C}R PAM activity (Table 1; FIG. 10A) while other terminal phenol compounds with less phenolic hydroxyl or more carbon spaced chain did not display 5-HT_{2C}R PAM efficacy (the 4-aminoalkyl phenolic 24, 26 and two-carbon spaced catechol 27, Table 1). When aliphatic amines were employed as PHs, the two-carbon spaced morpholino compound (28) displayed 5-HT_{2C}R PAM activity (Table 1; FIG. 11A), while the n-butylamine compound (29) with a bulky amine terminus did not induce a 5-HT_{2C}R PAM effect (Table 1). Surprisingly, when 4-(aminomethyl)piperidine was incorporated at the end of oleamide, a comparable ˜10% decrease in 5-HT_{2C}R-mediated Caᵢ²⁺ release was observed, which may signal the potential for 30 to function as a negative allosteric modulator (NAM) (Table 1).

TABLE 1
Effects of oleamide analogs 7-30 (1 nM) on 5-HT-induced Caᵢ²⁺ release in
h5-HT_{2C}R-CHO cells

| | | Eₘₐₓ RFU |
|---|---|---|
| Compound | PH | (% 5-HT)^{a} |
| 5-HT | | 100.0% |
| 6 | H | 111.4 ± 4.3* |
| 7 | 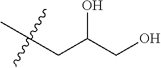 [SMILES: *CC(O)CO] | 108.0 ± 6.7 |
| 8 | 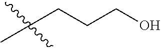 [SMILES: *CCCO] | 126.3 ± 8.5* |
| 9 | 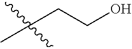 [SMILES: *CCO] | 124.0 ± 6.8* |
| 10 | 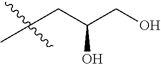 [SMILES: *C[C@H](O)CO] | 100.1 ± 8.8 |
| 11 | 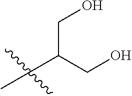 [SMILES: *C(CO)CO] | 143.9 ± 14.1* |
| 12 | 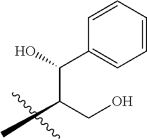 | 126.0 ± 6.9* |
| 13 | 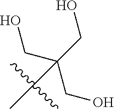 [SMILES: *C(CO)(CO)CO] | 113.2 ± 3.7* |
| 14 | 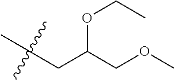 [SMILES: *CC(COC)OCC] | 111.1 ± 5.4 |
| 15 | 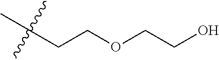 [SMILES: *CCOCCO] | 106.2 ± 2.1* |
| 16 | 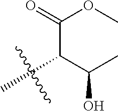 | 109.1 ± 2.0* |
| 17 | 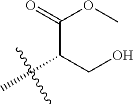 | 126.8 ± 11.4 |
| 18 | 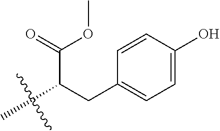 | 127.8 ± 15.3 |
| 19 | 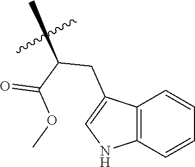 | 125.4 ± 5.2* |
| 20 | 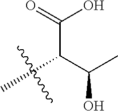 | 111.9 ± 4.4 |
| 21 | 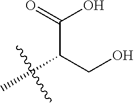 | 117.8 ± 6.9 |
| 22 | 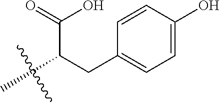 | 106.6 ± 9.0 |
| 23 | 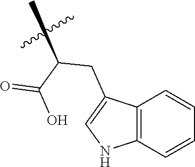 | 117.4 ± 12.4 |
| 24 | 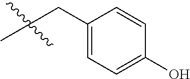 [SMILES: *Cc1ccc(O)cc1] | 111.9 ± 11.8 |
| 25 | 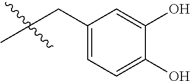 [SMILES: *Cc1ccc(O)c(O)c1] | 122.8 ± 6.7* |
| 26 | 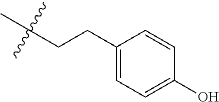 [SMILES: *CCc1ccc(O)cc1] | 116.0 ± 18.1 |
| 27 | 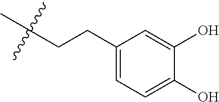 [SMILES: *CCc1ccc(O)c(O)c1] | 120.6 ± 15.5 |
| 28 | 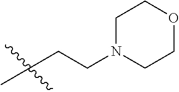 [SMILES: *CCN1CCOCC1] | 121.4 ± 5.2* |
| 29 | 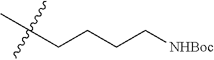 [SMILES: *CCCCNC(=O)OC(C)(C)C] | 118.5 ± 6.4 |
| 30 | 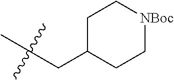 [SMILES: *CC1CCN(C(=O)OC(C)(C)C)CC1] | 92.9 ± 1.3* |

^{a}Addition of the synthetic compound (1 nM) occurred 15 min prior to assessment of Caᵢ²⁺ release evoked by increasing concentrations of 5-HT (vehicle, 10⁻¹¹ to 10⁻⁶ M) in h5-HT_{2C}R-CHO cells. Data are presented as maximal 5-HT-induced Caᵢ²⁺ release (Eₘₐₓ). *p <0.05. Comparisons between means for Eₘₐₓ were conducted with an unpaired t-test with Welch's correction (GraphPad Prism). All statistical analyses were conducted with an experiment-wise error rate of α = 0.05

Regarding FIGS. 10A and 10B, concentration-response curves are shown for compounds 8, 12, 15, 16, and 25 for 5-HT-induced Caᵢ²⁺ release in (10A) h5-HT_{2C}R-CHO cells or (10B) h5-HT_{2A}R-CHO cells. Representative curves show the concentration-response curve for 5-HT in the absence (black circles) and in the presence (closed triangles) of the test compounds; vehicle (open circle); test compound assessed alone (open triangle). The maximum 5-HT-induced Caᵢ²⁺ release in the absence of the test compounds was set as 100% and the F. of the test compounds were as listed in Table 1 and Table 2.

### Example 3: 5-HT-Induced Caᵢ²⁺ Release

A subset of the 10 oleamide-like compounds characterized as 5-HT_{2C}R PAMs (Table 1) were further evaluated in the in vitro Ca²⁺ efflux assay using h5-HT_{2A}R-CHO cells (Table 2). After screening of these 10 analogs at 1 nM in the h5-HT_{2A}R-CHO cells, compounds with two pharmacological profiles were identified. Compounds 8, 12, 15, 16, and 25 which were identified as 5-HT_{2C}R PAMs (Table 1; FIG. 10A) exhibited no efficacy as allosteric modulators of the 5-HT_{2A}R (Table 2; FIG. 10B). However, compounds 9, 11, 13, 19, and 28 identified as 5-HT_{2C}R PAMs (Table 1; FIG. 11A) exhibited efficacy as 5-HT_{2A}R PAMs (Table 2; FIG. 11B). Thus, 5-HT_{2C}R PAMs (8, 12, 15, 16, and 25) and dual 5-HT_{2C}R/5-HT_{2A}R PAMs (9, 11, 13, 19, and 28) were differentiated within this current series of oleamide-like compounds.

Compound 9, which possesses a hydroxyethyl PH, displayed 5-HT_{2A}R PAM activity (Table 2; FIG. 11B). Compounds 11 and 13, introducing a second and a third hydroxymethyl group to the PH of 9, respectively, maintained the 5-HT_{2A}R allosteric effect (Table 2; FIG. 11B). This result suggested that incorporation of two-carbon spaced hydroxyl moiety may be favorable for producing 5-HT_{2A}R PAM activity. Compound 19 bearing a methyl tryptophan as PH and compound 28 with a two-carbon linked morpholino PH also acted as 5-HT_{2A}R PAMs (Table 2; FIG. 11B). Neither lengthening the linker length of 9 by one more carbon (8), etherification with another ethanol fragment (15) nor retaining the 1,3-diol moiety of 11 with the addition of a phenyl (12) resulted in 5-HT_{2A}R PAM activity (Table 2; FIG. 11A). Compound 16 and 25 with longer PHs as methyl threonine and 4-aminomethyl catechol did not result in 5-HT_{2A}R PAM activity (Table 2). These findings suggested that the structure of 5-HT_{2A}R PAMs may be more sensitive to the length and volume change of PH than 5-HT_{2C}R PAMs (Tables 1-2). Representative 5-HT_{2C}R PAMs 8, 12, 25 and dual 5-HT_{2C}R/5-HT_{2A}R PAMs 9, 11, 13 were assessed in the in vitro functional assay in h5-HT_{2B}R-CHO cells (Table 3; FIG. 9). None of the compounds showed altered 5-HT_{2B}R-evoked Caᵢ²⁺ alone or in the presence of 5-HT (Table 3).

TABLE 2
Effects of identified oleamide-like 5-HT_{2C}R PAMs (1 nM) on 5-HT-
induced Caᵢ²⁺ release in h5-HT_{2A}R-CHO cells

| | | Eₘₐₓ RFU |
|---|---|---|
| Compound | PH | (% 5-HT)^{a} |
| 5-HT | | 100.0% |
| 8 | 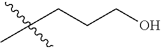 [SMILES: *CCCO] | 99.2 ± 2.6 |
| 9 | 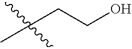 [SMILES: *CCO] | 121.0 ± 3.7* |
| 11 | 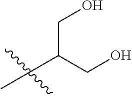 [SMILES: *C(CO)CO] | 119.0 ± 3.2* |
| 12 | 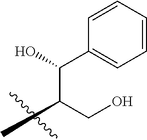 | 98.8 ± 3.8 |
| 13 | 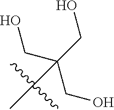 [SMILES: *C(CO)(CO)CO] | 132.4 ± 6.1* |
| 15 | 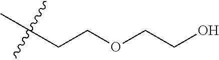 [SMILES: *CCOCCO] | 99.6 ± 0.6 |
| 16 | 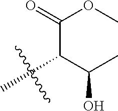 | 106.0 ± 5.3 |
| 19 | 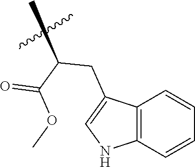 | 109.2 ± 3.5* |
| 25 | 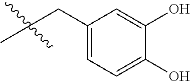 [SMILES: *Cc1ccc(O)c(O)c1] | 102.5 ± 2.7 |
| 28 | 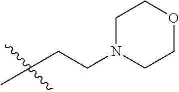 [SMILES: *CCN1CCOCC1] | 105.7 ± 2.0* |

ªAddition of the synthetic compound (1 nM) occurred 15 min prior to assessment of Caᵢ²⁺ release evoked by increasing concentrations of 5-HT (vehicle, 10⁻¹¹ to 10⁻⁶ M) in h5-HT_{2A}R-CHO cells. Data are presented as maximal 5-HT-induced Caᵢ²⁺ release (Eₘₐₓ). *p <0.05. Comparisons between means for Eₘₐₓ were conducted with an unpaired t-test with Welch's correction (GraphPad Prism). All statistical analyses were conducted with an experiment-wise error rate of α = 0.05.

Regarding FIGS. 11A and 11B, concentration-response curves are shown for compounds 9, 11, 13, 19, and 28 for 5-HT-induced Caᵢ²⁺ release in live (A) h5-HT_{2C}R-CHO cells or (B) h5-HT_{2A}R-CHO cells. Representative curves demonstrate test compounds against concentration-response curve for 5-HT in the absence (black circles) and in the presence (closed triangles); vehicle (open circle); vehicle in the presence of test compound (open triangle). The maximum 5-HT-induced Caᵢ²⁺ release in the absence of the test compounds was set as 100% and the Eₘₐₓ of the test compounds are listed in Table 1 and Table 2.

TABLE 3
Effects of representative 5-HT_{2C}R PAMs and dual 5-HT_{2C}R/5-HT_{2A}R
PAMs (1 nM) on 5-HT-induced Caᵢ²⁺ release in h5-HT_{2B}R-CHO cells

| | | Eₘₐₓ RFU |
|---|---|---|
| Compound | PH | (% 5-HT)^{a} |
| 8 | 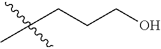 [SMILES: *CCCO] | 95.6 ± 4.6 p = 0.3903 |
| 9 | 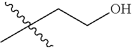 [SMILES: *CCO] | 106.1 ± 7.5 p = 0.4464 |
| 11 | 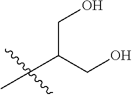 [SMILES: *C(CO)CO] | 97.17 ± 9.2 p = 0.7675 |
| 12 | 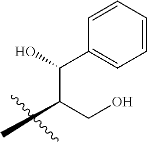 | 92.4 ± 3.2 p = 0.0735 |
| 13 | 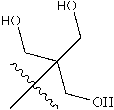 [SMILES: *C(CO)(CO)CO] | 98.41 ± 4.6 p = 0.7429 |
| 25 | 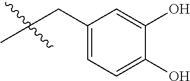 [SMILES: *Cc1ccc(O)c(O)c1] | 100.6 ± 4.4 p = 0.9004 |

^{a}Addition of the synthetic compound (1 nM) occurred 15 min prior to assessment of Caᵢ²⁺ release evoked by increasing concentrations of 5-HT (vehicle, 10⁻¹¹ to 10⁻⁶ M) in h5-HT_{2B}R-CHO cells. Data are presented as maximal 5-HT-induced Caᵢ²⁺ release (Eₘₐₓ). *p <0.05. Comparisons between means for Eₘₐₓ were conducted with an unpaired t-test with Welch's correction (GraphPad Prism). All statistical analyses were conducted with an experiment-wise error rate of α = 0.05.

### Example 4: In Vitro Radioligand Binding Displacement Study

Considering the dual in vitro activity at the 5-HT_{2C}R and 5-HT_{2A}R, compounds 11 and 13 were selected as representative tool compounds out of the active analogs for further pharmacological evaluation. To explore the off-target profile of the two compounds, the National Institute of Mental Health (NIMH) Psychoactive Drug Screening Program (PDSP) were utilized to assess in a broad-panel of GPCRs and monoamine transporters (Table 4). Generally, compounds 11 and 13 exhibited no off-target effects for most of the receptors and monoamine transporters evaluated, except that 11 exhibited a 78.5% average inhibition at 5-HT_{2C}R (Kᵢ=0.46 μM). Compound 13 has no obvious orthosteric displacement for 5-HT_{2A}R or 5-HT_{2C}R, suggesting which is an important feature for selectivity of a 5-HT_{2C}R PAM. Both compounds 11 and 13 did not displace binding to the human ether-a-go-go-related gene (hERG) potassium channel, suggesting a low risk of cardiac adverse events. Compound 13 featured micromolar displacement at H3 receptor (Kᵢ=5.3 μM) and σ₂ receptor (Kᵢ=3.6 μM).

### Example 5: In Vivo Pharmacokinetics and Brain Penetration Analyses

Male Sprague-Dawley rats (n=3/treatment group; Beijing Vital River Laboratory, Animal Technology Co., Ltd., Beijing, China) weighing 200-250 g at the beginning of the experiment were housed three per cage in a pathogen-free, temperature-controlled (20˜26° C.), and humidity-controlled (40˜70%) environment with a 12 h light-dark cycle and ad libitum access to food and filtered water. Rats were randomly assigned to treatment groups. Vehicle [10% dimethyl sulfoxide (DMSO) and 90% 2-hydroxypropyl-β-cyclodextrin (HP-β-CD); Cyclodextrin Technologies Development, Inc., High Springs, FL, USA] or compound 13 dissolved in vehicle was administered to rats ip at 10 mg/kg or po at 20 mg/kg. Blood samples (300 μL) were collected from the jugular vein before dosing and at 0.08, 0.25, 0.5, 1.0, 2.0, 4.0, 8.0, and 24 h postdosing for ip administration and 0.25, 0.5, 1.0, 2.0, 4.0, 8.0, and 24 h postdosing for po administration. The blood samples were placed in heparinized tubes and centrifuged at 6000 rpm for 5 min at 4° C. Brain samples were collected at 0.25 and 1 h postdosing. All samples were stored at −20° C. The concentration of 13 in each sample was analyzed by Sundia MediTech Co., Ltd. The PK parameters of compound 13 were calculated according to a noncompartmental model using WinNonlin 8.1 (Pharsight Corporation, ver 5.3, Mountain View, CA, USA). The peak concentration (Cₘₐₓ) and time of peak concentration (Tₘₐₓ) were directly obtained from the plasma concentration-time profile. The elimination rate constant (λ) was obtained by the least-squares fitted terminal log-linear portion of the slope of the plasma concentration-time profile. The elimination half-life (t_{1/2}) was evaluated according to 0.693/λ. The area under the plasma concentration-time curve from 0 to time t (AUC₀₋ₜ) was evaluated using the linear trapezoidal rule and further extrapolated to infinity (AUC_{0-inf}) following equation: AUC_{0-inf}=AUC₀₋ₜ+Clast/λ. The PK parameters and brain concentrations are presented as mean±SEM.

### Example 6: CNS Multiparameter Optimization

A central nervous system (CNS) multiparameter optimization (MPO) value for compound 13 was calculated. The CNS MPO was adopted to increase the odds of prospectively designing CNS-targeted molecules that achieve CNS exposure. The calculated score for compound 13 is 3.3 out of a collective score range from 0 to 6. Of note, for this series of molecules, the predictive nature is inherently limited by a lower number of structurally comparable molecules in the training data set. It has been suggested that a higher MPO value is desirable for the CNS drugs.

TABLE 4
Displacement of radioligand binding by compound 13
in a broad panel of receptors and transporters.

| | | | Kᵢ |
|---|---|---|---|
| Receptor/Transporter | Radioligand | % inhibition^{a} | (μM)^{b} |
| 5-HT_{1A} | [³H]-8-OH-DPAT | 13.99 | NT |
| 5-HT_{1B} | [³H]-GR125743 | −5.9 | NT |
| 5-HT_{1D} | [³H]-GR125743 | −6.35 | NT |
| 5-HT_{1E} | [³H]-5-HT | 6.48 | NT |
| 5-HT_{2A} | [³H]-Ketanserin | 2.03 | NT |
| 5-HT_{2B} | [³H]-LSD | 33.25 | NT |
| 5-HT_{2C} | [³H]-Mesulergine | 44.64 | NT |
| 5-HT₃ | [³H]-LY278584 | 5.63 | NT |
| 5-HT_{5A} | [³H]-LSD | −7.85 | NT |
| 5-HT₆ | [³H]-LSD | −8.61 | NT |
| 5-HT₇ | [³H]-LSD | −8.95 | NT |
| D₁ | [³H]-SCH23390 | 22.64 | NT |
| D₂ | [³H]-N-Methylspiperone | 2.44 | NT |
| D₃ | [³H]-N-Methylspiperone | −6.88 | NT |
| D₄ | [³H]-N-Methylspiperone | 19.02 | NT |
| D₅ | [³H]-SCH23390 | 56.32 | >10 _{Avg.} |
| DAT | [³H]-WIN35428 | 4.01 | NT |
| SERT | [³H]-Citalopram | −20.8 | NT |
| NET | [³H]-Nisoxetine | −0.8 | NT |
| α _{2A} | [³H]-Rauwolscine | 28.38 | NT |
| α _{2B} | [³H]-Rauwolscine | 14.82 | NT |
| α _{2C} | [³H]-Rauwolscine | 20.26 | NT |
| HERG | [³H]Dofetilide | 1.6 | NT |

^{a}The binding replacement test was conducted at 10 μM of compound 13. A binding inhibition result >50% was considered reliable replacement of target receptor radioligand by the test compound.
^{b}The Kᵢ value was calculated via a non-linear regression analysis of radioligand competition isotherms for ligand binding inhibition >50%.
NT = not tested, Avg. = average Kᵢ from repeated experiments.

### Example 7: Toxicity Profile of Compound 13

In silico toxicity predictions were conducted to evaluate compound 13 in various toxicity endpoints such as acute toxicity, hepatotoxicity, cytotoxicity, carcinogenicity, mutagenicity, immunotoxicity, adverse outcomes (Tox21) pathways and toxicity targets (Table 5). The results suggest that compound 13 exhibits a profile consistent with low expectation of adverse drug reactions or toxic effects and was ranked in a non-toxic class VI (LD₅₀>5,000 mg/kg). A study of CYP450 inhibition was then carried out in human liver microsomes to assess the inhibitory potential of compound 13 (10 μM) against CYP450 isoforms (Table 5). Compound 13 displayed <20% inhibition of CYP3A4, CYP1A2, CYP2C8, CYP2C19, CYP2D6, and CYP2C9 and >50% inhibition of CYP2B6.

TABLE 5
Toxicity profile and human liver microsomes P450 inhibition profile for 13^{a}
In silico toxicity profile

| Predicted LD₅₀ | 10000 mg/kg |
|---|---|
| Toxicity class^{b} | 6 (low) |
| Prediction accuracy | 70.97% |

| | Prediction | | Prediction |
|---|---|---|---|
| Target^{c} | (Probability) | Target^{c} | (Probability) |
| Hepatotoxicity | Inactive (0.84) | AR-LBD | Inactive (0.98) |
| Carcinogenicity | Inactive (0.59) | Aromatase | Inactive (0.99) |
| Immunotoxicity | Inactive (0.96) | PPAR-Gamma | Inactive (0.98) |
| Mutagenicity | Inactive (0.89) | Nrf2/ARE | Inactive (0.94) |
| Cytotoxicity | Inactive (0.82) | HSE | Inactive (0.94) |
| Aryl hydrocarbon | Inactive (0.98) | MMP | Inactive (0.95) |
| receptor | | | |
| Estrogen receptor α | Inactive (0.89) | Phosphoprotein p53 | Inactive (0.96) |
| ER-LBD | Inactive (0.97) | ATAD5 | Inactive (0.99) |
| Androgen receptor | Inactive (0.98) | | |

| | Human liver microsomes P450 inhibition profile | | | | | | | |
|---|---|---|---|---|---|---|---|---|
| P450 | CYP3A4^{d} | CYP3A4^{e} | CYP1A2 | CYP2B6 | CYP2C8 | CYP2C9 | CYP2C19 | CYP2D6 |
| Inhibitory % | 18.72 | 11.11 | 3.4 | 53.31 | 11.87 | 21.2 | 18.34 | 10.13 |

^{a}For information on in silico toxicity predicition, see http://tox.charite.de/protox II/). Cytochrome P450 enzymatic inhibition assays for compound 13 performed at 10 μM and represented as percent inhibition.
^{b}Toxicity class ranks from 1 to 6, 1 = high, 6 = low.
^{c}ER-LBD = Estrogen Receptor Ligand Binding Domain; AR-LBD = Androgen Receptor Ligand Binding Domain; PPAR-Gamma = Peroxisome Proliferator Activated Receptor Gamma; Nrf2/ARE = Nuclear factor (erythroid-derived 2)-like 2/antioxidant responsive element; HSE = Heat shock factor response element; MMP = Mitochondrial Membrane Potential; ATAD5 = ATPase family AAA domain-containing protein 5.
^{d}CYP3A4 (midazolam).
^{e}CYP3A4 (testosterone).

### Example 8: Pharmacokinetics (PK) of Compound 13

An in vitro membrane permeability evaluation of compound 13 was carried out in hMDRI-MDCKII cells to investigate potential CNS permeability and drug efflux. As summarized in, compound 13 demonstrated a moderate permeability and a low efflux ratio of 0.6 and 0.4, respectively, in the absence or presence of a P-glycoprotein (Pgp) inhibitor. Compound 13 displayed a kinetic solubility in PBS buffer of 48.55 μg/mL. The rate of disappearance of 13 following incubation with rat or human liver microsomes was monitored to determine the in vitro intrinsic clearance; 13 showed a higher clearance rate than prior 5-HT_{2C}R PAMs.

In vivo PK evaluation of compound 13 in male Sprague-Dawley rats after a single dose of 10 mg/kg by intraperitoneal (ip) or 20 mg/kg by oral (po) administration was performed to assess drug-like properties and in vivo probe potential of 13. As summarized in Table 7, 10 mg/kg of 13 administered ip (t_{1/2}=2.41 ±1.73 h) or 20 mg/kg of 13 administered po (2.14±0.18 h) resulted in a similar plasma exposure (AUC_{0-inf}, ip: 1,885±232 ng·h·mL⁻¹; po: 615±94 ng·h·mL⁻¹) to 5-HT_{2C}R PAM 2 (AUC_{0-inf}; intravenous: 939±108 ng·h·mL⁻¹; po: 737±56 ng·h·mL⁻¹), although slightly inferior to 5-HT_{2C}R PAM 3.^{42, 44} Compound 13 exhibited brain/plasma (b/p) ratios of 0.589 (15 min) and 2.05 (1 h) after intraperitoneal administration, significantly higher than 0.3, which has been reported as a cutoff to classify CNS drugs.

TABLE 6
In vitro PK data for compound 13

| | |
|---|---|
| MDCK-MDR1 permeability | Without Pgp inhibitor: |
| | Papp_{A→B} = 5.86 × 10⁻⁶ cm/s |
| | Papp_{B→A} = 3.23 × 10⁻⁶ cm/s |
| | Efflux ratio: 0.6 |
| | With Pgp inhibitor: |
| | Papp_{A→B} = 10.64 × 10⁻⁶ cm/s |
| | Papp_{B→A} = 4.77 × 10⁻⁶ cm/s |
| | Efflux ratio: 0.4 |
| Kinetic solubility | 48.55 μg/mL |
| Liver microsomal clearance | CL_{R} = 174.66 μL/min/mg |
| | CL_{H} = 148.85 μL/min/mg |

TABLE 7
In vivo PK and brain penetrability for Compound 13^{a}
In vivo PK profile

| Dose | T_{1/2} | Tₘₐₓ | Cₘₐₓ | AUC_{0-inf} |
|---|---|---|---|---|
| (mg/kg) | (h) | (h) | (ng/mL) | (ng · h · mL⁻¹) |
| 10, ip | 2.41 ± 1.73 | 0.5 | 878 ± 236 | 1885 ± 232 |
| 20, po | 2.14 ± 0.18 | 0.667 ± 0.289 | 181 ± 36 | 615 ± 94 |

Brain penetration analysis

| Dose | Time | Brain conc | Plasma conc | Brain/plasma |
|---|---|---|---|---|
| (mg/kg) | (h) | (ng/g) | (ng/mL) | ratio |
| 10, ip | 0.25 | 378 ± 58.8 | 642 ± 133 | 0.589 |
| 10, ip | 1.0 | 1120 ± 65 | 547 ± 18.3 | 2.05 |

^{a}T_{1/2}, half-life; Tₘₐₓ, time of maximum concentration; Cₘₐₓ, maximum concentration; AUC_{0-inf}, area under the plasma concentration-time curve; time, hours after dose for brain collection; brain conc, averaged concentration of 13 in tissue sample. Experiments were studied in biological triplicates, and data values are shown as the mean ± SEM (±standard error of the mean) from male Sprague-Dawley rats. Vehicle, 10% dimethyl sulfoxide (DMSO): 90% 2-hydroxypropyl-β-cyclodextrin (HP-β-CD). analog

### 4.0 Exemplary Embodiments

The present invention includes the following nonlimiting exemplary embodiments:

1. In some embodiments, the invention encompasses a compound according to Formula (I)

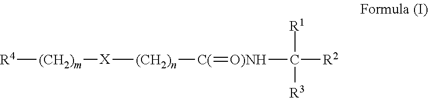

or a pharmaceutically acceptable salt thereof,
wherein:
R¹, R² and R³ are independently chosen from H, substituted or unsubstituted aryl, heteroaryl, arylalkyl, heteroaryl alkyl, carbonyl, C1 to C6 alkyl, C1 to C6 heteroalkyl, C1 to C6 alkoxy, C1 to C6 hydroxyalkyl, C1 to C6 ester, and heterocyclyl;
R⁴ is chosen from H, substituted or unsubstituted aryl, substituted or unsubstituted heteroaryl, arylalkyl and heteroaryl alkyl;
X is selected from the group consisting of:
—CH₂CH₂—, —CH═CH—,

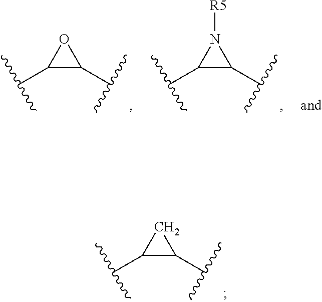

R⁵ is chosen from H, C1-C6 alkyl, and arylalkyl;
m is 0-20; and
n is 1-20.

2. In some embodiments, the invention encompasses a compound of Formula I wherein R¹ is H.

3. In some embodiments, the invention encompasses a compound of Formula I wherein R¹ and R² are H.

4. In some embodiments, the invention encompasses a compound of Formula I wherein X is —CH═CH—.

5. In some embodiments, the invention encompasses a compound of Formula I wherein X is —CH═CH— and wherein the —CH═CH— group is the cis-isomer.

6. In some embodiments, the invention encompasses a compound of Formula I wherein X is —CH═CH—, wherein the —CH═CH— group is the cis-isomer, and wherein m is 8, n is 7, and R⁴ is H.

7. In some embodiments, the invention encompasses a compound of Formula I wherein X is

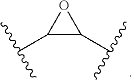 [SMILES: *C1OC1*]

8. In some embodiments, the invention encompasses a compound of Formula I wherein X is

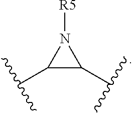

9. In some embodiments, the invention encompasses a compound of Formula I wherein X is

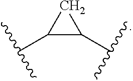 [SMILES: *C1CC1*]

10. In some embodiments, the invention encompasses a compound of Formula I wherein X is —CH₂CH₂—.

11. In some embodiments, the invention encompasses a compound of Formula I wherein R⁴ is chosen from H, substituted or unsubstituted aryl, substituted or unsubstituted heteroaryl, arylalkyl and heteroaryl alkyl.

12. In some embodiments, the invention encompasses a compound of Formula I wherein R⁴ is H.

13. In some embodiments, the invention encompasses a compound of Formula I, or a pharmaceutically acceptable salt thereof, wherein the compound is chosen from any of:

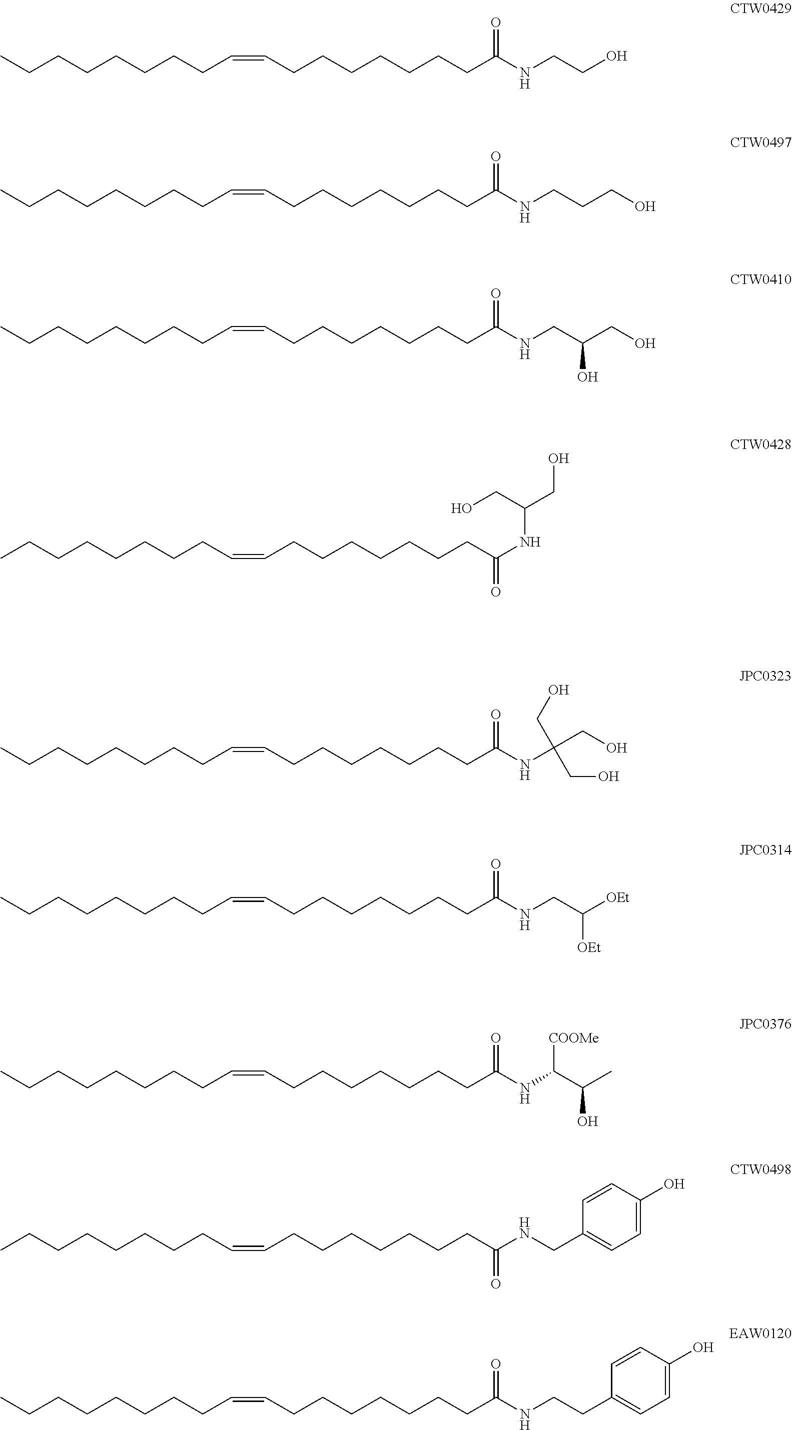

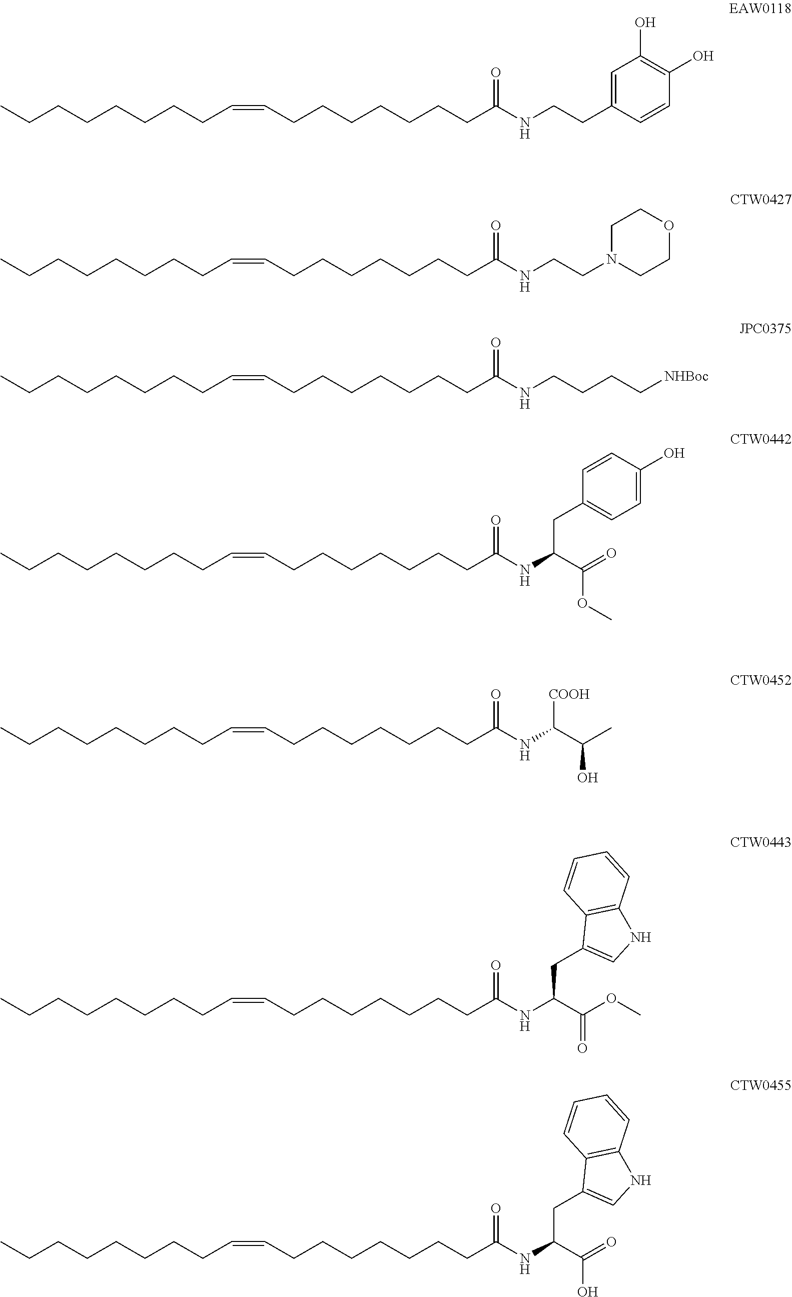

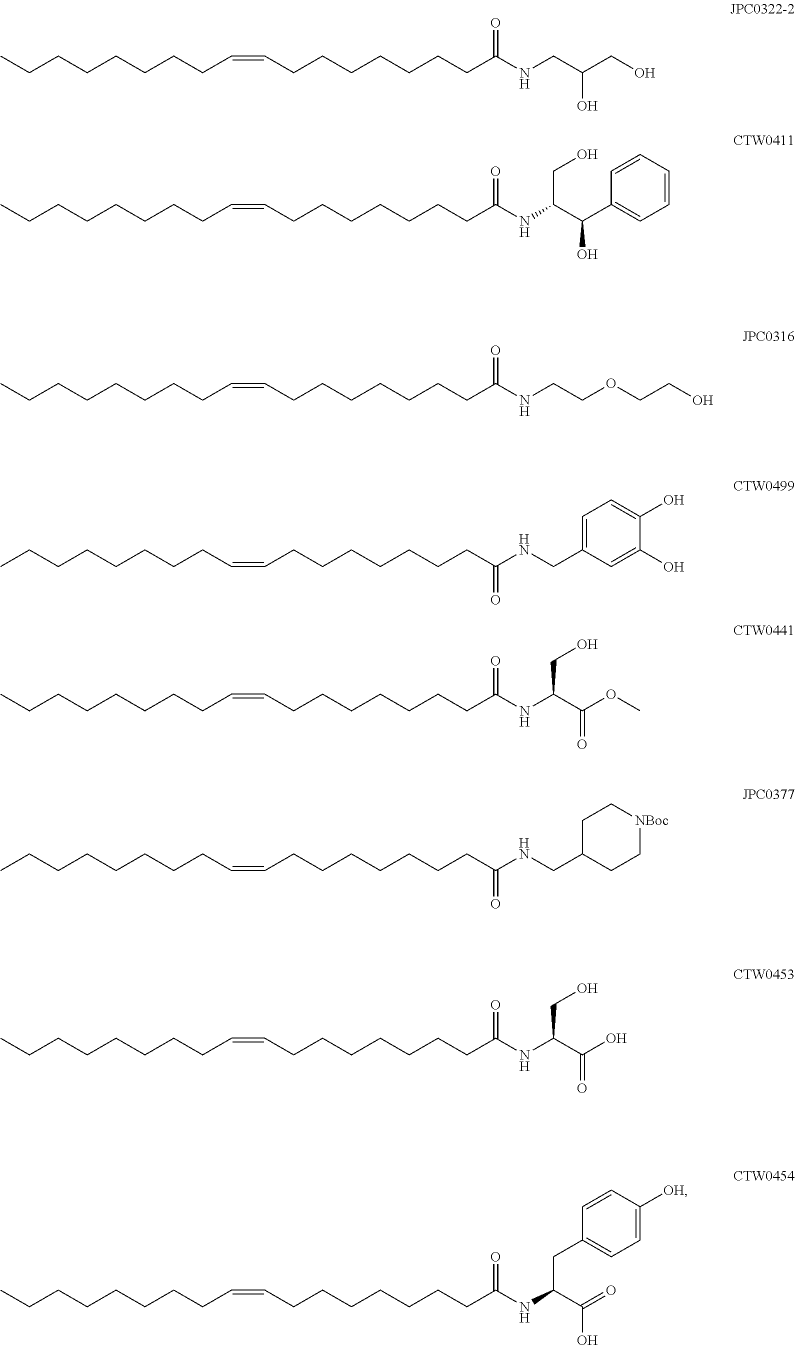

and combinations thereof.

14. In some embodiments, the invention encompasses a compound of Formula Ia, wherein the compound is:
CH₃—(CH₂)₇-((cis)-CH═CH—)—(CH₂)₇—C(═O)NH—C(CH₂OH)₃ Formula Ia or a pharmaceutically acceptable salt thereof.

15. In some embodiments, the invention encompasses a compound of Formula I, or a pharmaceutically acceptable salt thereof, wherein the compound is chosen from any of:

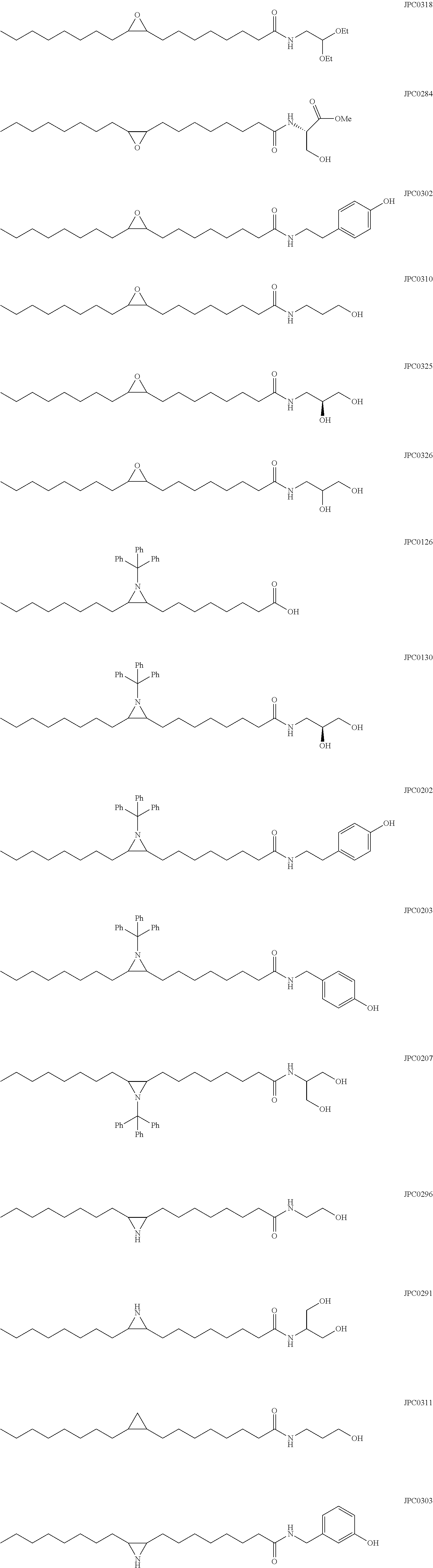

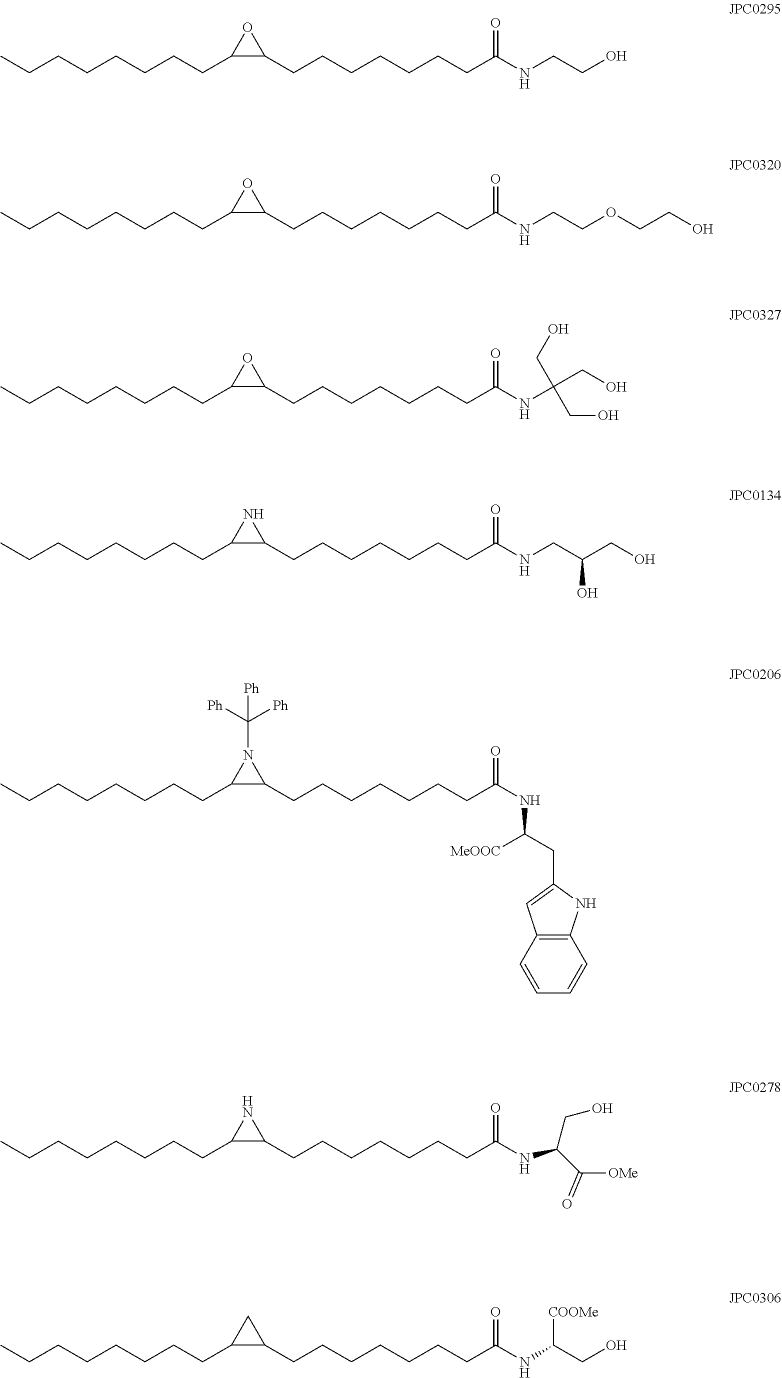

16. In some embodiments, the invention encompasses a compound of Formula I, or a pharmaceutically acceptable salt thereof, wherein the compound is chosen from any of:

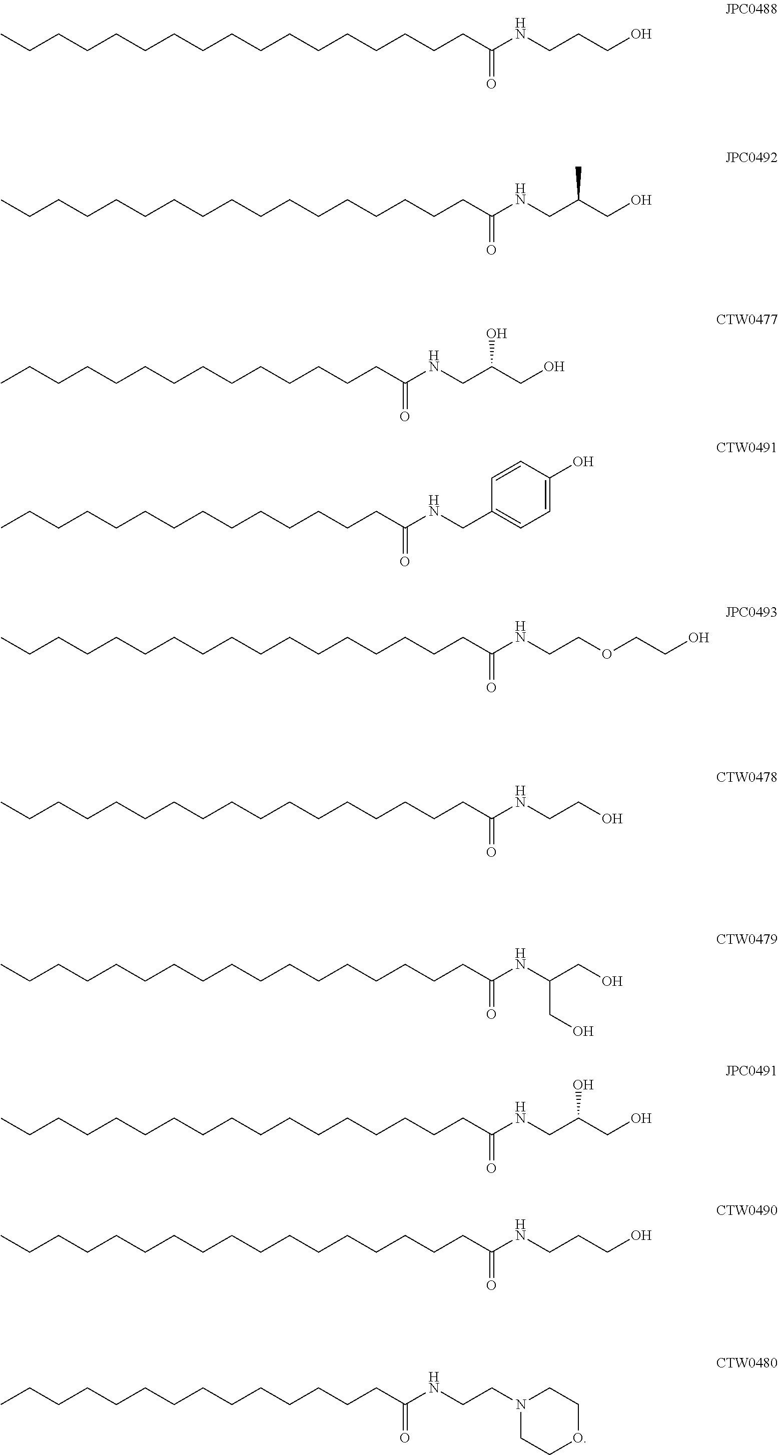

17. In some embodiments, the invention encompasses a compound of Formula I, or a pharmaceutically acceptable salt thereof, wherein the compound is chosen from any of:

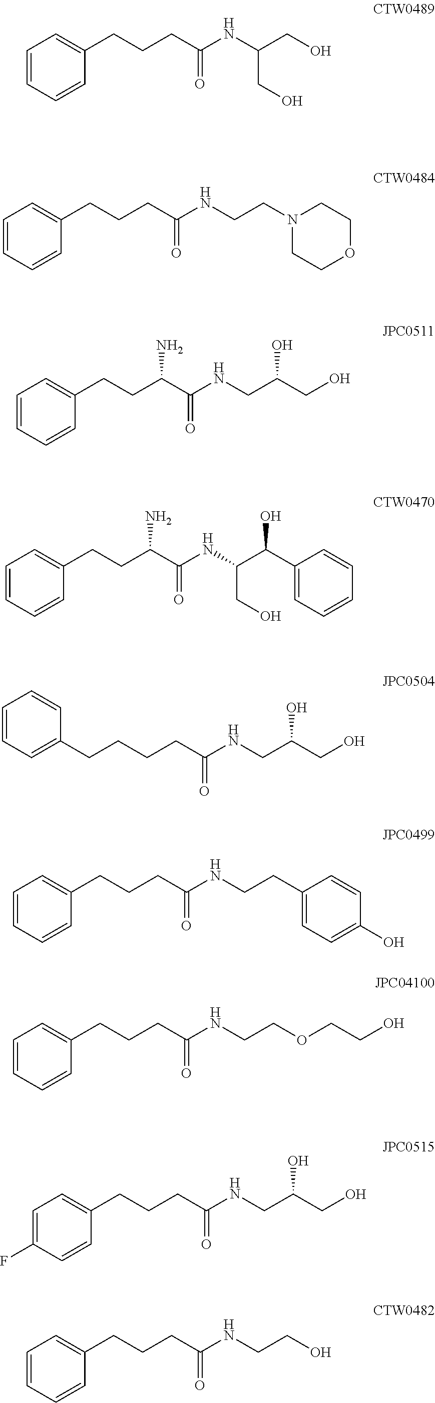

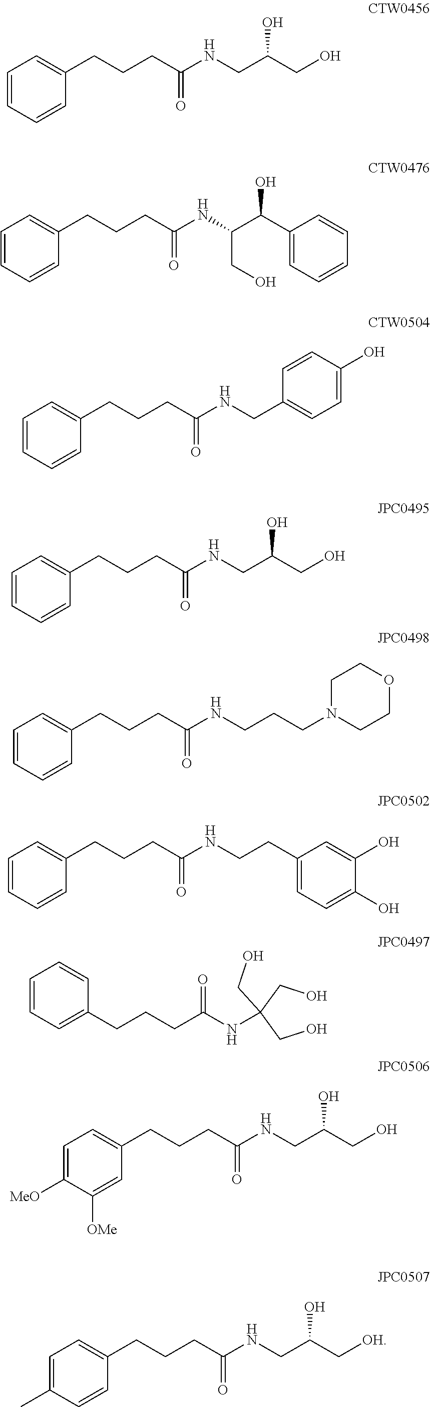

18. In some embodiments, the invention encompasses a compound chosen from any of those listed in FIG. 10A, 10B, 11A, or 11B, or a pharmaceutically acceptable salt thereof.

19. In some embodiments, the invention encompasses a compound according to Formula (Ia)

R⁴—(CH₂)ₘ—X—(CH₂)ₙ—C(═O)NH—(PH) Formula (Ia)
or a pharmaceutically acceptable salt thereof,
wherein:
R⁴ is H, substituted or unsubstituted aryl, substituted or unsubstituted heteroaryl, arylalkyl or heteroaryl alkyl;
X is selected from the group consisting of:
—CH₂CH₂—, —CH═CH—,

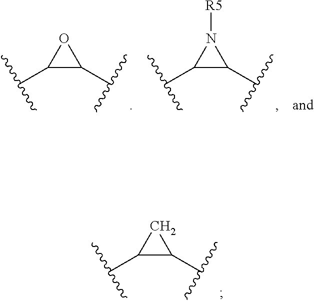

R⁵ is selected from H, C1-C6 alkyl, arylalkyl;
m is 0-20; and
n is 1-20;
and (PH) is chosen from any of the following groups numbered 7-30.

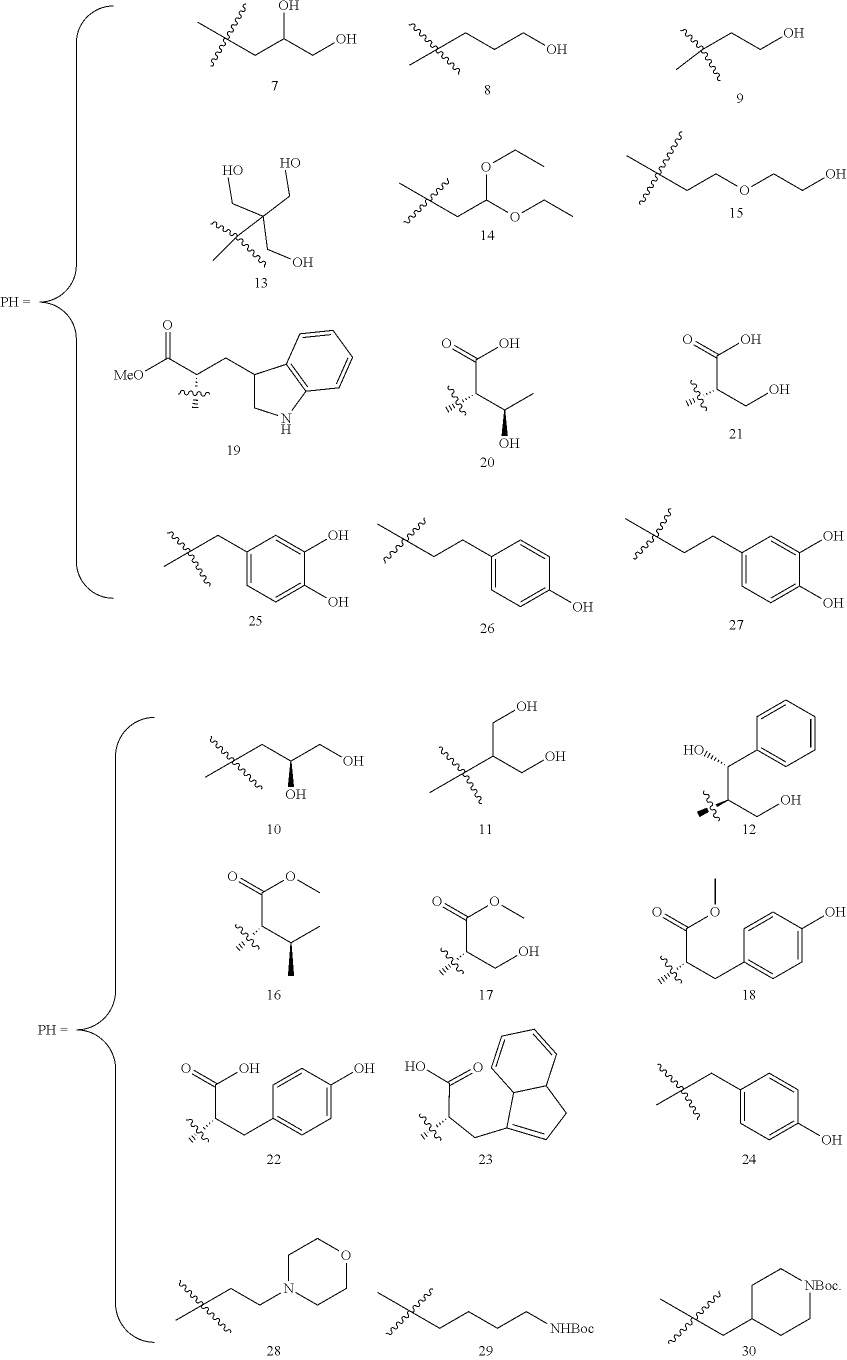

20. In some embodiments, the invention encompasses a compound of Formula Ia wherein (PH) is —C(CH₂OH)₃.

21. In some embodiments, the invention encompasses a compound of Formula Ia wherein X is (—CH═CH—); and
wherein (PH) is —C(CH₂OH)₃.

22. In some embodiments, the invention encompasses a compound of Formula Ia wherein X is (—CH═CH—);
m is 8; n is 7; R⁴ is H; and
wherein (PH) is —C(CH₂OH)₃.

23. In some embodiments, the invention encompasses compound 13, of the formula:

CH₃—(CH₂)₇-((cis)-CH═CH—)—(CH₂)₇—C(═)NH—C(CH₂OH)₃ Compound 13
or a pharmaceutically acceptable salt thereof.

24. In some embodiments, the invention encompasses a compound of Formula I, or a pharmaceutically acceptable salt thereof, wherein the compound is chosen from any of:

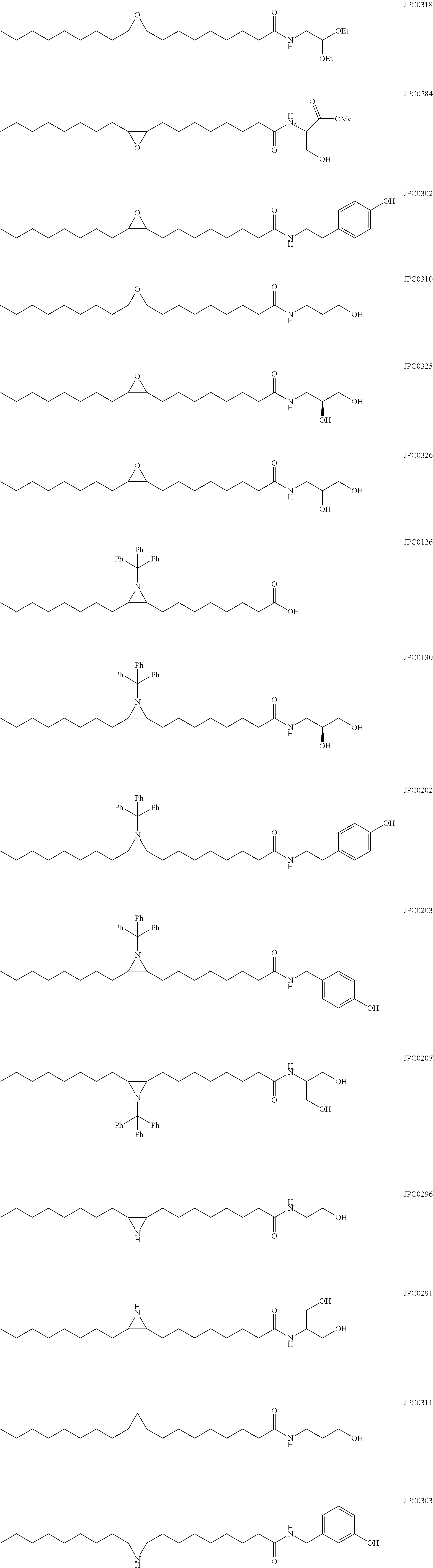

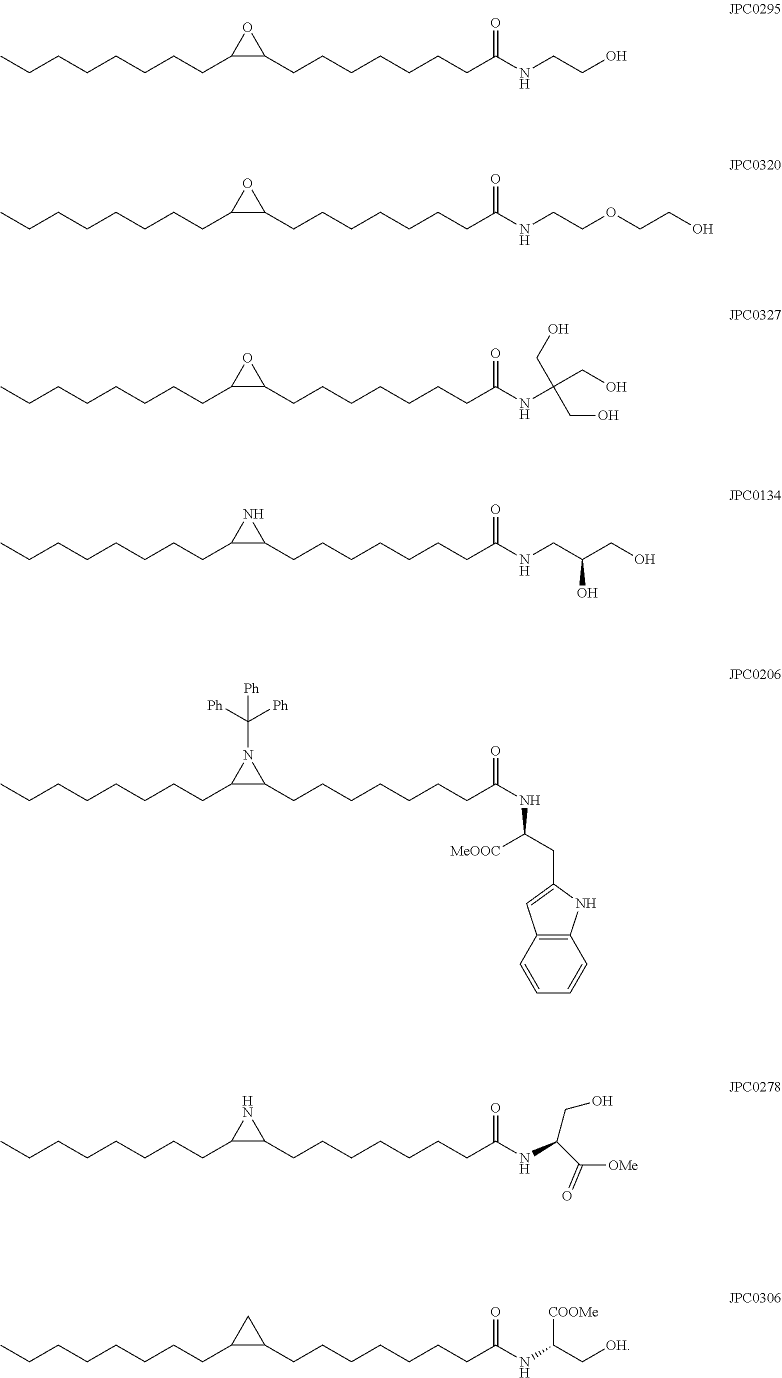

25. In some embodiments, the invention encompasses a compound of Formula I, or a pharmaceutically acceptable salt thereof, wherein the compound is chosen from any of:

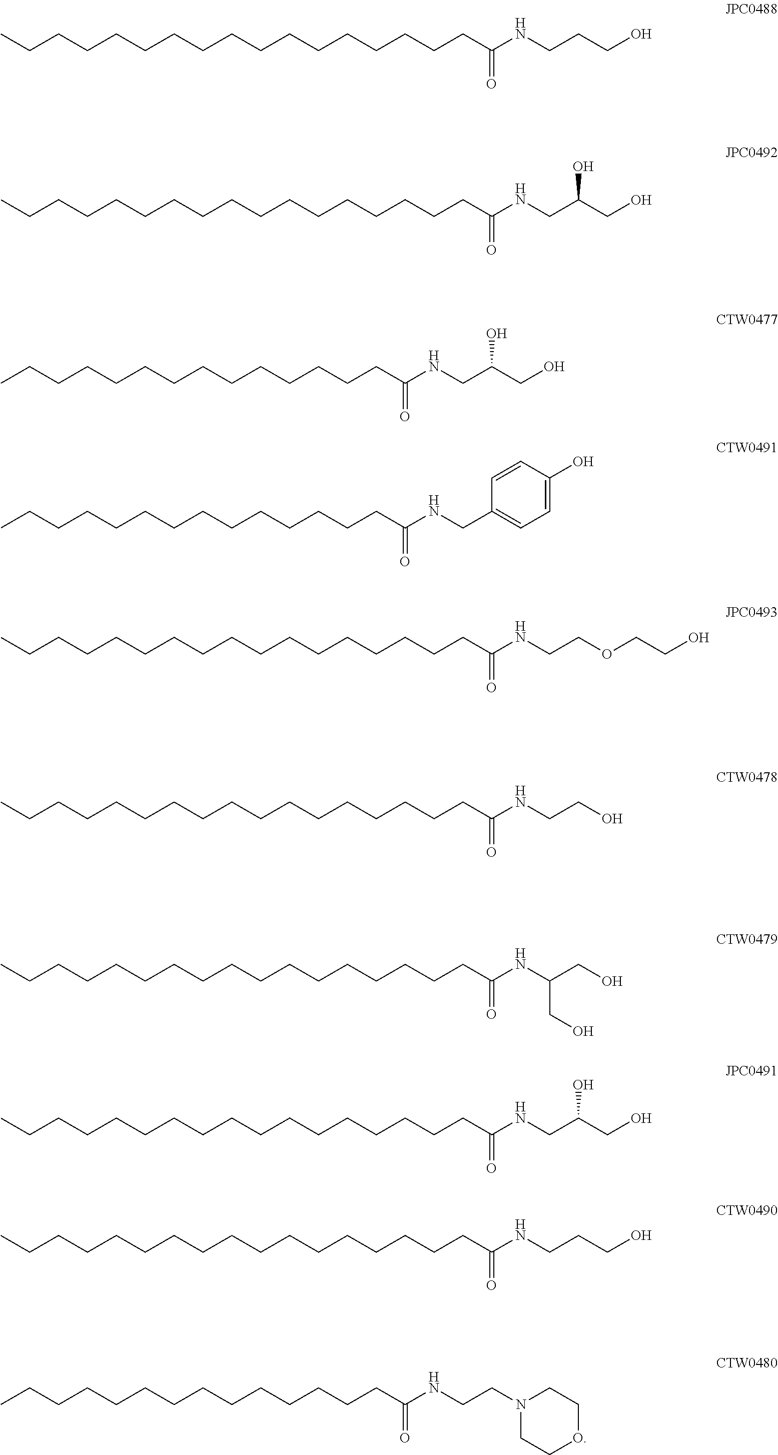

26. In some embodiments, the invention encompasses a compound of Formula I, or a pharmaceutically acceptable salt thereof, wherein the compound is chosen from any of:

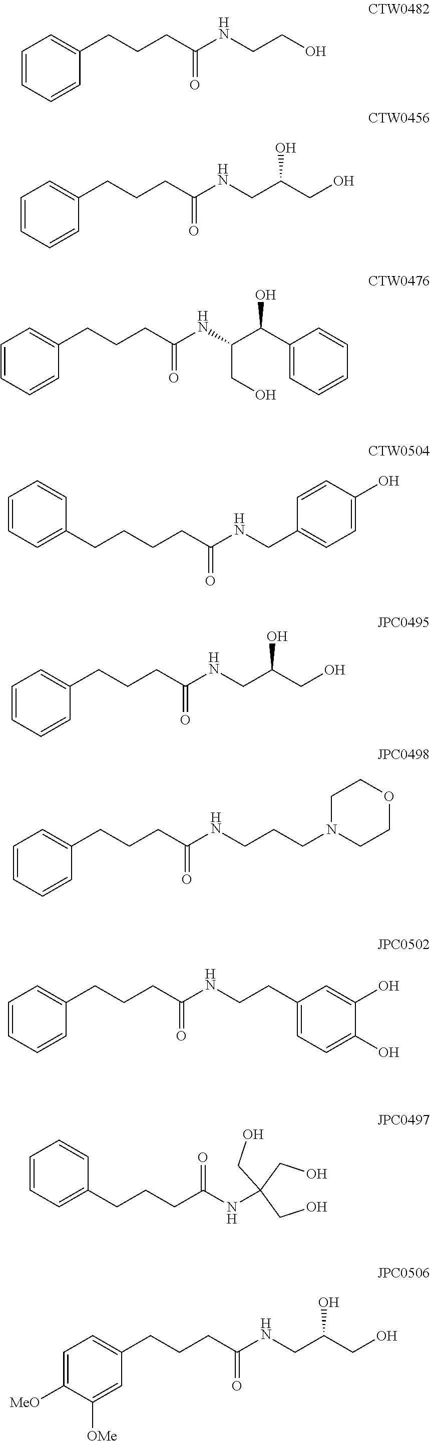

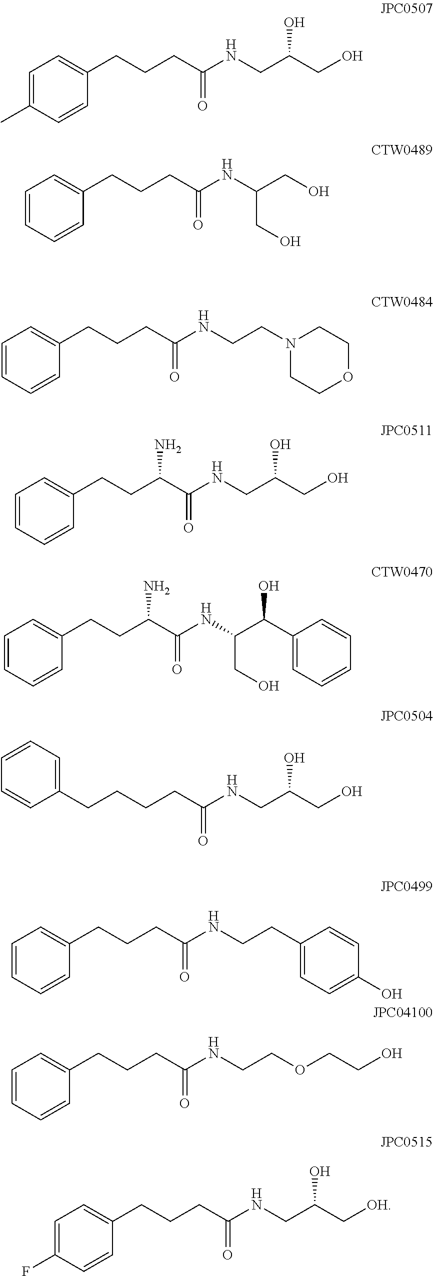

27. In some embodiments, the invention encompasses a compound according to Formula (II):

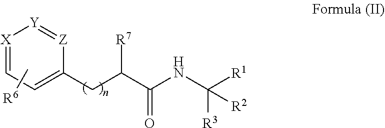

or a pharmaceutically acceptable salt thereof,
wherein R¹, R², R³ are independently chosen from H, substituted or unsubstituted aryl, heteroaryl, arylalkyl, heteroaryl alkyl, carbonyl, C1 to C6 alkyl, C1 to C6 heteroalkyl, C1 to C6 alkoxy, C1 to C6 hydroxyalkyl, and C1 to C6 ester;
R⁶ is chosen from H, OH, NO₂, amino, CF₃, halogen, alkyl, or alkoxy;
R⁷ is chosen from H and —NR⁸R⁹;
R⁸, R⁹ are independently chosen from H, alkyl, aryl, arylalkyl, cycloalkyl, alkoxy, heteroalkyl, or
R⁸, R⁹ taken together with other atoms to form a 5- or 6-membered ring;
X, Y, Z are independently CH and N; and
n is 1 to 7.

28. In some embodiments, the invention encompasses a compound according to Formula (II), or a pharmaceutically acceptable salt thereof, wherein R⁷ is —NR⁸R⁹.

29. In some embodiments, the invention encompasses a compound according to Formula (II), or a pharmaceutically acceptable salt thereof, wherein R⁷ is —NR⁸R⁹ and wherein the structure of the compound is selected from Formula (II)-(R) (also referred to as Formula (IIa)) and Formula (II)-(S) (also referred to as Formula (IIb)):

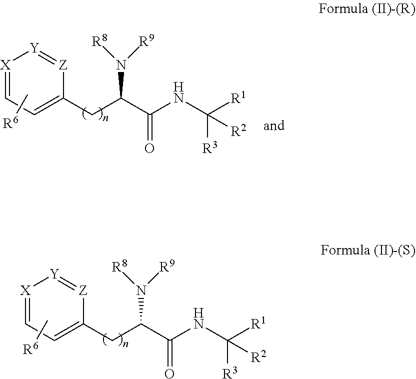

wherein the Formula (II)-(R) and Formula (II)-(S) designations refer to the respective R and S configurations at the chiral center carbon bonded to —NR⁸R⁹.

30. A method of treating a disease or condition, said method comprising administering to a patient a therapeutically effective amount of one or more compounds chosen from any of Formulas I, Ia, II, IIa, and IIb, and any combination of thereof, (or a pharmaceutically acceptable salt thereof).

31. In some embodiments, the invention encompasses said method of treating a disease or condition, according to embodiment 30, wherein treating the disease or condition involves the modulation of 5-hydroxytryptamine 2A receptor and/or 5-hydroxytryptamine 2C receptor.

32. In some embodiments, the invention encompasses said method of treating a disease or condition, according to embodiment 30, wherein the disease or condition may be treated by modulating 5-hydroxytryptamine 2A receptor and/or 5-hydroxytryptamine 2C receptor.

33. In some embodiments, the invention encompasses said method of treating a disease or condition, according to embodiment 30, wherein the compound according the invention modulates 5-hydroxytryptamine 2A receptor and/or 5-hydroxytryptamine 2C receptor.

34. In some embodiments, the invention encompasses said method of treating a disease or condition, according to embodiment 30, wherein the disease or condition responsive is a substance use disorder, a psychiatric or neurological disorder, obesity, a mood disorder or a seizure disorder.

35. In some embodiments, the invention encompasses said method of treating a disease or condition, according to embodiment 30, wherein the compound is administered intravenously.

36. In some embodiments, the invention encompasses said method of treating a disease or condition, according to embodiment 30, wherein said patient has a diagnosis of a substance use disorder, obesity, a mood disorder or a seizure disorder.

### REFERENCES

Howell, L. L.; Cunningham, K. A. Serotonin 5-ht2 receptor interactions with dopamine function: Implications for therapeutics in cocaine use disorder. Pharmacol. Rev. 2015, 67, 176-197.
Anastasio, N. C.; Liu, S.; Maili, L.; Swinford, S. E.; Lane, S. D.; Fox, R. G.; Hamon, S. C.; Nielsen, D. A.; Cunningham, K. A.; Moeller, F. G. Variation within the serotonin (5-ht) 5-ht₂c receptor system aligns with vulnerability to cocaine cue reactivity. Transl. Psychiatry 2014, 4, e369.
Anastasio, N. C.; Stutz, S. J.; Fink, L. H.; Swinford-Jackson, S. E.; Sears, R. M.; DiLeone, R. J.; Rice, K. C.; Moeller, F. G.; Cunningham, K. A. Serotonin (5-ht) 5-ht2a receptor (5-ht2ar):5-ht2cr imbalance in medial prefrontal cortex associates with motor impulsivity. ACS Chem. Neurosci. 2015, 6, 1248-1258.
Anastasio, N. C.; Stutz, S. J.; Fox, R. G.; Sears, R. M.; Emeson, R. B.; DiLeone, R. J.; O'Neil, R. T.; Fink, L. H.; Li, D.; Green, T. A.; Moeller, F. G.; Cunningham, K. A. Functional status of the serotonin 5-ht2c receptor (5-ht2cr) drives interlocked phenotypes that precipitate relapse-like behaviors in cocaine dependence. Neuropsychopharmacology 2014, 39, 370-382.
Arafat, E. S.; Trimble, J. W.; Andersen, R. N.; Dass, C.; Desiderio, D. M. Identification of fatty acid amides in human plasma. Life Sci. 1989, 45, 1679-1687.
Aznar, S.; Hervig Mel, S. The 5-ht2a serotonin receptor in executive function: Implications for neuropsychiatric and neurodegenerative diseases. Neurosci. Biobehav. Rev. 2016, 64, 63-82.
Banerjee, P.; Eckert, A. O.; Schrey, A. K.; Preissner, R. Protox-ii: A webserver for the prediction of toxicity of chemicals. Nucleic Acids Res. 2018, 46, 257-263.
Barnes, N. M.; Sharp, T. A review of central 5-ht receptors and their function. Neuropharmacology 1999, 38, 1083-1152.
Barrett, F. S.; Doss, M. K.; Sepeda, N. D.; Pekar, J. J.; Griffiths, R. R. Emotions and brain function are altered up to one month after a single high dose of psilocybin. Sci. Rep. 2020, 10, 2214.
Besnard, J.; Ruda, G. F.; Setola, V.; Abecassis, K.; Rodriguiz, R. M.; Huang, X.-P.; Norval, S.; Sassano, M. F.; Shin, A. I.; Webster, L. A.; Simeons, F. R. C.; Stojanovski, L.; Prat, A.; Seidah, N. G.; Constam, D. B.; Bickerton, G. R.; Read, K. D.; Wetsel, W. C.; Gilbert, I. H.; Roth, B. L.; Hopkins, A. L. Automated design of ligands to polypharmacological profiles. Nature 2012, 492, 215-220.
Boger, D. L.; Patterson, J. E.; Jin, Q. Structural requirements for 5-ht2a and 5-ht1a serotonin receptor potentiation by the biologically active lipid oleamide. Proc. Natl. Acad. Sci. U.S.A 1998, 95, 4102-4107.
Cheer, J. F.; Cadogan, A.-K.; Marsden, C. A.; Fone, K. C. F.; Kendall, D. A. Modification of 5-ht2 receptor mediated behaviour in the rat by oleamide and the role of cannabinoid receptors. Neuropharmacology 1999, 38, 533-541.
Conn, P. J.; Christopoulos, A.; Lindsley, C. W. Allosteric modulators of gpcrs: A novel approach for the treatment of cns disorders. Nat. Rev. Drug Discov. 2009, 8, 41-54.
Cunningham, K. A.; Anastasio, N. C. Serotonin at the nexus of impulsivity and cue reactivity in cocaine addiction. Neuropharmacology 2014, 76 460-478.
Cunningham, K. A.; Anastasio, N. C.; Fox, R. G.; Stutz, S. J.; Bubar, M. J.; Swinford,
S. E.; Watson, C. S.; Gilbertson, S. R.; Rice, K. C.; Rosenzweig-Lipson, S.; Moeller, F. G.
Synergism between a serotonin 5-ht2a receptor (5-ht2ar) antagonist and 5-ht2cr agonist suggests Iapharmacotherapeutics for cocaine addiction. ACS Chem. Neurosci. 2013, 4, 110-121.
Cunningham, K. A.; Bubar, M. J.; Anastasio, N. C. The serotonin 5-ht2c receptor in medial prefrontal cortex exerts rheostatic control over the motivational salience of cocaine-associated cues: New observations from preclinical animal research. Neuropsychopharmacology 2010, 35, 2319-2321.
Cunningham, K. A.; Fox, R. G.; Anastasio, N. C.; Bubar, M. J.; Stutz, S. J.; Moeller, F. G.; Gilbertson, S. R.; Rosenzweig-Lipson, S. Selective serotonin 5-ht(2c) receptor activation suppresses the reinforcing efficacy of cocaine and sucrose but differentially affects the incentive-salience value of cocaine- vs. Sucrose-associated cues. Neuropharmacology 2011, 61, 513-523.
Ding, C.; Bremer, N. M.; Smith, T. D.; Seitz, P. K.; Anastasio, N. C.; Cunningham, K.
A.; Zhou, J. Exploration of synthetic approaches and pharmacological evaluation of pnu-69176e and its stereoisomer as 5-ht2c receptor allosteric modulators. ACS Chem. Neurosci. 2012, 3, 538-545.
Dunlop, J.; Sabb, A. L.; Mazandarani, H.; Zhang, J.; Kalgaonker, S.; Shukhina, E.; Sukoff, S.; Vogel, R. L.; Stack, G.; Schechter, L.; Harrison, B. L.; Rosenzweig-Lipson, S. Way-163909 [(7br, 10ar)-1,2,3,4,8,9,10,10a-octahydro-7bh-cyclopenta[b][1,4]diazepino[6,7,1hi]indole], a novel 5-hydroxytryptamine 2c receptor-selective agonist with anorectic activity. J. Pharmacol. Exp. Ther. 2005, 313, 862-869. Fedorova, I.; Hashimoto, A.; Fecik, R. A.; Hedrick, M. P.; Hanus, L. O.; Boger, D. L.; Rice, K. C.; Basile, A. S. Behavioral evidence for the interaction of oleamide with multiple neurotransmitter systems. J. Pharmacol. Exp. Ther. 2001, 299, 332-342.
Fitzgerald, L. W.; Burn, T. C.; Brown, B. S.; Patterson, J. P.; Corjay, M. H.; Valentine, P. A.; Sun, J. H.; Link, J. R.; Abbaszade, I.; Hollis, J. M.; Largent, B. L.; Hartig, P. R.; Hollis, G. F.; Meunier, P. C.; Robichaud, A. J.; Robertson, D. W. Possible role of valvular serotonin 5-ht(2b) receptors in the cardiopathy associated with fenfluramine. Mol. Pharmacol. 2000, 57, 75-81.
Fuentes, J. J.; Fonseca, F.; Elices, M.; Farre, M.; Torrens, M. Therapeutic use of lsd in psychiatry: A systematic review of randomized-controlled clinical trials. Front Psychiatry 2019, 10, 943.
Garcia-Circeles, J.; Decara, J. M.; Vázquez-Villa, H.; Rodriguez, R.; Codesido, E.; Cruces, J.; Brea, J.; Loza, M. I.; Alén, F.; Botta, J.; McCormick, P. J.; Ballesteros, J. A.; Benhamú, B.; Rodriguez de Fonseca, F.; López-Rodríguez, M. L. A positive allosteric modulator of the serotonin 5-ht(2c) receptor for obesity. J. Med. Chem. 2017, 60, 9575-9584.
Garcia-Romeu, A.; Barrett, F. S.; Carbonaro, T. M.; Johnson, M. W.; Griffiths, R. R. Optimal dosing for psilocybin pharmacotherapy: Considering weight-adjusted and fixed dosing approaches. J Psychopharmacol 2021, 269881121991822.
Garrido, A.; Lepailleur, A.; Mignani, S. M.; Dallemagne, P.; Rochais, C. Herg toxicity assessment: Useful guidelines for drug design. Eur. J. Med. Chem. 2020, 195, 112290.
Ge, T.; Zhang, Z.; Lv, J.; Song, Y.; Fan, J.; Liu, W.; Wang, X.; Hall, F. S.; Li, B.; Cui, R. The role of 5-ht(2c) receptor on corticosterone-mediated food intake. J. Biochem. Mol. Toxicol. 2017, 31.
Georgescu, T.; Lyons, D.; Heisler, L. K. Role of serotonin in body weight, insulin secretion and glycaemic control. J. Neuroendocrinol. 2021, 33, e12960.
Heal, D. J.; Gosden, J.; Smith, S. L. Evaluating the abuse potential of psychedelic drugs as part of the safety pharmacology assessment for medical use in humans. Neuropharmacology 2018, 142, 89-115.
Hedlund, P. B.; Carson, M. J.; Sutcliffe, J. G.; Thomas, E. A. Allosteric regulation by oleamide of the binding properties of 5-hydroxytryptamine7 receptors. Biochem. Pharmacol. 1999, 58, 1807-1813.
Hedlund, P. B.; Danielson, P. E.; Thomas, E. A.; Slanina, K.; Carson, M. J.; Sutcliffe, J. G. No hypothermic response to serotonin in 5-ht7 receptor knockout mice. Proc. Natl. Acad. Sci. U.S.A 2003, 100, 1375-1380.
Heng, H. L.; Chee, C. F.; Thy, C. K.; Tee, J. T.; Chin, S. P.; Herr, D. R.; Buckle, M. J. C.; Paterson, I. C.; Doughty, S. W.; Abd Rahman, N.; Chung, L. Y. In vitro functional evaluation of isolaureline, dicentrine and glaucine enantiomers at 5-ht(2) and α(1) receptors. Chem. Biol. Drug Des. 2019, 93, 132-138.
Howell, L. L.; Cunningham, K. A. Serotonin 5-ht2 receptor interactions with dopamine function: Implications for therapeutics in cocaine use disorder. Pharmacol. Rev. 2015, 67, 176-197.
Hynie, S. [5-hydroxytryptamine (serotonin) receptors--nomenclature and classification of types and subtypes]. Cesk. Fysiol. 1995, 44, 135-138.
Im, W. B.; Chio, C. L.; Alberts, G. L.; Dinh, D. M. Positive allosteric modulator of the human 5-ht2c receptor. Mol. Pharmacol. 2003, 64, 78-84.
Inserra, A.; De Gregorio, D.; Gobbi, G. Psychedelics in psychiatry: Neuroplastic, immunomodulatory, and neurotransmitter mechanisms. Pharmacol. Rev. 2021, 73, 202-277.
Johnson, M. W.; Garcia-Romeu, A.; Griffiths, R. R. Long-term follow-up of psilocybin-facilitated smoking cessation. Am. J. Drug Alcohol Abuse 2017, 43, 55-60.
Johnson, M. W.; Hendricks, P. S.; Barrett, F. S.; Griffiths, R. R. Classic psychedelics: An integrative review of epidemiology, therapeutics, mystical experience, and brain network function. Pharmacol. Ther. 2019, 197, 83-102.
Kim, K.; Che, T.; Panova, O.; DiBerto, J. F.; Lyu, J.; Krumm, B. E.; Wacker, D.; Robertson, M. J.; Seven, A. B.; Nichols, D. E.; Shoichet, B. K.; Skiniotis, G.; Roth, B. L. Structure of a hallucinogen-activated gq-coupled 5-ht2a serotonin receptor. Cell 2020, 182, 1574-1588.
Li, L.-B.; Zhang, L.; Sun, Y.-N.; Han, L.-N.; Wu, Z.-H.; Zhang, Q.-J.; Liu, J. Activation of serotonin2a receptors in the medial septum-diagonal band of broca complex enhanced working memory in the hemiparkinsonian rats. Neuropharmacology 2015, 91, 23-33.
López-Giménez, J. F.; González-Maeso, J. Hallucinogens and serotonin 5-ht(2a) receptor-mediated signaling pathways. Curr. Top. Behav. Neurosci. 2018, 36, 45-73.
Lu, C.; Wu, C.; Ghoreishi, D.; Chen, W.; Wang, L.; Damm, W.; Ross, G. A.; Dahlgren, M. K.; Russell, E.; Von Bargen, C. D.; Abel, R.; Friesner, R. A.; Harder, E. D. Opls4: Improving force field accuracy on challenging regimes of chemical space. J. Chem. Theory Comput. 2021, 17, 4291-4300.
Lu, S.; Li, S.; Zhang, J. Harnessing allostery: A novel approach to drug discovery. Med. Res. Rev. 2014, 34, 1242-1285.
Meltzer, H. Y. The role of serotonin in antipsychotic drug action. Neuropsychopharmacology 1999, 21, 106s-115s.
Méndez-Diaz, M.; Amancio-Belmont, O.; Estrada-González, V.; Ruiz-Contreras, A. E.; Prospéro-Garcia, O. Cb1r mediates oleamide's reward while 5ht2cr mediates aversion in the nucleus accumbens shell of rats. Neurosci. Lett. 2019, 706, 189-193.
Millan, M. J.; Marin, P.; Bockaert, J.; Mannoury la Cour, C. Signaling at g-protein-coupled serotonin receptors: Recent advances and future research directions. Trends Pharmacol. Sci. 2008, 29, 454-464.
Mueller, G. P.; Driscoll, W. J. Biosynthesis of oleamide. Vitam. Horm. 2009, 81, 55-78.
Nichols, D. E. Hallucinogens. Pharmacol. Ther. 2004, 101, 131-181.
Nichols, D. E. Psychedelics. Pharmacol. Rev. 2016, 68, 264-355.
Orejarena, M. J.; Lanfumey, L.; Maldonado, R.; Robledo, P. Involvement of 5-ht2a receptors in mdma reinforcement and cue-induced reinstatement of mdma-seeking behaviour. Int. J. Neuropsychopharmacol. 2011, 14, 927-940.
Peng, Y.; McCorvy, J. D.; Harpsee, K.; Lansu, K.; Yuan, S.; Popov, P.; Qu, L.; Pu, M.; Che, T.; Nikolajsen, L. F.; Huang, X. P.; Wu, Y.; Shen, L.; Bjorn-Yoshimoto, W. E.; Ding, K.; Wacker, D.; Han, G. W.; Cheng, J.; Katritch, V.; Jensen, A. A.; Hanson, M. A.; Zhao, S.; Gloriam, D. E.; Roth, B. L.; Stevens, R. C.; Liu, Z. J. 5-ht(2c) receptor structures reveal the structural basis of gpcr polypharmacology. Cell 2018, 172, 719-730.
Pogorelov, V. M.; Rodriguiz, R. M.; Cheng, J.; Huang, M.; Schmerberg, C. M.; Meltzer, H. Y.; Roth, B. L.; Kozikowski, A. P.; Wetsel, W. C. 5-ht(2c) agonists modulate schizophrenia-like behaviors in mice. Neuropsychopharmacology 2017, 42, 2163-2177.
Prospéro-Garcia, O.; Amancio-Belmont, O.; Becerril Melendez, A. L.; Ruiz-Contreras, A. E.; Méndez-Diaz, M. Endocannabinoids and sleep. Neurosci. Biobehav. Rev. 2016, 71, 671-679.
Roth, B. L.; Berry, S. A.; Kroeze, W. K.; Willins, D. L.; Kristiansen, K. Serotonin 5-ht2a receptors: Molecular biology and mechanisms of regulation. Crit. Rev. Neurobiol. 1998, 12, 319-338.
Singh, K.; Sona, C.; Ojha, V.; Singh, M.; Mishra, A.; Kumar, A.; Siddiqi, M. I.; Tripathi, R. P.; Yadav, P. N. Identification of dual role of piperazine-linked phenylcyclopropyl methanone as positive allosteric modulator of 5-ht(2c) and negative allosteric modulator of 5-ht(2b) receptors. Eur. J. Med. Chem. 2019, 164, 499-516.
Soogrim, V.; Ruberto, V. L.; Murrough, J.; Jha, M. K. Spotlight on pimavanserin tartrate and its therapeutic potential in the treatment of major depressive disorder: The evidence to date. Drug Des. Devel. Ther. 2021, 15, 151-157.
Soria-Gómez, E.; Marquez-Diosdado, M. I.; Montes-Rodriguez, C. J.; Estrada-González, V.; Prospéro-Garcia, O. Oleamide administered into the nucleus accumbens shell regulates feeding behaviour via cb1 and 5-ht2c receptors. Int. J. Neuropsychopharmacol. 2010, 13, 1247-1254.
Tecott, L. H.; Sun, L. M.; Akana, S. F.; Strack, A. M.; Lowenstein, D. H.; Dallman, M. F.; Julius, D. Eating disorder and epilepsy in mice lacking 5-ht2c serotonin receptors. Nature 1995, 374, 542-546.
Thomas, E. A.; Carson, M. J.; Neal, M. J.; Sutcliffe, G. Unique allosteric regulation of 5-hydroxytryptamine receptor-mediated signal transduction by oleamide. Proc. Natl. Acad. Sci. U.S.A 1997, 94, 14115-14119.
Thomas, E. A.; Carson, M. J.; Neal, M. J.; Sutcliffe, J. G. Unique allosteric regulation of 5-hydroxytryptamine receptor-mediated signal transduction by oleamide. Proc. Natl. Acad. Sci. U.S.A 1997, 94, 14115-14119.
Wager, T. T.; Hou, X.; Verhoest, P. R.; Villalobos, A. Central nervous system multiparameter optimization desirability: Application in drug discovery. ACS Chem. Neurosci. 2016, 7, 767-775.
Wager, T. T.; Hou, X.; Verhoest, P. R.; Villalobos, A. Moving beyond rules: The development of a central nervous system multiparameter optimization (cns mpo) approach to enable alignment of druglike properties. ACS Chem. Neurosci. 2010, 1, 435-449.
Wild, C. T.; Miszkiel, J. M.; Wold, E. A.; Soto, C. A.; Ding, C.; Hartley, R. M.; White, M. A.; Anastasio, N. C.; Cunningham, K. A.; Zhou, J. Design, synthesis, and characterization of 4-undecylpiperidine-2-carboxamides as positive allosteric modulators of the serotonin (5-ht) 5-ht(2c) receptor. J. Med. Chem. 2019, 62, 288-305.
Wild, C.; Cunningham, K. A.; Zhou, J. Allosteric modulation of g protein-coupled receptors: An emerging approach of drug discovery. Austin. J. Pharmacol. Ther. 2014, 2.
Wold, E. A.; Chen, J.; Cunningham, K. A.; Zhou, J. Allosteric modulation of class a gpcrs: Targets, agents, and emerging concepts. J. Med. Chem. 2019, 62, 88-127.
Wold, E. A.; Garcia, E. J.; Wild, C. T.; Miszkiel, J. M.; Soto, C. A.; Chen, J.; Pazdrak, K.; Fox, R. G.; Anastasio, N. C.; Cunningham, K. A.; Zhou, J. Discovery of 4-phenylpiperidine-2-carboxamide analogs as serotonin 5-ht2c receptor-positive allosteric modulators with enhanced drug-like properties. J. Med. Chem. 2020, 63, 7529-7544.
Wold, E. A.; Wild, C. T.; Cunningham, K. A.; Zhou, J. Targeting the 5-ht2c receptor in biological context and the current state of 5-ht2c receptor ligand development. Curr. Top. Med. Chem. 2019, 19, 1381-1398.
Wold, E. A.; Zhou, J. Gpcr allosteric modulators: Mechanistic advantages and therapeutic applications. Curr. Top. Med. Chem. 2018, 18, 2002-2006.
Zhang, G.; Stackman, R. W., Jr. The role of serotonin 5-ht2a receptors in memory and cognition. Front. Pharmacol. 2015, 6, 225.
Zhou, J.; Cunningham, K. A. Positive-allosteric modulation of the 5-ht(2c) receptor: Implications for neuropsychopharmacology and neurotherapeutics. Neuropsychopharmacology 2019, 44, 230-231.

We claim:

1. A compound according to Formula I: 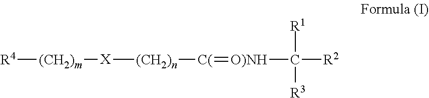
or a pharmaceutically acceptable salt thereof,
wherein:
R¹, R² and R³ are independently chosen from H, substituted or unsubstituted aryl, heteroaryl, arylalkyl, heteroaryl alkyl, carbonyl, C1 to C6 alkyl, C1 to C6 heteroalkyl, C1 to C6 alkoxy, C1 to C6 hydroxyalkyl, C1 to C6 ester, heterocyclyl;
R⁴ is chosen from H, substituted or unsubstituted aryl, substituted or unsubstituted heteroaryl, arylalkyl and heteroaryl alkyl;
X is selected from the group consisting of:
—CH₂CH₂—, —CH═CH—, 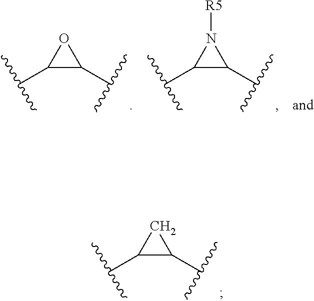
R⁵ is chosen from H, C1-C6 alkyl, and arylalkyl;
m is 0-20; and
n is 1-20.

2. The compound according to claim 1, or a pharmaceutically acceptable salt thereof, wherein X is —CH═CH—.

3. The compound according to claim 2, or a pharmaceutically acceptable salt thereof, wherein the —CH═CH— group is the cis-isomer.

4. The compound according to claim 3, or a pharmaceutically acceptable salt thereof, wherein m is 8, n is 7, and R⁴ is H.

5. The compound according to claim 1, or a pharmaceutically acceptable salt thereof, wherein X is

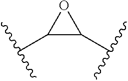 [SMILES: *C1OC1*]

6. The compound according to claim 1, or a pharmaceutically acceptable salt thereof, wherein X is

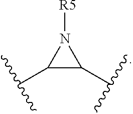

7. The compound according to claim 1, or a pharmaceutically acceptable salt thereof, wherein X is

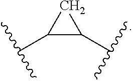 [SMILES: *C1CC1*]

8. The compound according to claim 1, or a pharmaceutically acceptable salt thereof, wherein X is —CH₂CH₂—.

9. The compound according to claim 1, or a pharmaceutically acceptable salt thereof, wherein R⁴ is chosen from H, substituted or unsubstituted aryl, substituted or unsubstituted heteroaryl, arylalkyl and heteroaryl alkyl.

10. The compound according to claim 1, chosen from any of: 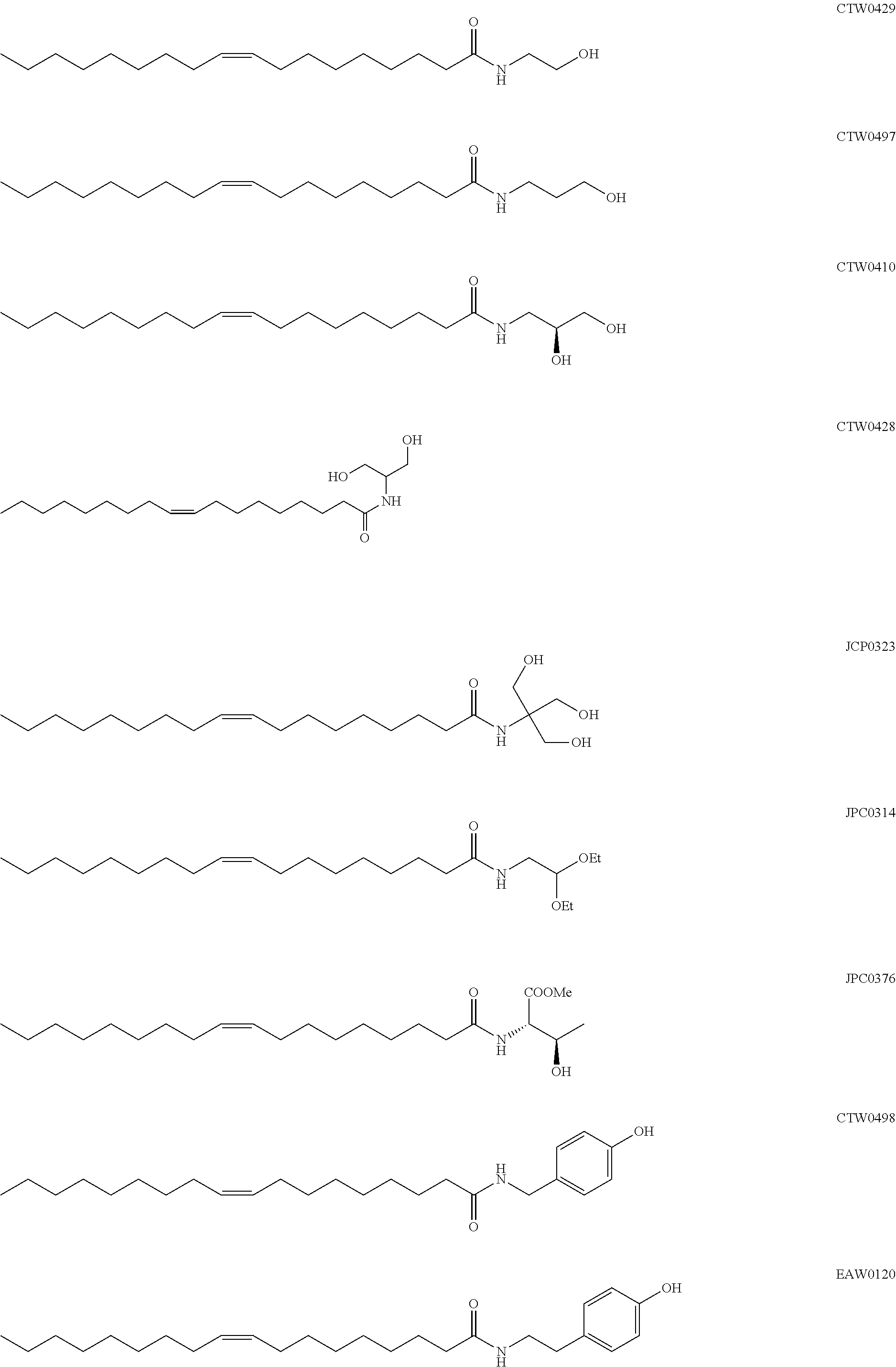 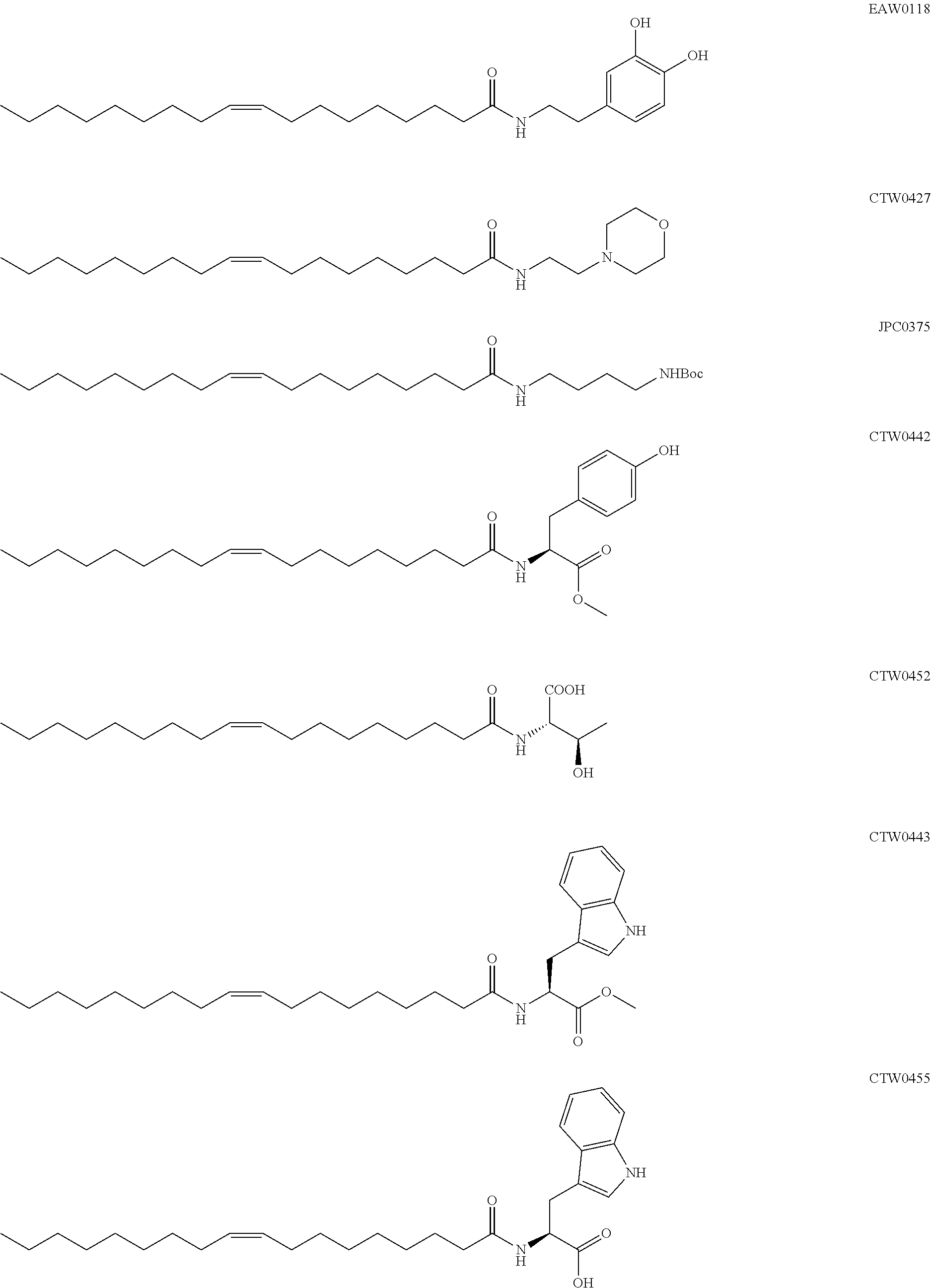 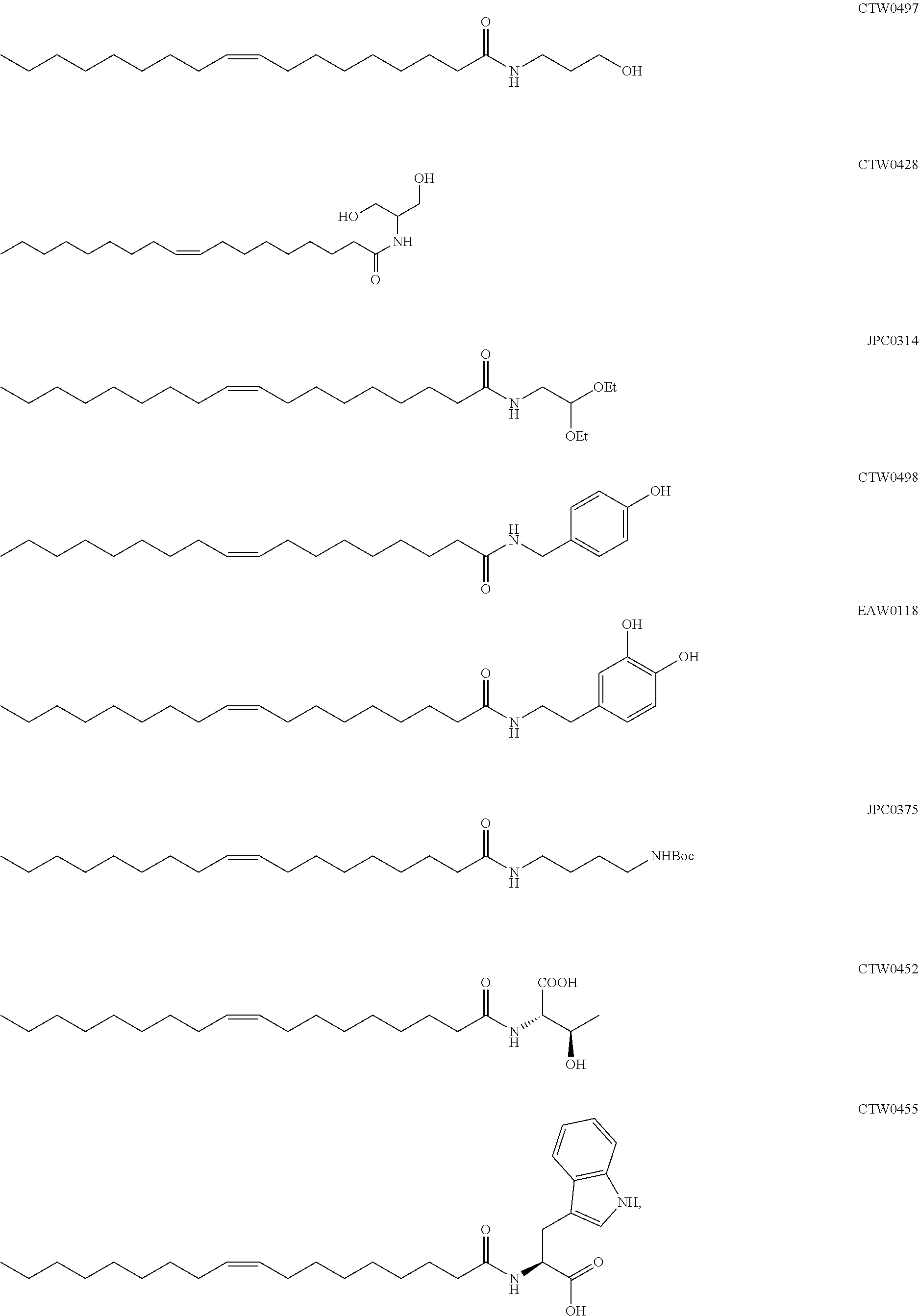
or a pharmaceutically acceptable salt thereof.

11. The compound according to claim 1, chosen from any of: 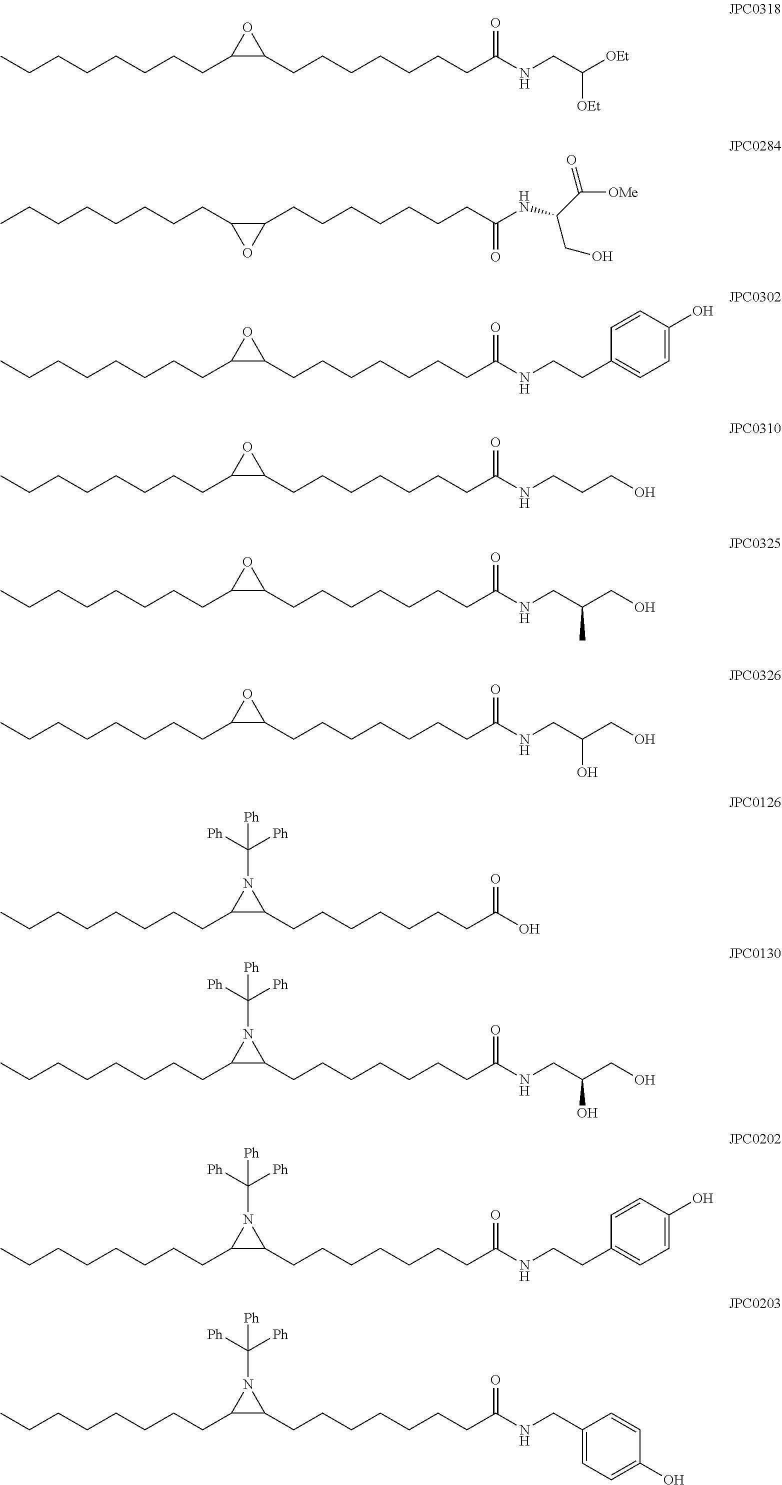 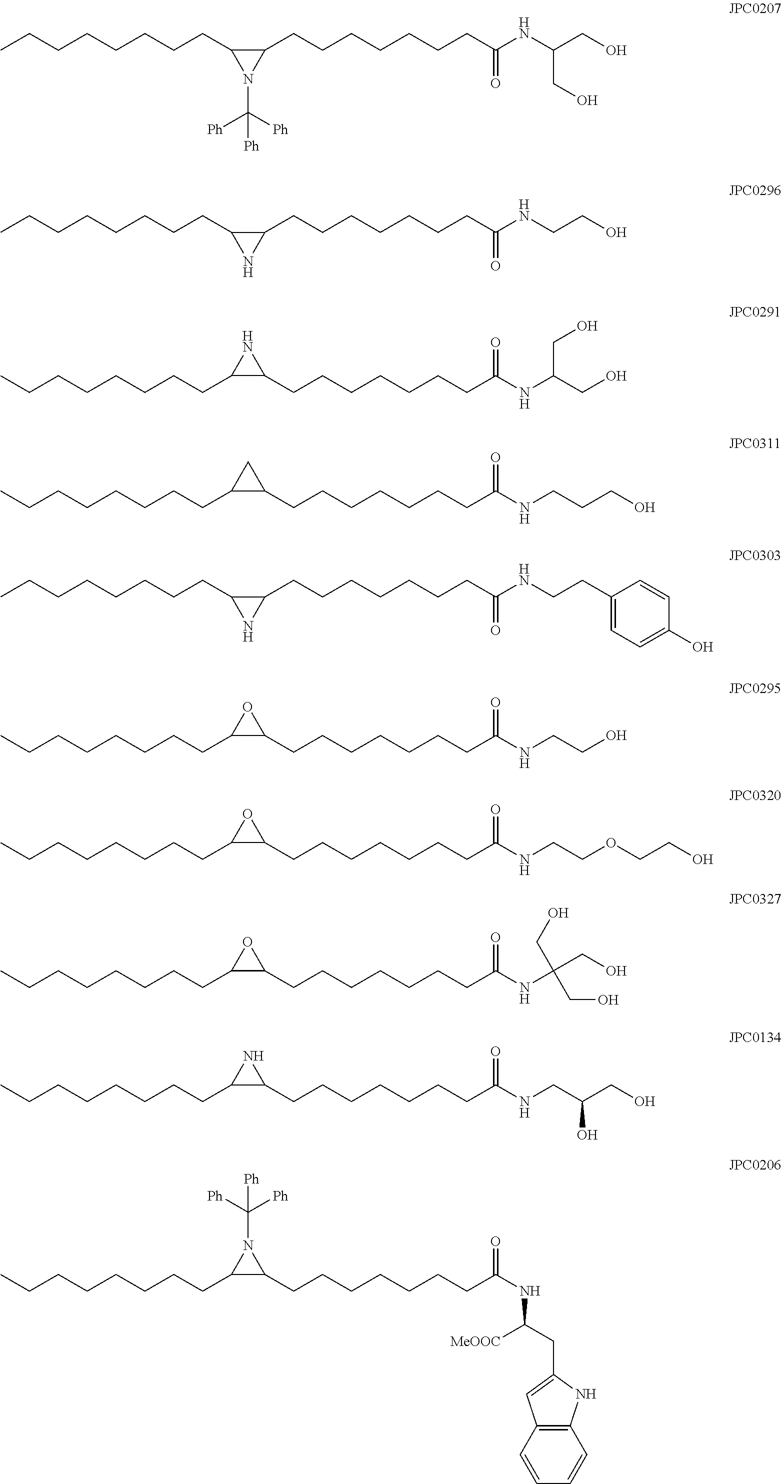 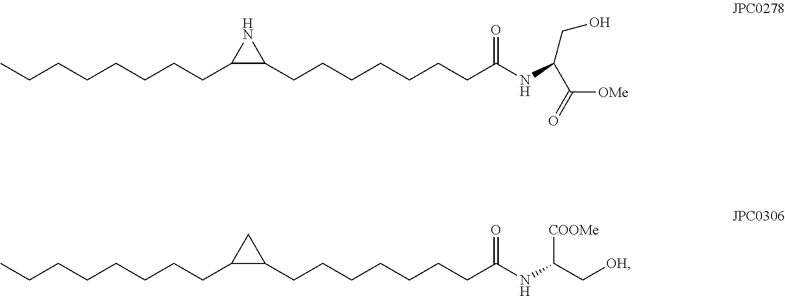 [SMILES: CCCCCCCCC1NC1CCCCCCCC(=O)N[C@@H](CO)C(=O)OC ; CCCCCCCCC1CC1CCCCCCCC(=O)N[C@@H](CO)C(=O)OC]
or a pharmaceutically acceptable salt thereof.

12. The compound according to claim 1, chosen from any of: 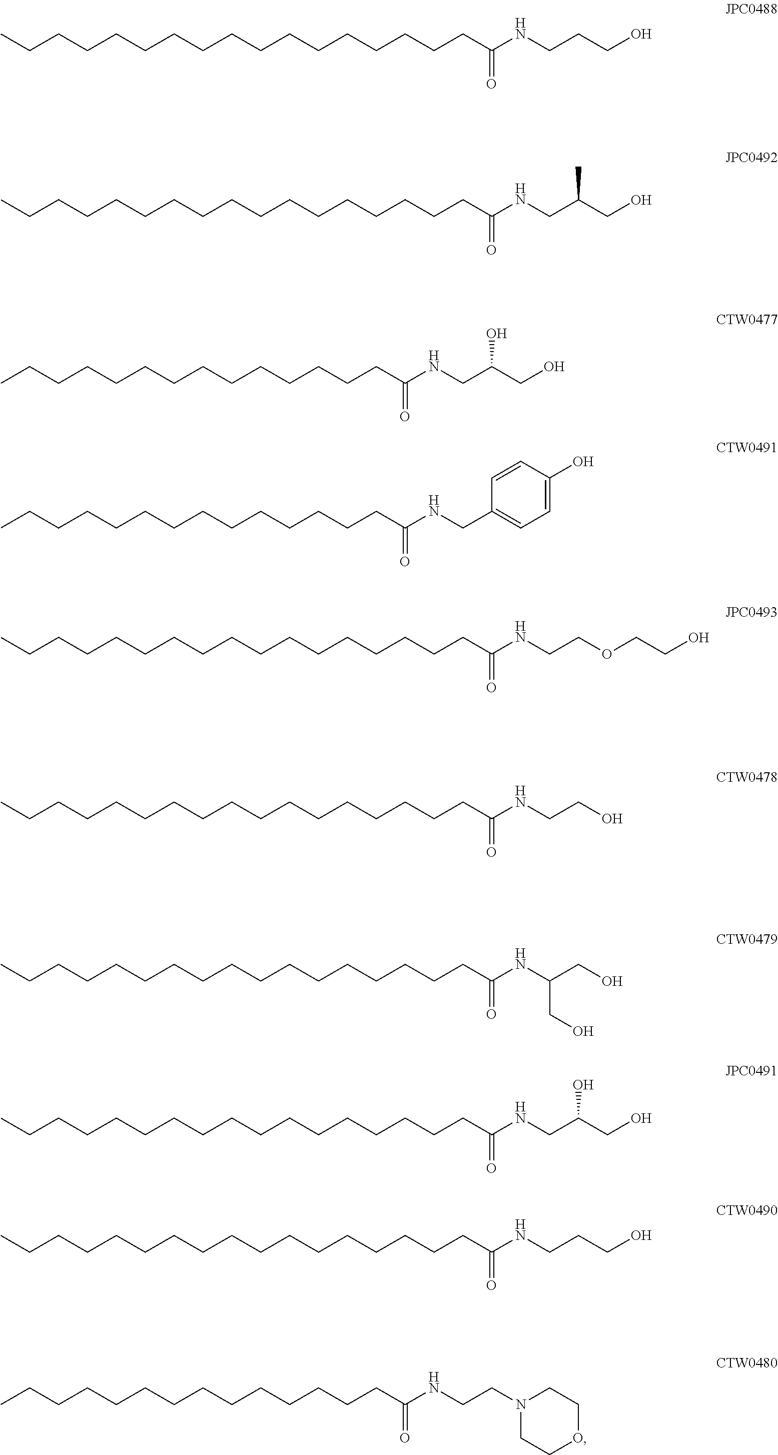
or a pharmaceutically acceptable salt thereof.

13. The compound according to claim 1, chosen from any of: 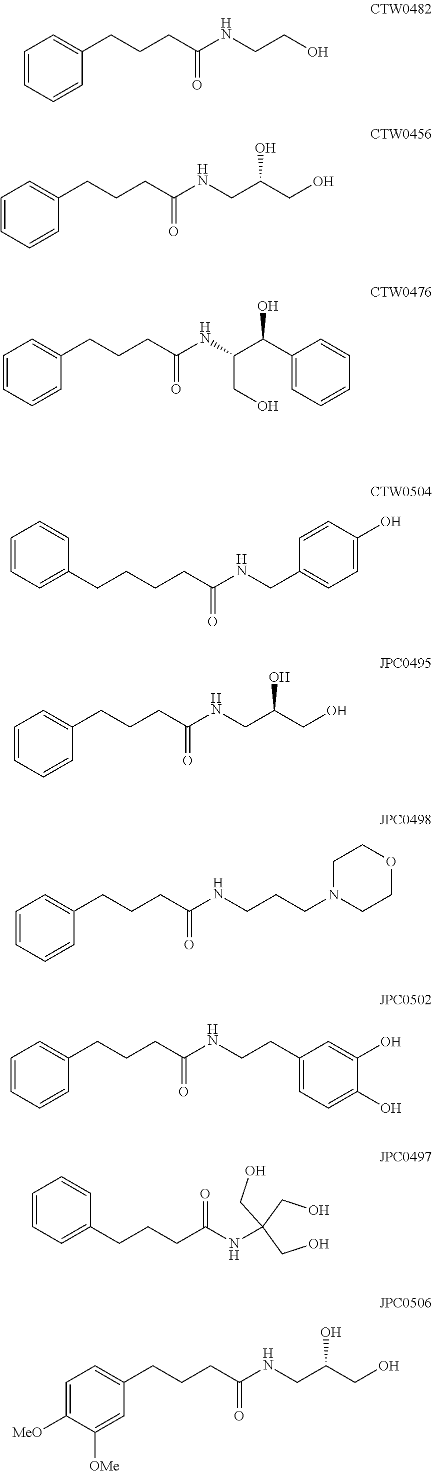 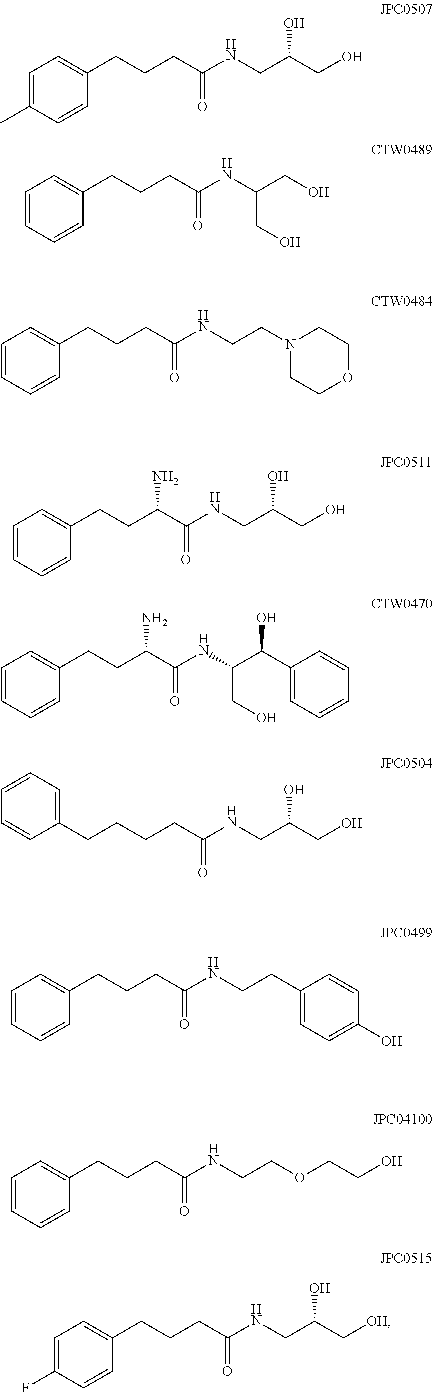
or a pharmaceutically acceptable salt thereof.

14. A compound according to Formula (II): 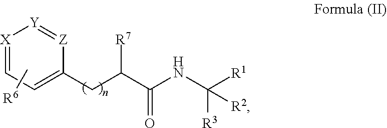
or a pharmaceutically acceptable salt thereof,
wherein R¹, R², R³ are independently chosen from H, substituted or unsubstituted aryl, heteroaryl, arylalkyl, heteroaryl alkyl, carbonyl, C1 to C6 alkyl, C1 to C6 heteroalkyl, C1 to C6 alkoxy, C1 to C6 hydroxyalkyl, and C1 to C6 ester;
R⁶is chosen from H, OH, NO₂, amino, CF₃, halogen, alkyl, and alkoxy;
R⁷ is H or NR⁸R⁹;
R⁸, R⁹ are independently chosen from H, alkyl, aryl, arylalkyl, cycloalkyl, alkoxy, and heteroalkyl, or
R⁸, R⁹ taken together with other atoms to form a 5- or 6-membered ring;
X, Y, Z are independently chosen from CH and N; and
n is 1 to 7.

15. The compound according to claim 14, or a pharmaceutically acceptable salt thereof, wherein the structure of the compound is selected from Formula II-(R) and Formula II-(S): 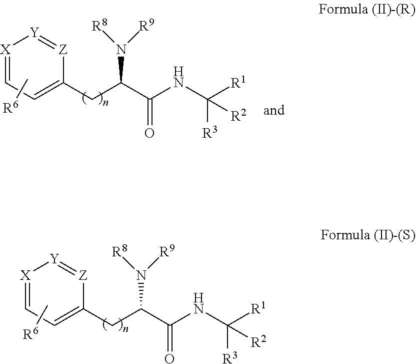
wherein the Formula II-(R) and Formula II-(S) designations refer to the respective R and S configurations at the chiral center carbon bonded to —NR⁸R⁹.

16. A method of treating a disease or condition, said method comprising administering to a patient a therapeutically effective amount of a compound according to Formula I 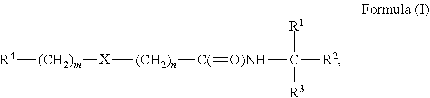
or a pharmaceutically acceptable salt thereof:
wherein:
R¹, R² and R³ are independently chosen from H, substituted or unsubstituted aryl, heteroaryl, arylalkyl, heteroaryl alkyl, carbonyl, C1 to C6 alkyl, C1 to C6 heteroalkyl, C1 to C6 alkoxy, C1 to C6 hydroxyalkyl, C1 to C6 ester, heterocyclyl;
R⁴ is selected from H, substituted or unsubstituted aryl, substituted or unsubstituted heteroaryl, arylalkyl and heteroaryl alkyl;
X is selected from the group consisting of:
—CH₂CH₂—, —CH═CH—, 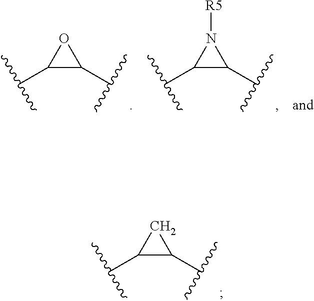
R⁵ is selected from H, C1-C6 alkyl;
m is 0-20; and
n is 1-20.

17. The method according to claim 16, wherein treating said disease or condition is associated with the modulation of 5-hydroxytryptamine 2A receptor and/or 5-hydroxytryptamine 2C receptor.

18. The method according to claim 16, wherein said disease or condition may be treated by 15 modulating 5-hydroxytryptamine 2A receptor and/or 5-hydroxytryptamine 2C receptor.

19. The method according to claim 16, wherein said compound according to Formula I modulates 5-hydroxytryptamine 2A receptor and/or 5-hydroxytryptamine 2C receptor.

20. The method according to claim 16, wherein said disease or condition responsive is a substance use disorder, obesity, a mood disorder or a seizure disorder.

21. A method of treating a disease or condition, said method comprising administering to a patient a therapeutically effective amount of a compound chosen from any of Formulas Ia, II, II-(R), and II-(S), or a combination thereof, or a pharmaceutically acceptable salt thereof.

22. The method according to claim 21, wherein treating said disease or condition is associated with modulation of 5-hydroxytryptamine 2A receptor and/or 5-hydroxytryptamine 2C receptor.

23. The method according to claim 21, wherein said disease or condition is treated by modulating 5-hydroxytryptamine 2A receptor and/or 5-hydroxytryptamine 2C receptor.

24. The method according to claim 21, wherein said compound(s), or a pharmaceutically acceptable salt thereof, modulates 5-hydroxytryptamine 2A receptor and/or 5-hydroxytryptamine 2C receptor.

25. The method according to claim 21, wherein said disease or condition responsive is a substance use disorder, obesity, a mood disorder or a seizure disorder.
